SEQUENCE LISTING (13 sequences)

SEQ ID NO: 1 - protein, 775 aa, Pyrococcus horikoshii
MILDADYITEDGKPIIRIFKKENGEFKVEYDRNFRPYIYALLRDDSAIDEIKKITAQRHGKVVRIVETEKIQRKFLGRPIEVWKLYLEHPQDVPAIRDKIREHPAVVDIFEYDIPFAKRYLIDKGLTPMEGNEKLTFLAVDIETLYHEGEEFGKGPVIMISYADEEGAKVITWKKIDLPYVEVVSSEREMIKRLIRVIKEKDPDVIITYNGDNFDFPYLLKRAEKLGIKLLLGRDNSEPKMQKMGDSLAVEIKGRIHFDLFPVIRRTINLPTYTLEAVYEAIFGKPKEKVYADEIAKAWETGEGLERVAKYSMEDAKVTYELGREFFPMEAQLARLVGQPVWDVSRSSTGNLVEWFLLRKAYERNELAPNKPDEKEYERRLRESYEGGYVKEPEKGLWEGIVSLDFRSLYPSIIITHNVSPDTLNREGCEEYDVAPKVGHRFCKDFPGFIPSLLGQLLEERQKIKKRMKESKDPVEKKLLDYRQRAIKILANSYYGYYGYAKARWYCKECAESVTAWGRQYIDLVRRELEARGFKVLYIDTDGLYATIPGVKDWEEVKRRALEFVDYINSKLPGVLELEYEGFYARGFFVTKKKYALIDEEGKIVTRGLEIVRRDWSEIAKETQARVLEAILKHGNVEEAVKIVKDVTEKLTNYEVPPEKLVIYEQITRPINEYKAIGPHVAVAKRLMARGIKVKPGMVIGYIVLRGDGPISKRAISIEEFDPRKHKYDAEYYIENQVLPAVERILKAFGYKREDLRWQKTKQVGLGAWIKVKKS

SEQ ID NO: 2 - DNA, 2328 bp, Pyrococcus horikoshii
  source 1..2328: mol_type = other DNA
atgattctggacgctgattatattactgaagatggtaaaccgattattcgtatttttaaaaaagaaaatggcgagttcaaagttgaatatgaccgtaactttcgtccgtacatctacgcgctgttgcgcgacgatagcgcgatcgatgagattaagaaaattaccgcgcagcgtcatggtaaagttgttcgcatcgttgaaaccgagaaaattcaacgtaaattcctgggccgcccaattgaagtgtggaagctgtacctggagcatccgcaagatgtcccggcgatccgtgacaagattcgcgagcacccggccgtcgtcgacattttcgaatacgatattccgttcgcaaagcgttacctgatcgataagggtctgaccccgatggagggtaatgaaaagctgacgttcctggctgtcgatattgaaacgttgtaccacgagggtgaagagtttggtaagggcccggtcattatgatcagctacgcggatgaagagggcgccaaagttatcacgtggaaaaaaattgatctgccgtacgttgaagttgtgtccagcgagcgcgagatgattaaacgcttgattcgtgtgattaaagaaaaagatccagacgtgatcattacctataatggtgacaactttgactttccgtacttgctgaaacgtgctgagaaactgggtatcaagctgttgctgggtcgcgataatagcgagccgaagatgcaaaaaatgggcgatagcctggcagtcgagatcaagggtcgcatccactttgatctctttccggtgattcgtcgcacgatcaatctgccgacctatacgctggaagctgtctacgaggcaatctttggtaagccgaaagaaaaagtctatgcggacgaaattgcgaaagcgtgggaaaccggcgagggcctggagcgtgtggcaaagtactctatggaagatgccaaagtgacctatgaactgggtcgtgagttcttcccaatggaagcccagttggcgcgcttggtgggccaaccggtttgggacgtttcccgtagcagcaccggtaacctggttgagtggtttctgttgcgtaaagcgtatgagcgtaatgaactggcaccgaacaagcctgacgagaaagaatatgaacgtcgcctgcgtgaatcttacgagggtggttacgtcaaagaaccggaaaagggtctgtgggaaggcatcgtgagcctggatttccgtagcctgtaccctagcatcatcatcacgcacaatgttagcccggacaccctgaaccgcgagggctgcgaagagtacgacgttgcgccgaaagtcggccatcgtttttgtaaagacttccctggtttcatcccaagcctgctgggtcagctgctggaagagagacagaaaattaaaaaacgcatgaaagaatcgaaagatccggttgagaaaaagctgctggattaccgccagcgtgccatcaagattctggctaactcatattatggctactacggttatgctaaagcgcgttggtactgtaaagagtgcgcggagtccgtcaccgcgtggggtcgccagtatatcgatctggtgcgtcgcgagctggaagcgcgtggttttaaggtcctgtacatcgatactgacggtctgtatgcaaccatccctggtgtcaaagactgggaagaggttaagcgtcgtgcactggaatttgtggactatatcaattctaagttgccgggtgtgctggagctggagtacgaaggcttctatgcacgcggctttttcgttacgaaaaagaaatacgcactgatcgacgaagagggcaagattgtgactcgtggtctggaaatcgttcgtcgcgactggagcgagattgcaaaagaaacccaagctcgcgttctggaagcaatcctgaaacatggtaacgtcgaagaagccgtcaagatcgtgaaagatgtcaccgaaaagttgaccaactacgaagttccaccggaaaaactggtgatttatgagcaaatcacgcgtccgatcaatgaatataaggccattggcccgcacgtcgcggtggccaagcgcctgatggcgcgtggtatcaaagtgaaaccgggtatggttattggttacatcgtgctgcgtggcgacggcccgattagcaaacgtgcgatcagcattgaagaatttgacccgcgtaagcacaaatatgacgcggaatactatatcgagaatcaagtgctgccggccgtggaacgcattctgaaagctttcggctacaagcgtgaagatttgcgctggcagaaaaccaaacaggttggtcttggtgcgtggatcaaggtcaaaaagtcctaa

SEQ ID NO: 3 - protein, 771 aa, Pyrococcus sp.
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVDIETLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPVIRRTINLPTYTLEAVYEAIFGKSKEKVYAHEIAEAWETGKGLERVAKYSMEDAKVTFELGKEFFPMEAQLARLVGQPVWDVSRSSTGNLVEWFLLRKAYERNELAPNKPDEREYERRLRESYEGGYVKEPEKGLWEGIVSLDFRSLYPSIIITHNVSPDTLNRENCKEYDVAPQVGHRFCKDFPGFIPSLLGNLLEERQKIKKRMKESKDPVEKKLLDYRQRAIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLKYQKTKQVGLGAWLKF

SEQ ID NO: 4 - protein, 775 aa, Pyrococcus woesei
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 5 - protein, 775 aa, Pyrococcus furiosus; identical to SEQ ID NO: 4

SEQ ID NO: 6 - protein, 775 aa, Pyrococcus glycovorans
MILDADYITEDGKPIIRIFKKENGEFKVEYDRNFRPYIYALLKDDSQIDEVKKITAERHGKIVRIVDVEKVKKKFLGRPIEVWKLYFEHPQDVPAIRDKIREHPAVVDIFEYDIPFAKRYLIDKGLIPMEGDEELKLLAFDIETLYHEGEEFAKGPIIMISYADEEGAKVITWKKVDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCMEYDVAPEVKHKFCKDFPGFIPSLLKRLLDERQEIKRRMKASKDPIEKKMLDYRQRAIKILANSYYGYYGYAKARWYCKECAESVTAWGREYIEFVRKELEEKFGFKVLYIDTDGLYATIPGAKPEEIKRKALEFVEYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDPRKHKYDAEYYIENQVLPAVLRILEAFGYRKEDLRWQKTKQTGLTAWLNVKKK

SEQ ID NO: 7 - protein, 775 aa, Pyrococcus sp.
MILDADYITEDGKPIIRLFKKENGRFKVEYDRNFRPYIYALLKDDSAIDDVRKITSERHGKVVRVIDVEKVKKKFLGRPIEVWKLYFEHPQDVPAMRDKIREHPAVIDIFEYDIPFAKRYLIDKGLIPMEGNEELTFLAVDIETLYHEGEEFGKGPIIMISYADEEGAKVITWKKIDLPYVEVVANEREMIKRLIKVIREKDPDVIITYNGDNFDFPYLLKRAEKLGMKLPLGRDNSEPKMQRLGDSLAVEIKGRIHFDLFPVIRRTINLPTYTLEAVYEAIFGKQKEKVYPHEIAEAWETGKGLERVAKYSMEDAKVTYELGKEFFPMEAQLARLVGQPLWDVSRSSTGNLVEWYLLRKAYERNELAPNKPDEREYERRLRESYEGGYVKEPERGLWEGIVSLDFRSLYPSIIITHNVSPDTLNKEGCGEYDEAPEVGHRFCKDFPGFIPSLLGSLLEERQKIKKRMKESKDPVERKLLDYRQRAIKILANSFYGYYGYAKARWYCKECAESVTAWGRQYIELVRRELEERGFKVLYIDTDGLYATIPGEKNWEEIKRRALEFVNYINSKLPGILELEYEGFYTRGFFVTKKKYALIDEEGKIVTRGLEIVRRDWSEIAKETQAKVLEAILKHGNVEEAVKIVKEVTEKLSNYEIPVEKLVIYEQITRPLNEYKAIGPHVAVAKRLAAKGIKIKPGMVIGYVVLRGDGPISKRAIAIEEFDGKKHKYDAEYYIENQVLPAVERILKAFGYKREDLRWQKTKQVGLGAWLKVKKS

SEQ ID NO: 8 - protein, 759 aa, Pyrococcus sp.
MILDADYITENGKPIIRLFKKENGKFKVEYDRNFRPYIYALLKDDSAIDDVRKITSERHGKVVRVIDVEKVSKKFLGRPIEVWKLYFEHPQDVPAIRDKIREHPAVIDIFEYDIPFAKRYLIDKGLIPMEGNEELSFLAVDIETLYHEGEEFGKGPIIMISYADEEGAKVITWKKIDLPYVEVVANEREMIKRLVRIIREKDPDIIITYNGDNFDFPYLLKRAEKLGIKLPLGRDNSEPKMQRLGESLAVEIKGRIHFDLFPVIRRTINLPTYTLRTVYEAIFGKPKEKVYPHEIAEAWETGKGLERVAKYSMEDAKVTYELGKEFFPMEAQLARLVGQPVWDVSRSSTGNLVEWFLLRKAYERNELAPNKPDEKEYEKRLRESYEGGYVKEPEKGLWEGIVSLDFRSLYPSIIITHNVSPDTLNREGCGKYDEAPEVGHRFCKDFPGFIPSLLGDLLEERQKIKKRMKESKDPIEKKLLDYRQRAIKILANSFYGYYGYAKARWYCKECAESVTAWGRQYIELVRRELEERGFKVLYIDTDGLYATIPGEKNWEEIKRKALEFVNYINSKLPGILELEYEGFYTRGFFVTKKKYALIDEEGKIITRGLEIVRRDWSEIAKETQAKVLEAILKHGNVEEAVKIVKEVTEKLSNYEIPVEKLVIYEQITRPLNEYKAIGPHVAVAKRLAAKGIKIKPGMVIGYVLLRGDGPISKRAIAIEEFDGKKHKYDAEYYIENQVLPAVERILKAFGYKKEDLRWQ

SEQ ID NO: 9 - protein, 775 aa, Pyrococcus sp.
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKK

SEQ ID NO: 10 - protein, 776 aa, Pyrococcus yayanosii
MILDADYITENGKPVVRIFKKENGEFKVEYDRSFRPYIYALLRDDSAIEDIKKITAERHGKVVRVVEAEKVRKKFLGRPIEVWKLYFEHPQDVPAIREKIREHPAVIDIFEYDIPFAKRYLIDKGLIPMEGNEELKLLAFDIETLYHEGDEFGSGPIIMISYADEKGAKVITWKGVDLPYVEVVSSEREMIKRFLRVIREKDPDVIITYNGDNFDFPYLLKRAEKLGMKLPIGRDGSEPKMQRMGDGFAVEVKGRIHFDIYPVIRRTINLPTYTLEAVYEAVFGRPKEKVYPNEIARAWENCKGLERVAKYSMEDAKVTYELGREFFPMEAQLARLVGQPVWDVSRSSTGNLVEWFLLRKAYERNELAPNRPDEREYERRLRESYEGGYVKEPEKGLWEGIIYLDFRSLYPSIIITHNISPDTLNKEGCNSYDVAPKVGHRFCKDFPGFIPSLLGQLLDERQKIKRKMKATIDPIERKLLDYRQRAIKILANSYYGYYGYAKARWYCKECAESVTAWGREYIELVSRELEKRGFKVLYIDTDGLYATIPGSREWDKIKERALEFVKYINARLPGLLELEYEGFYKRGFFVTKKKYALIDEEGKIITRGLEIVRRDWSEIAKETQARVLEAILKEGNLEKAVKIVKEVTEKLSKYEVPPEKLVIYEQITRDLKDYKAVGPHVAVAKRLAARGIKVRPGMVIGYLVLRGDGPISRRAIPAEEFDPSRHKYDAEYYIENQVLPAVLRILEAFGYRKEDLRYQKTRQAGLDAWLKRKASL

SEQ ID NO: 11 - protein, 774 aa, Pyrococcus sp.
MILDADYITEDGKPVIRLFKKENGEFKIEYDRTFKPYIYALLKDDSAIDEVRKVTAERHGKIVRIIDVEKVKKKYLGRPIEVWKLYFEHPQDVPAIREKIREHPAVVEIFEYDIPFAKRYLIDKGIVPMDGDEELKLLAFDIETLYHEGEEFGKGPILMISYADEEGAKVITWKRINLPYVEVVSSEREMIKRFLKVIREKDPDVIITYNGDSFDFPYLVKRAEKLGIKLPLGRDGSPPKMQRLGDMNAVEIKGRIHFDLYHVVRRTINLPTYTLEAVYEAIFGKPKEKVYAHEIAEAWETGKGLERVAKYSMEDAQVTYELGKEFFPMEVQLTRLVGQPLWDVSRSSTGNLVEWYLLRKAYERNELAPNKPDEREYERRLRESYAGGYVKEPERGLWENIVYLDFRSLYPSIIITHNVSPDTLNREGCRKYDIAPEVGHKFCKDVEGFIPSLLGHLLEERQKIKRKMKATINPVEKKLLDYRQKAIKILANSYYGYYGYAKARWYCKECAESVTAWGREYIELVRKELEGKFGFKVLYIDTDGLYATIPRGDPAEIKKKALEFVRYINEKLPGLLELEYEGFYRRGFFVTKKKYALIDEEDKIITRGLEIVRRDWSEIAKETQAKVLEAILKEGNVEKAVKIVKEVTEKLMKYEVPPEKLVIYEQITRPLNEYKAIGPHVAVAKRLAAKGVKVRPGMVIGYIVLRGDGPISKRAILAEEYDPRKNKYDAEYYIENQVLPAVLRILEAFGYKKEDLKYQKSRQVGLGAWIKVKK

SEQ ID NO: 12 - protein, 775 aa, Pyrococcus sp.; identical to SEQ ID NO: 9

SEQ ID NO: 13 - protein, 775 aa, synthetic construct
MILDADYITEDGKPIIRIFKKENGEFKVEYDRNFRPYIYALLRDDSAIDEIKKITAQRHGKVVRIVETEKIQRKFLGRPIEVWKLYLEHPQDQPAIRDKIREHPAVVDIFEYDIPFAKRYLIDKGLTPAEGNEKLTFLAVAIAALYHEGEEFGKGPVIMISYADEEGAKVITWKKIDLPYVEVVSSEREMIKRLIRVIKEKDPDVIITYNGDNFDFPYLLKRAEKLGIKLLLGRDNSEPKMQKMGDSLAVEIKGRIHFDLFPVIRRTINLPTYTLEAVYEAIFGKPKEKVYADEIAKAWETGEGLERVAKYSMEDAKVTYELGREFFPMEAQLARLVGQPVWDVSRSSTGNLVEWFLLRKAYERNELAPNKPDEKEYERRLRESYEGGYVKEPEKGLWEGIVSLDFRSAGPSIIITHNVSPDTLNREGSEEYDVAPKVGHRFSKDFPGFIPSLLGQLLEERQKIKKRMKESKDPVEKKLLDYRQRVIKILANSYYGYYGYAKARWYSKESAESVSAWGRQYIDLVRRELEARGFKVLYIDTDGLYATIPGVKDWEEVKRRALEFVDYINSKLPGVLELEYEGFYARGFFVTKKKYALIDEEGKIVTRGLEIVRRDWSEIAKETQARVLEAILKHGNVEELVKIVKDVTEKLTNYEVPPEKLVIYEQITRPINEYKAIGPHVAVAKRLMARGIKVKPGMVIGYIVLRGDGPISKRAISIEEFDPRKHKYDAEYYIENQVLPAVERILKAFGYKREDLRWQKTKQVGLGAWIKVKKS

(19) United States
(12) Patent Application Publication
(10) Pub. No.: US 2023/0203578 A1
(43) Pub. Date: Jun. 29, 2023

(54) STRAND DISPLACING SEQUENCING ENZYMES

(71) Applicant: Singular Genomics Systems, Inc., San Diego, CA (US)

(72) Inventors: Souad NAJI, La Jolla, CA (US); Eli N. GLEZER, Del Mar, CA (US); Bruna Krebs KUTCHE, San Diego, CA (US); Ali NIKOOMANZAR, San Diego, CA (US); Alexander SEVY, San Diego, CA (US); Caleb NEWMAN, San Diego (UA); Ryan SHULTZABERGER, San Diego, CA (US); Abrehet ABDU, San Diego, CA (US); Youngjin CHO, San Diego, CA (US); Janell J. KOSMATKA, Encinitas, CA (US)

(21) Appl. No.: 18/069,990

(22) Filed: Dec. 21, 2022

Related U.S. Application Data
(60) Provisional application No. 63/292,885, filed on Dec. 22, 2021.

(51) Int. Cl. C12Q 1/6874; C12N 9/12

(52) U.S. Cl. CPC C12Q 1/6874; C12N 9/1252; C12Y 207/07007; C12Q 1/6818

(57) ABSTRACT

Disclosed herein, inter alia, are methods, enzymes, and compositions useful for nucleic acid sequencing.

### CROSS-REFERENCES TO RELATED APPLICATIONS

This application claims the benefit of U.S. Provisional Application No. 63/292,885 filed on Dec. 22, 2021, which is incorporated herein by reference in its entirety and for all purposes.

### REFERENCE TO SEQUENCE LISTING SUBMITTED ELECTRONICALLY

The Sequence Listing titled 051385-563001US_SL_ST26.XML, was created on Dec. 15, 2022 in machine format IBM-PC, MS-Windows operating system, is 23,573 bytes in size, and is hereby incorporated by reference in its entirety for all purposes.

### BACKGROUND

Genetic analysis is taking on increasing importance in modern society as a diagnostic, prognostic, and as a forensic tool, and typically requires amplification of genomic fragments. A majority of nucleic acid amplification techniques (e.g., DNA amplification) used in university, medical, and clinical laboratory research is performed using the polymerase chain reaction (PCR), though in the past decade alternative amplification methods have emerged that eliminate thermal cycling (i.e., isothermal amplification). Typical isothermal amplification methods require the use of a DNA polymerase with a strong strand displacement activity to displace downstream DNA, thereby enabling continuous replication without thermal cycling. Efficient amplification typically requires elevated temperatures to enable the annealing of primers at specific locations on the dsDNA. However, few thermostable strand displacing enzymes exist. For example, SD DNA polymerase (a mutant Taq DNA polymerase) and the large fragment of Bst DNA polymerase possess favorable characteristics for isothermal amplification, but both are inactivated and elevated temperatures (e.g., greater than 70° C.). In addition to nucleic acid amplification, polymerase mediated nucleic acid sequencing methods (e.g., sequencing-by-synthesis) benefit by using a thermostable, strand-displacing polymerase. Thus, there is a need for thermostable, strand-displacing polymerases. Disclosed herein, inter alia, are solutions to these and other problems in the art.

### BRIEF SUMMARY

In an aspect is provided a polymerase including an amino acid sequence that is at least 80% identical to a continuous 500 amino acid sequence within SEQ ID NO: 1; including a first mutation at amino acid position 409 or an amino acid position corresponding to position 409; and at least one mutation at amino acid position 7 or an amino acid position corresponding to position 7; at amino acid position 579 or an amino acid position corresponding to position 579; at amino acid position 588 or an amino acid position corresponding to position 588; or at amino acid position 742 or an amino acid position corresponding to position 742.

In an aspect is provided a method of incorporating a modified nucleotide into a nucleic acid sequence including combining in a reaction vessel: (i) a nucleic acid template, (ii) a nucleotide solution, and (iii) a polymerase, wherein the polymerase is a polymerase as described herein. In embodiments, the modified nucleotide includes a label (e.g., a label linked to the nucleobase via an optionally cleavable linker). In embodiments, the modified nucleotide includes a reversible terminator moiety (e.g., a polymerase-compatible cleavable moiety bonded to the 3′ oxygen of a nucleotide).

In another aspect is provided a method of sequencing a nucleic acid sequence including: a. hybridizing a nucleic acid template with a primer to form a primer-template hybridization complex; b. contacting the primer-template hybridization complex with a DNA polymerase and modified nucleotides, wherein the DNA polymerase is the polymerase as described herein, wherein the modified nucleotide includes a detectable label; c. subjecting the primer-template hybridization complex to conditions which enable the polymerase to incorporate a modified nucleotide into the primer-template hybridization complex to form a modified primer-template hybridization complex; and d. detecting the detectable label; thereby sequencing a nucleic acid sequence.

### BRIEF DESCRIPTION OF THE DRAWINGS

FIG. 1 illustrates the hairpin-challenge strand-displacing assay, which utilizes streptavidin bead, depicted as a large circle (bead) and diamond (streptavidin), conjugated to a dual hairpin challenge template.

FIG. 2 depicts an alignment of two sequences described herein. SEQ ID NO:3 is aligned to SEQ ID NO:1 and amino acid positions 541-560 are depicted in FIG. 2. The alignment highlights a deletion in SEQ ID NO:3 relative to SEQ ID NO:1, such that any amino acid positions beyond amino acid position 554 are shifted −1 in SEQ ID NO:3 relative to SEQ ID NO:1 (i.e., amino acid E554 in SEQ ID NO:3 corresponds to E555 in SEQ ID NO:1).

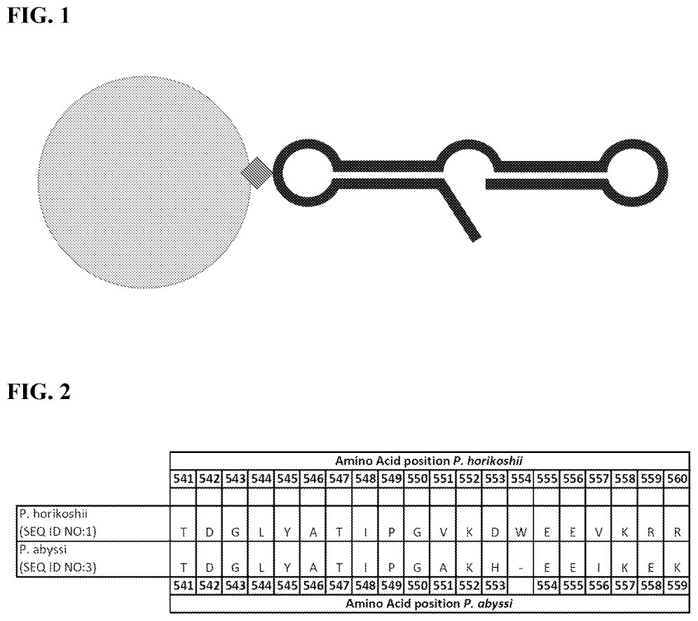

### DETAILED DESCRIPTION

The aspects and embodiments described herein relate to strand displacing polymerases and uses thereof.

### Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. Different polynucleotides may have different three-dimensional structures, and may perform various functions, known or unknown.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The term “base” and “nucleobase” as used herein refers to a purine or pyrimidine compound, or a derivative thereof, that may be a constituent of nucleic acid (i.e. DNA or RNA, or a derivative thereof). In embodiments, the base is a derivative of a naturally occurring DNA or RNA base (e.g., a base analogue). In embodiments, the base is a base-pairing base. In embodiments, the base pairs to a complementary base. In embodiments, the base is capable of forming at least one hydrogen bond with a complementary base (e.g., adenine hydrogen bonds with thymine, adenine hydrogen bonds with uracil, guanine pairs with cytosine). Non-limiting examples of a base includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), guanosine or a derivative thereof (e.g., 7-methylguanosine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the base is thymine, cytosine, uracil, adenine, guanine, hypoxanthine, xanthine, theobromine, caffeine, uric acid, or isoguanine. In embodiments, the base is

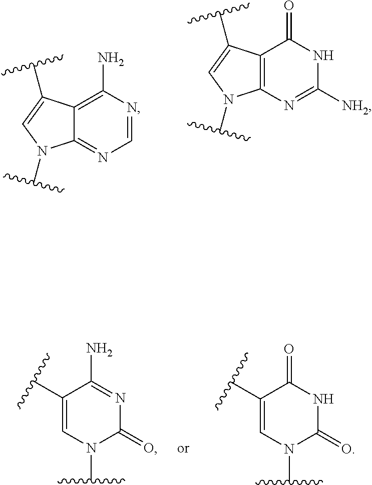 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “isolated”, when applied to a nucleic acid or protein, denotes that the nucleic acid or protein is essentially free of other cellular components with which it is associated in the natural state. It can be, for example, in a homogeneous state and may be in either a dry or an aqueous solution. Purity and homogeneity are typically determined using analytical chemistry techniques such as polyacrylamide gel electrophoresis or high performance liquid chromatography. A protein that is the predominant species present in a preparation is substantially purified.

The terms “analog” and “analogue” and “derivative” in reference to a chemical compound, refers to compounds having a structure similar to that of another one, but differing from it in respect of one or more different atoms, functional groups, or substructures that are replaced with one or more other atoms, functional groups, or substructures. In the context of a nucleotide useful in practicing the invention, a nucleotide analog refers to a compound that, like the nucleotide of which it is an analog, can be incorporated into a nucleic acid molecule (e.g., an extension product) by a suitable polymerase, for example, a DNA polymerase in the context of a dNTP analogue. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “complement,” as used herein, refers to a nucleotide (e.g., RNA or DNA) or a sequence of nucleotides capable of base pairing with a complementary nucleotide or sequence of nucleotides. As described herein and commonly known in the art, the complementary (matching) nucleoside of adenosine is thymidine and the complementary (matching) nucleoside of guanosine is cytidine. Thus, a complement may include a sequence of nucleotides that base pair with corresponding complementary nucleotides of a second nucleic acid sequence. The nucleotides of a complement may match, partially or completely, the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence, only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein, the complementarity of sequences may be partial, in which only some of the nucleic acids match according to base pairing, or complete, where all the nucleic acids match according to base pairing. Thus, two sequences that are complementary to each other may have a specified percentage of nucleotides that are complementary (i.e., about 60% identity, preferably 65%, 70%, 75%, 80%, 85%, 90%, 91%, 92%, 93%, 94%, 95%, 96%, 97%, 98%, 99%, or higher complementarity over a specified region).

“DNA” refers to deoxyribonucleic acid, a polymer of deoxyribonucleotides (e.g., dATP, dCTP, dGTP, dTTP, dUTP, etc.) linked by phosphodiester bonds. DNA can be single-stranded (ssDNA) or double-stranded (dsDNA), and can include both single and double-stranded (or “duplex”) regions. “RNA” refers to ribonucleic acid, a polymer of ribonucleotides linked by phosphodiester bonds. RNA can be single-stranded (ssRNA) or double-stranded (dsRNA), and can include both single and double-stranded (or “duplex”) regions. Single-stranded DNA (or regions thereof) and ssRNA can, if sufficiently complementary, hybridize to form double-stranded DNA/RNA complexes (or regions).

The term “DNA primer” refers to any DNA molecule that may hybridize to a DNA template and be bound by a DNA polymerase and extended in a template-directed process for nucleic acid synthesis. The term “DNA template” refers to any DNA molecule that may be bound by a DNA polymerase and utilized as a template for nucleic acid synthesis.

The term “dATP analogue” refers to an analogue of deoxyadenosine triphosphate (dATP) that is a substrate for a DNA polymerase. The term “dCTP analogue” refers to an analogue of deoxycytidine triphosphate (dCTP) that is a substrate for a DNA polymerase. The term “dGTP analogue” refers to an analogue of deoxyguanosine triphosphate (dGTP) that is a substrate for a DNA polymerase. The term “dNTP analogue” refers to an analogue of deoxynucleoside triphosphate (dNTP) that is a substrate for a DNA polymerase. The term “dTTP analogue” refers to an analogue of deoxythymidine triphosphate (dUTP) that is a substrate for a DNA polymerase. The term “dUTP analogue” refers to an analogue of deoxyuridine triphosphate (dUTP) that is a substrate for a DNA polymerase.

The term “extendible” means, in the context of a nucleotide, primer, or extension product, that the 3′-OH group of the particular molecule is available and accessible to a DNA polymerase for extension or addition of nucleotides derived from dNTPs or dNTP analogues. “Incorporation” means joining of the modified nucleotide to the free 3′ hydroxyl group of a second nucleotide via formation of a phosphodiester linkage with the 5′ phosphate group of the modified nucleotide. The second nucleotide to which the modified nucleotide is joined will typically occur at the 3′ end of a polynucleotide chain. As used herein, the term “incorporating” or “chemically incorporating,” when used in reference to a primer and a nucleotide, refers to the process of joining the nucleotide to the primer or extension product thereof by formation of a phosphodiester bond. As used herein, the term “extension” or “elongation” is used in accordance with their plain and ordinary meanings and refer to synthesis by a polymerase of a new polynucleotide strand (i.e., an “extension strand”) complementary to a template strand by adding free nucleotides (e.g., dNTPs) from a reaction mixture that are complementary to the template in a 5′-to-3′ direction, including condensing a 5′-phosphate group of a dNTPs with a 3′-hydroxy group at the end of the nascent (elongating) DNA strand.

The term “modified nucleotide” refers to nucleotide or nucleotide analogue modified in some manner. Typically, a nucleotide contains a single 5-carbon sugar moiety, a single nitrogenous base moiety and 1 to three phosphate moieties. In particular, embodiments, a nucleotide can include a blocking moiety or a label moiety. A blocking moiety (e.g., a reversible terminator moiety) on a nucleotide prevents formation of a covalent bond between the 3′ hydroxyl moiety of the nucleotide and the 5′ phosphate of another nucleotide. A blocking moiety on a nucleotide can be reversible (i.e., a reversible terminator), whereby the blocking moiety can be removed or modified to allow the 3′ hydroxyl to form a covalent bond with the 5′ phosphate of another nucleotide. A blocking moiety can be effectively irreversible under particular conditions used in a method set forth herein. A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both.

A “removable” group, e.g., a label or a blocking group or protecting group, refers to a chemical group that can be removed from a dNTP analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a dNTP of dNTP analogue.

“Reversible blocking groups” or “reversible terminators” include a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator.

3′-O-Blocked Reversible Terminator

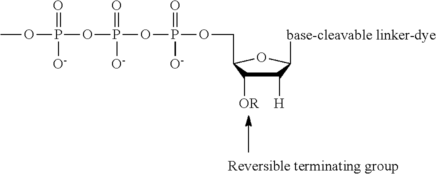

In embodiments, provided herein are polymerases capable of incorporating three differently sized reversible terminator probes linked to the 3′ oxygen: an A-Term, S-Term, and i-term. A-Term refers to azide-containing terminators (Guo J, et al. PNAS 2008); for example having the formula:

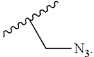 [SMILES: *CN=[N+]=[N-]]

S-Term refers to sulfide-containing terminators (WO 2017/058953); for example having the formula

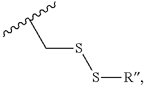

wherein R″ is unsubstituted C₁-C₄ alkyl. The i-Term probe refers to an isomeric reversible terminator For example, an i-term probe has the formula:

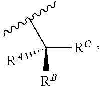

wherein R^{A} and R^{B} are hydrogen or alkyl, wherein at least one of R^{A} or R^{B} are hydrogen to yield a stereoisomeric probe, and R^{C} is the remainder of the reversible terminator.

In embodiments, the nucleotide is

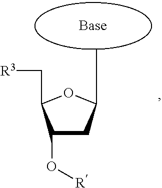

wherein Base is a Base as described herein, R³ is —OH, monophosphate, or polyphosphate or a nucleic acid, and R′ is a reversible terminator having the formula:

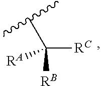

wherein R^{A} and R^{B} are hydrogen or alkyl and R^{C} is the remainder of the reversible terminator. In embodiments, the reversible terminator is

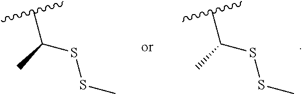

In embodiments, the reversible terminator is

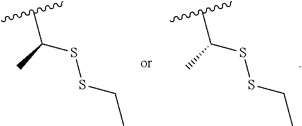

In embodiments, the reversible terminator is

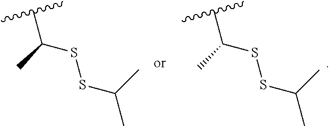

In embodiments, the reversible terminator is

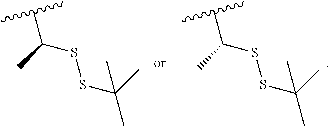

In embodiments, the reversible terminator is

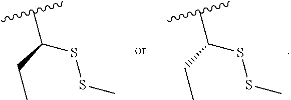

In embodiments, the reversible terminator is

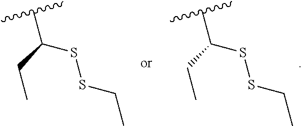

In embodiments, the reversible terminator is

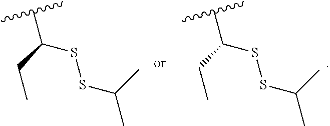

In embodiments, the reversible terminator is

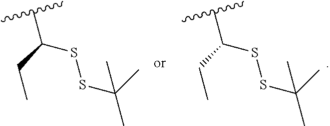

In embodiments, the reversible terminator is

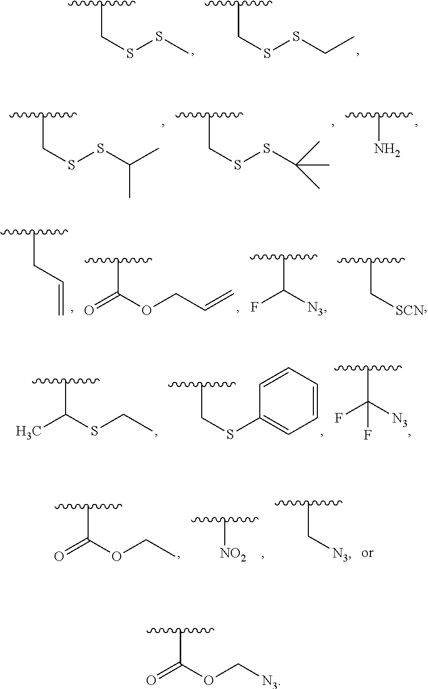

In nucleotides with 3′-unblocked reversible terminators, the termination group is linked to the base of the nucleotide as well as the label and functions not only as a reporter by as part of the reversible terminating group for termination of primer extension during sequencing. The 3′-unblocked reversible terminators are known in the art and include for example, the “virtual terminator” as described in U.S. Pat. No. 8,114,973 and the “Lightening terminator” as described in U.S. Pat. No. 10,041,115, the contents of which are incorporated herein by reference in their entirety.

3′-Unblocked Reversible Terminator

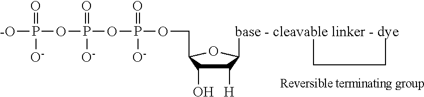

The term “non-covalent linker” is used in accordance with its ordinary meaning and refers to a divalent moiety which includes at least two molecules that are not covalently linked to each other but are capable of interacting with each other via a non-covalent bond (e.g. electrostatic interactions (e.g. ionic bond, hydrogen bond, halogen bond) or van der Waals interactions (e.g. dipole-dipole, dipole-induced dipole, London dispersion). In embodiments, the non-covalent linker is the result of two molecules that are not covalently linked to each other that interact with each other via a non-covalent bond.

The term “anchor moiety” as used herein refers to a chemical moiety capable of interacting (e.g., covalently or non-covalently) with a second, optionally different, chemical moiety (e.g., complementary anchor moiety binder). In embodiments, the anchor moiety is a bioconjugate reactive group capable of interacting (e.g., covalently) with a complementary bioconjugate reactive group (e.g., complementary anchor moiety reactive group). In embodiments, an anchor moiety is a click chemistry reactant moiety. In embodiments, the anchor moiety (an “affinity anchor moiety”) is capable of non-covalently interacting with a second chemical moiety (e.g., complementary affinity anchor moiety binder). Non-limiting examples of an anchor moiety include biotin, azide, trans-cyclooctene (TCO) and phenyl boric acid (PBA). In embodiments, an affinity anchor moiety (e.g., biotin moiety) interacts non-covalently with a complementary affinity anchor moiety binder (e.g., streptavidin moiety). In embodiments, an anchor moiety (e.g., azide moiety, trans-cyclooctene (TCO) moiety, phenyl boric acid (PBA) moiety) covalently binds a complementary anchor moiety binder (e.g., dibenzocyclooctyne (DBCO) moiety, tetrazine (TZ) moiety, salicylhydroxamic acid (SHA) moiety).

The terms “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), hydrazine (N₂H₄)). A chemically cleavable linker is non-enzymatically cleavable. In embodiments, the cleavable linker is cleaved by contacting the cleavable linker with a cleaving agent. In embodiments, the cleaving agent is sodium dithionite (Na₂S₂O₄), weak acid, hydrazine (N₂H₄), Pd(0), or light-irradiation (e.g., ultraviolet radiation).

A photocleavable linker (e.g., including or consisting of a o-nitrobenzyl group) refers to a linker which is capable of being split in response to photo-irradiation (e.g., ultraviolet radiation). An acid-cleavable linker refers to a linker which is capable of being split in response to a change in the pH (e.g., increased acidity). A base-cleavable linker refers to a linker which is capable of being split in response to a change in the pH (e.g., decreased acidity). An oxidant-cleavable linker refers to a linker which is capable of being split in response to the presence of an oxidizing agent. A reductant-cleavable linker refers to a linker which is capable of being split in response to the presence of an reducing agent (e.g., Tris(3-hydroxypropyl)phosphine). In embodiments, the cleavable linker is a dialkylketal linker, an azo linker, an allyl linker, a cyanoethyl linker, a 1-(4,4-dimethyl-2,6-dioxocyclohex ylidene)ethyl linker, or a nitrobenzyl linker.

The term “orthogonally cleavable linker” or “orthogonal cleavable linker” as used herein refer to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent. In embodiments, an orthogonally is a cleavable linker that following cleavage the two separated entities (e.g., fluorescent dye, bioconjugate reactive group) do not further react and form a new orthogonally cleavable linker.

The term “orthogonal binding group” or “orthogonal binding molecule” as used herein refer to a binding group (e.g. anchor moiety or complementary anchor moiety binder) that is capable of binding a first complementary binding group (e.g., complementary anchor moiety binder or anchor moiety) in a mixture of two or more different complementary binding groups and is unable to bind any other different complementary binding group in the mixture of two or more complementary binding groups. For example, two different binding groups are both orthogonal binding groups when a mixture of the two different binding groups are reacted with two complementary binding groups and each binding group binds only one of the complementary binding groups and not the other complementary binding group. An example of a set of four orthogonal binding groups and a set of orthogonal complementary binding groups are the binding groups biotin, azide, trans-cyclooctene (TCO) and phenyl boric acid (PBA), which specifically and efficiently bind or react with the complementary binding groups streptavidin, dibenzocyclooctyne (DBCO), tetrazine (TZ) and salicylhydroxamic acid (SHA) respectively.

The term “orthogonal detectable label” or “orthogonal detectable moiety” as used herein refer to a detectable label (e.g. fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye. Orthogonal detectable labels may be separately identified by different absorbance or emission intensities of the orthogonal detectable labels compared to each other and not only be the absolute presence of absence of a signal. An example of a set of four orthogonal detectable labels is the set of Rox-Labeled Tetrazine, Alexa488-Labeled SHA, Cy5-Labeled Streptavidin, and R6G-Labeled Dibenzocyclooctyne.

The term “polymerase-compatible cleavable moiety” as used herein refers a cleavable moiety which does not interfere with the function of a polymerase (e.g., DNA polymerase, modified DNA polymerase). Methods for determining the function of a polymerase contemplated herein are described in B. Rosenblum et al. (Nucleic Acids Res. 1997 Nov. 15; 25(22): 4500-4504); and Z. Zhu et al. (Nucleic Acids Res. 1994 Aug. 25; 22(16): 3418-3422), which are incorporated by reference herein in their entirety for all purposes. In embodiments the polymerase-compatible cleavable moiety does not decrease the function of a polymerase relative to the absence of the polymerase-compatible cleavable moiety. In embodiments, the polymerase-compatible cleavable moiety does not negatively affect DNA polymerase recognition. In embodiments, the polymerase-compatible cleavable moiety does not negatively affect (e.g., limit) the read length of the DNA polymerase. Additional examples of a polymerase-compatible cleavable moiety may be found in U.S. Pat. No. 6,664,079, Ju J. et al. (2006) Proc Natl Acad Sci USA 103(52):19635-19640; Ruparel H. et al. (2005) Proc Natl Acad Sci USA 102(17):5932-5937; Wu J. et al. (2007) Proc Natl Acad Sci USA 104(104):16462-16467; Guo J. et al. (2008) Proc Natl Acad Sci USA 105(27): 9145-9150 Bentley D. R. et al. (2008) Nature 456(7218):53-59; or Huffer D. et al. (2010) Nucleosides Nucleotides & Nucleic Acids 29:879-895, which are incorporated herein by reference in their entirety for all purposes. In embodiments, a polymerase-compatible cleavable moiety includes an azido moiety or a dithiol linking moiety. In embodiments, the polymerase-compatible cleavable moiety is —NH₂, —CN, —CH₃, C₂-C₆ allyl (e.g., —CH₂—CH═CH₂), methoxyalkyl (e.g., —CH₂—O—CH₃), or —CH₂N₃. In embodiments, the polymerase-compatible cleavable moiety comprises a disulfide moiety.**

A “detectable agent” or “detectable compound” or “detectable label” or “detectable moiety” is a composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴TC, ⁹⁴TC, ^{99m}Tc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescein isothiocyanate moiety, tetramethylrhodamine-5-(and 6)-isothiocyanate moiety, Cy2 moeity, Cy3 moiety, Cy5 moiety, Cy7 moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, the detectable label is a fluorescent dye. In embodiments, the detectable label is a fluorescent dye capable of exchanging energy with another fluorescent dye (e.g., fluorescence resonance energy transfer (FRET) chromophores).

The term “cyanine” or “cyanine moiety” as described herein refers to a detectable moiety containing two nitrogen groups separated by a polymethine chain. In embodiments, the cyanine moiety has 3 methine structures (i.e. cyanine 3 or Cy3). In embodiments, the cyanine moiety has 5 methine structures (i.e. cyanine 5 or Cy5). In embodiments, the cyanine moiety has 7 methine structures (i.e. cyanine 7 or Cy7).

Descriptions of nucleotide analogues of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following groups each contain amino acids that are conservative substitutions for one another: 1) Non-polar—Alanine (A), Leucine (L), Isoleucine (I), Valine (V), Glycine (G), Methionine (M); 2) Aliphatic—Alanine (A), Leucine (L), Isoleucine (I), Valine (V); 3) Acidic—Aspartic acid (D), Glutamic acid (E); 4) Polar—Asparagine (N), Glutamine (Q); Serine (S), Threonine (T); 5) Basic—Arginine (R), Lysine (K); 7) Aromatic—Phenylalanine (F), Tyrosine (Y), Tryptophan (W), Histidine (H); 8) Other—Cysteine (C) and Proline (P).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithm with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length. As used herein, percent (%) amino acid sequence identity is defined as the percentage of amino acids in a candidate sequence that is identical to the amino acids in a reference sequence, after aligning the sequences and introducing gaps, if necessary, to achieve the maximum percent sequence identity. Alignment for purposes of determining percent sequence identity can be achieved in various ways that are within the level of skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 700, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The term “DNA polymerase” and “nucleic acid polymerase” are used in accordance with their plain ordinary meaning and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol t DNA polymerase, Pol ν DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9° N polymerase (exo-), Therminator II, Therminator III, or Therminator IX). In embodiments, the DNA polymerase is a Pyrococcus DNA polymerase. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase

The term “thermophilic nucleic acid polymerase” as used herein refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase, including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L4085/Y409A/P410V mutations, NEB Therminator IX DNA polymerase), or γ-phosphate labeled nucleotides (e.g., Therminator γ: D141A/E143A/W355A/L408W/R460A/Q4615/K464E/D480V/R484W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. PNAS. 2008; 105(27):9145-9150), which are incorporated herein in their entirety for all purposes.

In the context of this application, the term “motif A region” specifically refers to the three amino acids functionally equivalent, corresponding to, positionally equivalent, or homologous to amino acids 409, 410, and 411 in wild type P. horikoshii; these amino acids are functionally equivalent to amino acid positions 408, 409, and 410 in 9° N polymerase. Functionally equivalent, positionally equivalent, or homologous “motif A regions” of polymerases other than P. horikoshii can be identified on the basis of amino acid sequence alignment and/or molecular modeling. Sequence alignments may be compiled using any of the standard alignment tools known in the art, such as for example BLAST, DIAMOND (Buchfink et al. Nat Methods 12, 59-60 (2015)), and the like.

The terms “position”, “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. Similarly, the term “functionally equivalent to” in relation to an amino acid position refers to an amino acid residue in a protein that corresponds to a particular amino acid in a reference sequence. An amino acid “corresponds” to a given residue when it occupies the same essential structural position within the protein as the given residue. One skilled in the art will immediately recognize the identity and location of residues corresponding to a specific position in a protein (e.g., polymerase) in other proteins with different numbering systems. For example, by performing a simple sequence alignment with a protein (e.g., polymerase) the identity and location of residues corresponding to specific positions of said protein are identified in other protein sequences aligning to said protein. For example, a selected residue in a selected protein corresponds to methionine at position 129 when the selected residue occupies the same essential spatial or other structural relationship as a methionine at position 129. In some embodiments, where a selected protein is aligned for maximum homology with a protein, the position in the aligned selected protein aligning with methionine 129 is said to correspond to methionine 129. Instead of a primary sequence alignment, a three-dimensional structural alignment can also be used, e.g., where the structure of the selected protein is aligned for maximum correspondence with the methionine at position 129, and the overall structures compared. In this case, an amino acid that occupies the same essential position as methionine 129 in the structural model is said to correspond to the methionine 129 residue. For example, references to a P. horikoshii polymerase amino acid position recited herein may refer to a numbered position set forth in SEQ ID NO:1, or the corresponding position in a polymerase homolog of SEQ ID NO:1. For example, when identifying an amino acid that corresponds to a position in SEQ ID NO:1, aligning the second enzyme species identifies if any insertions and/or deletions shift the position of the amino acid. An alignment of SEQ ID NO:3 to SEQ ID NO:1, a portion of which is provided in FIG. 2, highlights a deletion in SEQ ID NO:3 relative to SEQ ID NO:1, such that any amino acid positions beyond amino acid position 554 are shifted −1 in SEQ ID NO:3 relative to SEQ ID NO:1 (i.e., amino acid E554 in SEQ ID NO:3 corresponds to E555 in SEQ ID NO:1).

In embodiments, the polymerase may include an amino acid substitution mutation at a particular position corresponding to a position in SEQ ID NO: 1. For example, in embodiments, the polymerase includes an amino acid substitution mutation at position 141, which means the variant polymerase has a different amino acid at position 141 compared to SEQ ID NO: 1. In embodiments, the polymerase includes an amino acid substitution mutation at more than one position compared to SEQ ID NO: 1. For example, in embodiments, the polymerase includes the following substitution mutations: D141A; E143A; L4095; Y410A; P411V, where the number refers to the corresponding position in SEQ ID NO: 1. One having skill in the art would understand the amino acid mutation nomenclature, such that D141A refers to aspartic acid (single letter code is D), at position 141, being replaced with alanine (single letter code A).

The term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide, thereby releasing deoxyribonucleoside 5′-monophosphates one after another. One having skill in the art understands that an enzyme having 3′-5′ exonuclease activity does not cleave DNA strands without terminal 3′-OH moieties. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996).

The terms “measure”, “measuring”, “measurement” and the like refer not only to quantitative measurement of a particular variable, but also to qualitative and semi-quantitative measurements. Accordingly, “measurement” also includes detection, meaning that merely detecting a change, without quantification, constitutes measurement.

A “polymerase-template complex” refers to a functional complex between a DNA polymerase and a DNA primer-template molecule (e.g., nucleic acid). In embodiments, the polymerase is non-covalently bound to a nucleic acid primer and the template nucleic acid molecule.

The terms “sequencing”, “sequence determination”, “determining a nucleotide sequence”, and the like include determination of partial as well as full sequence information of the polynucleotide being sequenced. That is, the term includes sequence comparisons, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide.

The term “sequencing reaction mixture” refers to an aqueous mixture that contains the reagents necessary to allow a dNTP or dNTP analogue to add a nucleotide to a DNA strand by a DNA polymerase. Exemplary mixtures include buffers (e.g., saline-sodium citrate (SSC), tris(hydroxymethyl)aminomethane or “Tris”), salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), detergents and/or crowding agents or stabilizers (e.g., PEG, Tween, BSA).

The term “solid substrate” means any suitable medium present in the solid phase to which an antibody or an agent can be covalently or non-covalently affixed or immobilized. Preferred solid substrates are glass. Non-limiting examples include chips, beads and columns. The solid substrate can be non-porous or porous. Exemplary solid substrates include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides, etc.), nylon, ceramics, resins, Zeonor, silica or silica-based materials including silicon and modified silicon, carbon, metals, inorganic glasses, optical fiber bundles, and polymers.

The term “species”, when used in the context of describing a particular compound or molecule species, refers to a population of chemically indistinct molecules. When used in the context of taxonomy, “species” is the basic unit of classification and a taxonomic rank. For example, in reference to the microorganism Pyrococcus horikoshii, horikoshii is a species of the genus Pyrococcus.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

“Control” or “control experiment” is used in accordance with its plain ordinary meaning and refers to an experiment in which the subjects (e.g., enzymes) or reagents of the experiment are treated as in a parallel experiment except for omission of a procedure, reagent, or variable of the experiment (e.g., a polymerase not having one or more mutations relative to the polymerase being tested). In some instances, the control is used as a standard of comparison in evaluating experimental effects. In some embodiments, a control is the measurement of the activity of a protein in the absence of a mutation as described herein (including embodiments and examples). “Control polymerase” is defined herein as the polymerase against which the activity of the altered polymerase is compared. In one embodiment of the invention the control polymerase may comprise a wild type polymerase or an exo-variant thereof. Unless otherwise stated, by “wild type” it is generally meant that the polymerase comprises its natural amino acid sequence, as it would be found in nature. The invention is not limited to merely a comparison of activity of the polymerases as described herein against the wild type equivalent or exo-variant of the polymerase that is being altered. Many polymerases exist whose amino acid sequence has been modified (e.g., by amino acid substitution mutations) and which can prove to be a suitable control for use in assessing the modified nucleotide incorporation efficiencies of the polymerases as described herein. The control polymerase can, therefore, comprise any known polymerase, including mutant polymerases known in the art. The activity of the chosen “control” polymerase with respect to incorporation of the desired nucleotide analogues may be determined by an incorporation assay.

The term “modulate” is used in accordance with its plain ordinary meaning and refers to the act of changing or varying one or more properties. “Modulation” refers to the process of changing or varying one or more properties.

The term “kit” is used in accordance with its plain ordinary meaning and refers to any delivery system for delivering materials or reagents for carrying out a method of the invention. Such delivery systems include systems that allow for the storage, transport, or delivery of reaction reagents (e.g., nucleotides, enzymes, nucleic acid templates, etc. in the appropriate containers) and/or supporting materials (e.g., buffers, written instructions for performing the reaction, etc.) from one location to another location. For example, kits include one or more enclosures (e.g., boxes) containing the relevant reaction reagents and/or supporting materials. Such contents may be delivered to the intended recipient together or separately. For example, a first container may contain an enzyme, while a second container contains nucleotides. In embodiments, the kit includes vessels containing one or more enzymes, primers, adapters, or other reagents as described herein. Vessels may include any structure capable of supporting or containing a liquid or solid material and may include tubes, vials, jars, containers, tips, etc. In embodiments, a wall of a vessel may permit the transmission of light through the wall. In embodiments, the vessel may be optically clear. The kit may include the enzyme and/or nucleotides in a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The phrase “stringent hybridization conditions” refers to conditions under which a primer will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

“Synthetic” DNA polymerases refer to non-naturally occurring DNA polymerases such as those constructed by synthetic methods, mutated parent DNA polymerases such as truncated DNA polymerases and fusion DNA polymerases (e.g., as described in U.S. Pat. No. 7,541,170). Variants of the parent DNA polymerase have been engineered by mutating residues using site-directed or random mutagenesis methods known in the art. In embodiments, the mutations are in any of Motifs I-VI. The variant is expressed in an expression system such as E. coli by methods known in the art. The variant is then screened using the assays described herein to determine activity (e.g., strand-displacing activity).

As used herein, the term “template polynucleotide” or “template nucleic acid” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The terms “single strand” and “ssDNA” are used in accordance with its plain and ordinary meaning and refer to a single-stranded polynucleotide. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g. apoptosis or necrosis) or cell shedding, releasing polynucleotides into surrounding body fluids or into circulation. Accordingly, cell-free polynucleotides may be isolated from a non-cellular fraction of blood (e.g. serum or plasma), from other bodily fluids (e.g. urine), or from non-cellular fractions of other types of samples.

As used herein, a “native” nucleotide is used in accordance with its plain and ordinary meaning and refers to a naturally occurring nucleotide that does not include an exogenous label (e.g., a fluorescent dye, or other label) or chemical modification such as those that may characterize a nucleotide analog (e.g., a reversible terminating moiety). Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). A “canonical” nucleotide is an unmodified nucleotide.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

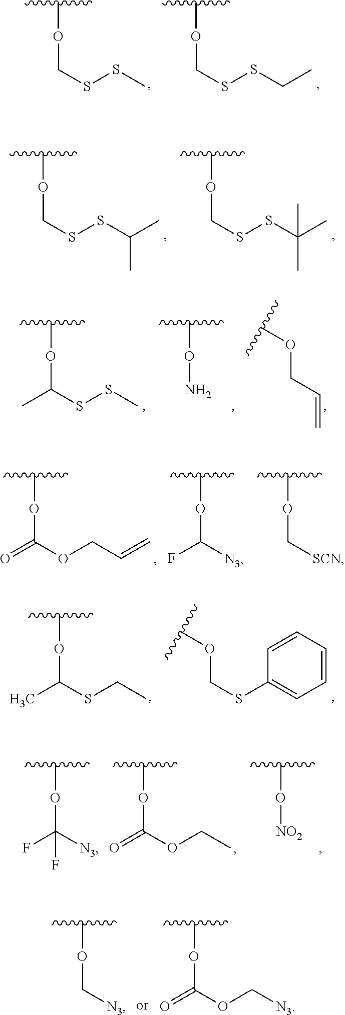

A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. Nos. 6,664,079, 10,738,072, and 11,174,281, each which are incorporated herein by reference in their entirety for all purposes.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. When referring to a double-stranded polynucleotide including a first strand hybridized to a second strand, it is to be understood that each of the terms “first strand” and “second strand” refer to single-stranded polynucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often comprise nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As used herein, the terms “solid support” and “substrate” and “solid surface” are used interchangeably and refers to discrete solid or semi-solid surfaces to which a plurality of nucleic acid (e.g., primers) may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further comprise a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface comprising a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate comprises a surface (e.g., a surface of a flow cell, a surface of a tube, a surface of a chip, surface of a particle), for example a metal surface (e.g., steel, gold, silver, aluminum, silicon and copper). In some embodiments a substrate (e.g., a substrate surface) is coated and/or comprises functional groups and/or inert materials. In certain embodiments a substrate comprises a bead, a chip, a capillary, a plate, a membrane, a wafer (e.g., silicon wafers), a comb, or a pin for example. In some embodiments a substrate comprises a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, silica, nylon, Wang resin, Merrifield resin, metal (e.g., iron, a metal alloy, sepharose, agarose, polyacrylamide, dextran, cellulose and the like or combinations thereof. In some embodiments a substrate comprises a magnetic material (e.g., iron, nickel, cobalt, platinum, aluminum, and the like). In certain embodiments a substrate comprises a magnetic bead (e.g., DYNABEADS®, hematite, AMPure XP). Magnets can be used to purify and/or capture nucleic acids bound to certain substrates (e.g., substrates comprising a metal or magnetic material).

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer. Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

The term “surface” is intended to mean an external part or external layer of a substrate. The surface can be in contact with another material such as a gas, liquid, gel, polymer, organic polymer, second surface of a similar or different material, metal, or coating. The surface, or regions thereof, can be substantially flat. The substrate and/or the surface can have surface features such as wells, pits, channels, ridges, raised regions, pegs, posts or the like.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers comprising tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyperbranched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as a polymerase described herein, including embodiments. RCA may be performed by using any of the DNA polymerases that are known in the art (e.g., a Phi29 DNA polymerase, a Bst DNA polymerase, or SD polymerase).

As used herein, the term “hybridize” or “specifically hybridize” refers to a process where two complementary nucleic acid strands anneal to each other under appropriately stringent conditions. Hybridizations are typically and preferably conducted with oligonucleotides. The terms “annealing” and “hybridization” are used interchangeably to mean the formation of a stable duplex. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. Those skilled in the art understand how to estimate and adjust the stringency of hybridization conditions such that sequences having at least a desired level of complementarity will stably hybridize, while those having lower complementarity will not. As used herein, the term “stringent condition” refers to condition(s) under which a polynucleotide probe or primer will hybridize preferentially to its target sequence, and to a lesser extent to, or not at all to, other sequences. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands (e.g., two single-stranded polynucleotides) that are hybridized to each other can form a duplex which comprises a double-stranded portion of nucleic acid.

II. Polymerase, Complexes, and Kits

Provided herein are, inter alia, modified Pyrococcus Family B DNA polymerases. Family B polymerases characteristically have separate domains for DNA polymerase activity and 3′-5′ exonuclease activity. The exonuclease domain is characterized by as many as six and at least three conserved amino acid sequence motifs in and around a structural binding pocket. During polymerization, nucleotides are added to the 3′ end of the primer strand and during the 3′-5′ exonuclease reaction, the 3′ terminus of the primer is shifted to the 3′-5′ exonuclease domain and the one or more of the 3′-terminal nucleotides are hydrolyzed. In embodiments, the variants of a Pyrococcus family B DNA polymerase provided herein have detectable strand displacing activity and are useful in methods of incorporating modified nucleotides in nucleic acid synthesis reactions. In embodiments, the polymerase is a thermophilic nucleic acid polymerase.

In an aspect is provided a polymerase including an amino acid sequence that is at least 80% identical to a continuous 500 amino acid sequence within SEQ ID NO: 1; wherein the polymerase includes a first mutation at amino acid position 409 or an amino acid position corresponding to position 409; and at least one mutation at amino acid position 7 or an amino acid position corresponding to position 7, wherein the mutation at amino acid position 7 includes histidine, lysine, or arginine; amino acid position 579 or an amino acid position corresponding to position 579, wherein the mutation at amino acid position 579 includes leucine, isoleucine, valine, alanine, or glycine; amino acid position 588 or an amino acid position corresponding to position 588, wherein the mutation at amino acid position 588 includes leucine, isoleucine, valine, alanine, or glycine; or amino acid position 742 or an amino acid position corresponding to position 742, wherein the mutation at amino acid position 742 includes leucine, isoleucine, alanine, or glycine.

In an aspect is provided a polymerase including an amino acid sequence that is at least 80% identical to a continuous 500 amino acid sequence within SEQ ID NO: 1; including a first mutation at amino acid position 409 or an amino acid position corresponding to position 409; and at least one mutation at amino acid position 7 or an amino acid position corresponding to position 7; at amino acid position 579 or an amino acid position corresponding to position 579; at amino acid position 588 or an amino acid position corresponding to position 588; or at amino acid position 742 or an amino acid position corresponding to position 742.

In embodiments, the variants of a Pyrococcus family B DNA polymerase are derived from a Pyrococcus species. In embodiments, the Pyrococcus species include Pyrococcus abyssi, Pyrococcus endeavors, Pyrococcus furiosus, Pyrococcus glycovorans, Pyrococcus horikoshii, Pyrococcus kukulkanii, Pyrococcus woesei, Pyrococcus yayanosii, Pyrococcus sp., Pyrococcus sp. 12/1, Pyrococcus sp. 121, Pyrococcus sp. 303, Pyrococcus sp. 304, Pyrococcus sp. 312, Pyrococcus sp. 32-4, Pyrococcus sp. 321, Pyrococcus sp. 322, Pyrococcus sp. 323, Pyrococcus sp. 324, Pyrococcus sp. 95-12-1, Pyrococcus sp. AV5, Pyrococcus sp. Ax99-7, Pyrococcus sp. C2, Pyrococcus sp. EX2, Pyrococcus sp. Fla95-Pc, Pyrococcus sp. GB-3A, Pyrococcus sp. GB-D, Pyrococcus sp. GBD, Pyrococcus sp. GI-H, Pyrococcus sp. GI-J, Pyrococcus sp. GIL, Pyrococcus sp. HT3, Pyrococcus sp. JT1, Pyrococcus sp. LMO-A29, Pyrococcus sp. LMO-A30, Pyrococcus sp. LMO-A31, Pyrococcus sp. LMO-A32, Pyrococcus sp. LMO-A33, Pyrococcus sp. LMO-A34, Pyrococcus sp. LMO-A35, Pyrococcus sp. LMO-A36, Pyrococcus sp. LMO-A37, Pyrococcus sp. LMO-A38, Pyrococcus sp. LMO-A39, Pyrococcus sp. LMO-A40, Pyrococcus sp. LMO-A41, Pyrococcus sp. LMO-A42, Pyrococcus sp. M24D13, Pyrococcus sp. MA2.31, Pyrococcus sp. MA2.32, Pyrococcus sp. MA2.34, Pyrococcus sp. MV1019, Pyrococcus sp. MV4, Pyrococcus sp. MV7, Pyrococcus sp. MZ14, Pyrococcus sp. MZ4, Pyrococcus sp. NA2, Pyrococcus sp. NS102-T, Pyrococcus sp. P12.1, Pyrococcus sp. Pikanate 5017, Pyrococcus sp. PK 5017, Pyrococcus sp. ST04, Pyrococcus sp. ST700, Pyrococcus sp. Tc-2-70, Pyrococcus sp. Tc95-7C-I, Pyrococcus sp. TC95-7C-S, Pyrococcus sp. Tc95_6, Pyrococcus sp. V211, Pyrococcus sp. V212, Pyrococcus sp. V221, Pyrococcus sp. V222, Pyrococcus sp. V231, Pyrococcus sp. V232, Pyrococcus sp. V61, Pyrococcus sp. V62, Pyrococcus sp. V63, Pyrococcus sp. V72, Pyrococcus sp. V73, Pyrococcus sp. VB112, Pyrococcus sp. VB113, Pyrococcus sp. VB81, Pyrococcus sp. VB82, Pyrococcus sp. VB83, Pyrococcus sp. VB85, Pyrococcus sp. VB86, Pyrococcus sp. VB93 polymerase, Pyrococcus furiosus DSM 3638, Pyrococcus sp. GE23, Pyrococcus sp. GI-H, Pyrococcus sp. NA2, Pyrococcus sp. ST04, or Pyrococcus sp. ST700. In embodiments, the variants of a Pyrococcus family B DNA polymerase provided herein are a Pyrococcus horikoshii family B DNA polymerase that have strand-displacing activity and are useful in methods of incorporating modified nucleotides in nucleic acid synthesis reactions. In embodiments, the variants of a Pyrococcus family B DNA polymerase provided herein are a Pyrococcus abyssi family B DNA polymerase that have strand-displacing activity and are useful in methods of incorporating modified nucleotides in nucleic acid synthesis reactions.

Parent archaeal polymerases may be DNA polymerases that are isolated from naturally occurring organisms. The parent DNA polymerases, also referred to as wild type polymerase, share the property of having a structural binding pocket that binds and hydrolyzes a substrate nucleic acid, producing 5′-dNMP. The structural binding pocket in this family of polymerases also shares the property of having sequence motifs that form the binding pocket, referred to as Exo Motifs I-VI. In embodiments, the parent or wild type P. horikoshii polymerase has an amino acid sequence comprising SEQ ID NO: 1. In embodiments, the polymerase has one or more amino acid substitution mutations relative to SEQ ID NO: 1.

In embodiments, the polymerase (a synthetic or variant DNA polymerase) provided herein may contain 1, 2, 3, 4, 5, 6, 7, 8, 9, 10, 11, 12, 13, 14, 15, or more mutations as compared to the wild-type sequence of SEQ ID NO: 1. The polymerase (a synthetic or variant DNA polymerase) may contain 10, 20, 30, 40, 50 or more mutations as compared to the wild-type sequence of SEQ ID NO: 1. The polymerase (a synthetic or variant DNA polymerase) may contain between 10 and 20 (inclusive of endpoints, e.g., 10, 41 . . . 49, and 20), between 20 and 30, between 30 and 40, or between 40 or 50 mutations as compared to SEQ ID NO: 1.

In embodiments, the polymerase includes an amino acid sequence that is at least 85% identical to a continuous 500 amino acid sequence within SEQ ID NO: 1. In embodiments, the polymerase includes an amino acid sequence that is at least 90% identical to a continuous 500 amino acid sequence within SEQ ID NO: 1. In embodiments, the polymerase includes an amino acid sequence that is at least 95% identical to a continuous 500 amino acid sequence within SEQ ID NO: 1. In embodiments, the polymerase includes an amino acid sequence that is at least 98% identical to a continuous 500 amino acid sequence within SEQ ID NO: 1. In embodiments, the polymerase includes an amino acid sequence that is at least 99% identical to a continuous 500 amino acid sequence within SEQ ID NO: 1. In embodiments, the polymerase includes an amino acid sequence that is 90% identical to a continuous 500 amino acid sequence within SEQ ID NO: 1. In embodiments, the polymerase includes an amino acid sequence that is 95% identical to a continuous 500 amino acid sequence within SEQ ID NO: 1. In embodiments, the polymerase includes an amino acid sequence that is 90% identical to SEQ ID NO: 1. In embodiments, the polymerase includes an amino acid sequence that is 95% identical to SEQ ID NO: 1.

In embodiments, the polymerase includes an amino acid sequence that is at least 80% identical to a continuous 500 amino acid sequence within SEQ ID NO: 1; including a first mutation at amino acid position 409; and at least one mutation at amino acid position 7, wherein the mutation at amino acid position 7 includes histidine, lysine, or arginine; amino acid position 579, wherein the mutation at amino acid position 579 includes leucine, isoleucine, valine, alanine, or glycine; amino acid position 588, wherein the mutation at amino acid position 588 includes leucine, isoleucine, valine, alanine, or glycine; or amino acid position 742, wherein the mutation at amino acid position 742 includes leucine, isoleucine, alanine, or glycine.

In embodiments, the first mutation at amino acid position 409 or the amino acid position corresponding to position 409 is alanine, glutamine, tyrosine, phenylalanine, isoleucine, valine, cysteine, serine, or histidine. In embodiments, the first mutation at amino acid position 409 is alanine, glutamine, tyrosine, phenylalanine, isoleucine, valine, cysteine, serine, or histidine. In embodiments, the first mutation at amino acid position 409 or the amino acid position corresponding to position 409 is alanine. In embodiments, the first mutation at amino acid position 409 is alanine. In embodiments, the first mutation at amino acid position 409 or the amino acid position corresponding to position 409 is glutamine. In embodiments, the first mutation at amino acid position 409 is glutamine. In embodiments, the first mutation at amino acid position 409 or the amino acid position corresponding to position 409 is tyrosine. In embodiments, the first mutation at amino acid position 409 is tyrosine. In embodiments, the first mutation at amino acid position 409 or the amino acid position corresponding to position 409 is phenylalanine. In embodiments, the first mutation at amino acid position 409 is phenylalanine. In embodiments, the first mutation at amino acid position 409 or the amino acid position corresponding to position 409 is isoleucine. In embodiments, the first mutation at amino acid position 409 is isoleucine. In embodiments, the first mutation at amino acid position 409 or the amino acid position corresponding to position 409 is valine. In embodiments, the first mutation at amino acid position 409 is valine. In embodiments, the first mutation at amino acid position 409 or the amino acid position corresponding to position 409 is cysteine. In embodiments, the first mutation at amino acid position 409 is cysteine. In embodiments, the first mutation at amino acid position 409 or the amino acid position corresponding to position 409 is serine. In embodiments, the first mutation at amino acid position 409 is serine. In embodiments, the first mutation at amino acid position 409 or the amino acid position corresponding to position 409 is histidine. In embodiments, the first mutation at amino acid position 409 is histidine.

In embodiments, the polymerase includes a glycine or alanine at amino acid position 410 or an amino acid position corresponding to position 410. In embodiments, the polymerase includes a glycine or alanine at amino acid position 410. In embodiments, the polymerase includes a glycine at amino acid position 410 or an amino acid position corresponding to position 410. In embodiments, the polymerase includes a glycine at amino acid position 410. In embodiments, the polymerase includes an alanine at amino acid position 410 or an amino acid position corresponding to position 410. In embodiments, the polymerase includes an alanine at amino acid position 410.

In embodiments, the polymerase includes a proline, serine, alanine, glycine, valine, or isoleucine at amino acid position 411 or an amino acid position corresponding to position 411. In embodiments, the polymerase includes a proline, serine, alanine, glycine, valine, or isoleucine at amino acid position 411. In embodiments, the polymerase includes a proline at amino acid position 411 or an amino acid position corresponding to position 411. In embodiments, the polymerase includes a proline at amino acid position 411. In embodiments, the polymerase includes a serine at amino acid position 411 or an amino acid position corresponding to position 411. In embodiments, the polymerase includes a serine at amino acid position 411. In embodiments, the polymerase includes an alanine at amino acid position 411 or an amino acid position corresponding to position 411. In embodiments, the polymerase includes an alanine at amino acid position 41. In embodiments, the polymerase includes a glycine at amino acid position 411 or an amino acid position corresponding to position 411. In embodiments, the polymerase includes a glycine at amino acid position 411. In embodiments, the polymerase includes a valine at amino acid position 411 or an amino acid position corresponding to position 411. In embodiments, the polymerase includes a valine at amino acid position 41. In embodiments, the polymerase includes an isoleucine at amino acid position 411 or an amino acid position corresponding to position 411. In embodiments, the polymerase includes an isoleucine at amino acid position 411.

In embodiments, the polymerase includes an amino acid substitution at position 409. The amino acid substitution at position 409 may be a serine substitution or an alanine substitution. In embodiments, the amino acid substitution at position 409 is a serine substitution. In embodiments, the amino acid substitution at position 409 is an alanine substitution. The amino acid substitution at position 409 may be a serine, cysteine, alanine, glycine, valine, isoleucine, glutamine, or histidine substitution. The amino acid substitution at position 409 may be a alanine, glycine, valine, isoleucine, threonine, glutamine, or histidine substitution.

In embodiments, the polymerase includes an amino acid substitution at position 410. The amino acid substitution at position 410 may be a glycine substitution or an alanine substitution. In embodiments, the amino acid substitution at position 410 is a glycine substitution. In embodiments, the amino acid substitution at position 410 is an alanine substitution. In embodiments, the amino acid substitution at position 410 is a valine substitution. In embodiments, the amino acid substitution at position 410 is a serine substitution. In embodiments, the amino acid substitution at position 410 is a proline substitution.

In embodiments, the polymerase includes an amino acid substitution at position 411. The amino acid substitution at position 411 may be an isoleucine substitution, a proline, a glycine substitution, a valine substitution, or a serine substitution. In embodiments, the amino acid substitution at position 411 is an isoleucine substitution. In embodiments, the amino acid substitution at position 411 is a proline. In embodiments, the amino acid substitution at position 411 is a glycine substitution. In embodiments, the amino acid substitution at position 411 is a valine substitution. In embodiments, the amino acid substitution at position 411 is a serine substitution. The amino acid substitution at position 411 may be glycine, alanine, leucine, isoleucine, proline, valine, leucine, serine, or threonine substitution. In embodiments, the amino acid substitution is a proline, alanine, or valine.

In embodiments, the polymerase includes an alanine or serine at amino acid position 409 or the amino acid position corresponding to position 409; a glycine at amino acid position 410 or an amino acid position corresponding to position 410; and a proline, valine, glycine, isoleucine, or serine at amino acid position 411 or an amino acid position corresponding to position 411. In embodiments, the polymerase includes an alanine or serine at amino acid position 409; a glycine at amino acid position 410; and a proline, valine, glycine, isoleucine, or serine at amino acid position 411. In embodiments, the polymerase includes an alanine at amino acid position 409 or the amino acid position corresponding to position 409. In embodiments, the polymerase includes an alanine at amino acid position 409. In embodiments, the polymerase includes a serine at amino acid position 409 or the amino acid position corresponding to position 409. In embodiments, the polymerase includes a serine at amino acid position 409. In embodiments, the polymerase includes a glycine at amino acid position 410 or the amino acid position corresponding to position 410. In embodiments, the polymerase includes a glycine at amino acid position 410. In embodiments, the polymerase includes a proline at amino acid position 411 or the amino acid position corresponding to position 411. In embodiments, the polymerase includes a proline at amino acid position 411. In embodiments, the polymerase includes a valine at amino acid position 411 or the amino acid position corresponding to position 411. In embodiments, the polymerase includes a valine at amino acid position 411. In embodiments, the polymerase includes a glycine at amino acid position 411 or the amino acid position corresponding to position 411. In embodiments, the polymerase includes a glycine at amino acid position 411. In embodiments, the polymerase includes an isoleucine at amino acid position 411 or the amino acid position corresponding to position 411. In embodiments, the polymerase includes an isoleucine at amino acid position 411. In embodiments, the polymerase includes a serine at amino acid position 411 or the amino acid position corresponding to position 411. In embodiments, the polymerase includes a serine at amino acid position 411. In embodiments, the polymerase includes an alanine at amino acid position 409 or the amino acid position corresponding to 409; a glycine at amino acid position 410 or the amino acid position corresponding to 410; and a proline at amino acid position 411 or the amino acid position corresponding to 411. In embodiments, the polymerase includes an alanine at amino acid position 409; a glycine at amino acid position 410; and a proline at amino acid position 411.

In embodiments, the mutation at amino acid position 588 or the amino acid corresponding to position 588 is leucine, isoleucine, valine, alanine, or glycine. In embodiments, the mutation at amino acid position 588 is leucine, isoleucine, valine, alanine, or glycine. In embodiments, the mutation at amino acid position 588 or the amino acid corresponding to position 588 is leucine. In embodiments, the mutation at amino acid position 588 is leucine. In embodiments, the mutation at amino acid position 588 or the amino acid corresponding to position 588 is isoleucine. In embodiments, the mutation at amino acid position 588 is isoleucine. In embodiments, the mutation at amino acid position 588 or the amino acid corresponding to position 588 is valine. In embodiments, the mutation at amino acid position 588 is valine. In embodiments, the mutation at amino acid position 588 or the amino acid corresponding to position 588 is alanine. In embodiments, the mutation at amino acid position 588 is alanine. In embodiments, the mutation at amino acid position 588 or the amino acid corresponding to position 588 is glycine. In embodiments, the mutation at amino acid position 588 is glycine.

In embodiments, the mutation at amino acid position 588 or the amino acid position corresponding to position 588 is leucine, isoleucine, or valine. In embodiments, the mutation at amino acid position 588 is leucine, isoleucine, or valine. In embodiments, the mutation at amino acid position 588 or the amino acid position corresponding to position 588 is isoleucine. In embodiments, the mutation at amino acid position 588 is isoleucine. In embodiments, the mutation at amino acid position 588 or the amino acid position corresponding to position 588 is valine. In embodiments, the mutation at amino acid position 588 is valine.

In embodiments, the mutation at amino acid position 7 or the amino acid position corresponding to position 7 is histidine, lysine, or arginine. In embodiments, the mutation at amino acid position 7 is histidine, lysine, or arginine. In embodiments, the mutation at amino acid position 7 or the amino acid position corresponding to position 7 is histidine. In embodiments, the mutation at amino acid position 7 is histidine. In embodiments, the mutation at amino acid position 7 or the amino acid position corresponding to position 7 is lysine. In embodiments, the mutation at amino acid position 7 is lysine. In embodiments, the mutation at amino acid position 7 or the amino acid position corresponding to position 7 is arginine. In embodiments, the mutation at amino acid position 7 is arginine. In embodiments, the mutation at amino acid position 7 or the amino acid position corresponding to position 7 is histidine. In embodiments, the mutation at amino acid position 7 is histidine. In embodiments, the polymerase includes a histidine, lysine, or arginine at amino acid position 7 and a mutation at amino acid position 97 (e.g., a cysteine or histidine at amino acid position 97. In embodiments, the polymerase includes a mutation at amino acid position 7 or an amino acid position corresponding to position 7, wherein the mutation is histidine, lysine, or arginine. In embodiments, the polymerase includes a mutation at amino acid position 7 or an amino acid position corresponding to position 7, wherein the mutation is histidine. In embodiments, the polymerase includes a mutation at amino acid position 7 or an amino acid position corresponding to position 7, wherein the mutation is lysine. In embodiments, the polymerase includes a mutation at amino acid position 7 or an amino acid position corresponding to position 7, wherein the mutation is arginine.

In embodiments, the mutation at amino acid position 13 or the amino acid position corresponding to position 13 is arginine or histidine. In embodiments, the mutation at amino acid position 13 is arginine or histidine. In embodiments, the mutation at amino acid position 13 or the amino acid position corresponding to position 13 is arginine. In embodiments, the mutation at amino acid position 13 is arginine. In embodiments, the mutation at amino acid position 13 or the amino acid position corresponding to position 13 is histidine. In embodiments, the mutation at amino acid position 13 is histidine.

In embodiments, the polymerase includes a mutation at amino acid position 13 or an amino acid position corresponding to position 13, wherein the mutation is arginine, isoleucine, methionine, or histidine. In embodiments, the polymerase includes a mutation at amino acid position 13 or an amino acid position corresponding to position 13, wherein the mutation is arginine or histidine. In embodiments, the polymerase includes a mutation at amino acid position 13 or an amino acid position corresponding to position 13, wherein the mutation is arginine. In embodiments, the polymerase includes a mutation at amino acid position 13 or an amino acid position corresponding to position 13, wherein the mutation is histidine. In embodiments, the polymerase includes a mutation at amino acid position 13 or an amino acid position corresponding to position 13, wherein the mutation is methionine. In embodiments, the polymerase includes a mutation at amino acid position 13 or an amino acid position corresponding to position 13, wherein the mutation is isoleucine. In embodiments, the polymerase includes a mutation at amino acid position 13 or an amino acid position corresponding to position 13, wherein the mutation is arginine, leucine, isoleucine, or histidine.

In embodiments, the polymerase includes a mutation at amino acid position 36 or an amino acid position corresponding to position 36, wherein the mutation is arginine, asparagine, serine, glutamic acid, glutamine, or histidine. In embodiments, the mutation at amino acid position 36 is arginine. In embodiments, the mutation at amino acid position 36 is asparagine. In embodiments, the mutation at amino acid position 36 is serine. In embodiments, the mutation at amino acid position 36 is glutamic acid. In embodiments, the mutation at amino acid position 36 is glutamine. In embodiments, the mutation at amino acid position 36 is histidine.

In embodiments, the polymerase includes a glutamine, valine, arginine, or alanine at amino acid position 80 or an amino acid position corresponding to position 80. In embodiments, the mutation at amino acid position 80 is glutamine, valine, arginine, or alanine. In embodiments, the mutation at amino acid position 80 is glutamine. In embodiments, the mutation at amino acid position 80 is valine. In embodiments, the mutation at amino acid position 80 is arginine. In embodiments, the mutation at amino acid position 80 is alanine.

In embodiments, the polymerase includes a mutation at amino acid position 91 or an amino acid position corresponding to position 91, wherein the mutation is histidine, lysine, or arginine. In embodiments, the polymerase includes a mutation at amino acid position 91 or an amino acid position corresponding to position 91, wherein the mutation is histidine. In embodiments, the polymerase includes a mutation at amino acid position 91 or an amino acid position corresponding to position 91, wherein the mutation is lysine. In embodiments, the polymerase includes a mutation at amino acid position 91 or an amino acid position corresponding to position 91, wherein the mutation is arginine.

In embodiments, the polymerase includes a glutamine, valine, arginine, or alanine at amino acid position 93 or an amino acid position corresponding to position 93. In embodiments, the mutation at amino acid position 93 is glutamine, valine, arginine, or alanine. In embodiments, the mutation at amino acid position 93 is glutamine. In embodiments, the mutation at amino acid position 93 is valine. In embodiments, the mutation at amino acid position 93 is arginine. In embodiments, the mutation at amino acid position 93 is alanine.

In embodiments, the mutation at amino acid position 97 or the amino acid position corresponding to position 97 is cysteine, histidine, lysine, serine, threonine, or methionine. In embodiments, the mutation at amino acid position 97 is cysteine, histidine, lysine, serine, threonine, or methionine. In embodiments, the mutation at amino acid position 97 or the amino acid position corresponding to position 97 is cysteine. In embodiments, the mutation at amino acid position 97 is cysteine. In embodiments, the mutation at amino acid position 97 or the amino acid position corresponding to position 97 is histidine. In embodiments, the mutation at amino acid position 97 is histidine. In embodiments, the mutation at amino acid position 97 or the amino acid position corresponding to position 97 is lysine. In embodiments, the mutation at amino acid position 97 is lysine. In embodiments, the mutation at amino acid position 97 or the amino acid position corresponding to position 97 is serine. In embodiments, the mutation at amino acid position 97 is serine. In embodiments, the mutation at amino acid position 97 or the amino acid position corresponding to position 97 is threonine. In embodiments, the mutation at amino acid position 97 is threonine. In embodiments, the mutation at amino acid position 97 or the amino acid position corresponding to position 97 is methionine. In embodiments, the mutation at amino acid position 97 is methionine.

In embodiments, the polymerase includes a mutation at amino acid position 97 or an amino acid position corresponding to position 97, wherein the mutation at amino acid position 97 is cysteine, histidine, leucine, lysine, serine, threonine, or methionine. In embodiments, the mutation at amino acid position 97 is cysteine. In embodiments, the mutation at amino acid position 97 is histidine. In embodiments, the mutation at amino acid position 97 is lysine. In embodiments, the mutation at amino acid position 97 is serine. In embodiments, the mutation at amino acid position 97 is threonine. In embodiments, the mutation at amino acid position 97 is methionine. In embodiments, the mutation at amino acid position 97 is leucine.

In embodiments, the polymerase includes a mutation at amino acid position 192 or an amino acid position corresponding to position 192, wherein the mutation is arginine, glutamic acid, methionine, or histidine. In embodiments, the mutation at amino acid position 192 is arginine. In embodiments, the mutation at amino acid position 192 is glutamic acid. In embodiments, the mutation at amino acid position 192 is methionine. In embodiments, the mutation at amino acid position 192 is histidine.

In embodiments, the mutation at amino acid position 241 or the amino acid position corresponding to position 241 is leucine, isoleucine, alanine, valine, or glycine. In embodiments, the mutation at amino acid position 241 is leucine, isoleucine, alanine, valine, or glycine. In embodiments, the mutation at amino acid position 241 or the amino acid position corresponding to position 241 is leucine. In embodiments, the mutation at amino acid position 241 is leucine. In embodiments, the mutation at amino acid position 241 or the amino acid position corresponding to position 241 is isoleucine. In embodiments, the mutation at amino acid position 241 is isoleucine. In embodiments, the mutation at amino acid position 241 or the amino acid position corresponding to position 241 is alanine. In embodiments, the mutation at amino acid position 241 is alanine. In embodiments, the mutation at amino acid position 241 or the amino acid position corresponding to position 241 is valine. In embodiments, the mutation at amino acid position 241 is valine. In embodiments, the mutation at amino acid position 241 or the amino acid position corresponding to position 241 is glycine. In embodiments, the mutation at amino acid position 241 is glycine.

In embodiments, the polymerase includes a mutation at amino acid position 245 or an amino acid position corresponding to position 245, wherein the mutation is threonine, serine, cysteine, or methionine. In embodiments, the polymerase includes a mutation at amino acid position 245 or an amino acid position corresponding to position 245, wherein the mutation is threonine. In embodiments, the polymerase includes a mutation at amino acid position 245 or an amino acid position corresponding to position 245, wherein the mutation is serine. In embodiments, the polymerase includes a mutation at amino acid position 245 or an amino acid position corresponding to position 245, wherein the mutation is cysteine. In embodiments, the polymerase includes a mutation at amino acid position 245 or an amino acid position corresponding to position 245, wherein the mutation is methionine.

In embodiments, the polymerase includes a mutation at amino acid position 246 or an amino acid position corresponding to amino acid position 246, wherein the mutation is glutamic acid, glycine, asparagine, or glutamine. In embodiments, the mutation at amino acid position 246 is glutamic acid. In embodiments, the mutation at amino acid position 246 is glycine. In embodiments, the mutation at amino acid position 246 is asparagine. In embodiments, the mutation at amino acid position 246 is glutamine.

In embodiments, the polymerase includes a mutation at amino acid position 465 or an amino acid position corresponding to position 465, wherein the mutation is asparagine, aspartic acid, glutamic acid, threonine, or glutamine. In embodiments, the polymerase includes a mutation at amino acid position 465 or an amino acid position corresponding to position 465, wherein the mutation is asparagine. In embodiments, the polymerase includes a mutation at amino acid position 465 or an amino acid position corresponding to position 465, wherein the mutation is aspartic acid. In embodiments, the polymerase includes a mutation at amino acid position 465 or an amino acid position corresponding to position 465, wherein the mutation is glutamic acid. In embodiments, the polymerase includes a mutation at amino acid position 465 or an amino acid position corresponding to position 465, wherein the mutation is glutamine. In embodiments, the polymerase includes a mutation at amino acid position 465 or an amino acid position corresponding to position 465, wherein the mutation is threonine.

In embodiments, the polymerase includes a mutation at amino acid position 467 or an amino acid position corresponding to position 467, wherein the mutation is asparagine, aspartic acid, serine, glutamic acid, threonine, or glutamine. In embodiments, the polymerase includes a mutation at amino acid position 467 or an amino acid position corresponding to position 467, wherein the mutation is asparagine. In embodiments, the polymerase includes a mutation at amino acid position 467 or an amino acid position corresponding to position 467, wherein the mutation is aspartic acid. In embodiments, the polymerase includes a mutation at amino acid position 467 or an amino acid position corresponding to position 467, wherein the mutation is glutamic acid. In embodiments, the polymerase includes a mutation at amino acid position 467 or an amino acid position corresponding to position 467, wherein the mutation is glutamine. In embodiments, the polymerase includes a mutation at amino acid position 467 or an amino acid position corresponding to position 467, wherein the mutation is threonine. In embodiments, the polymerase includes a mutation at amino acid position 467 or an amino acid position corresponding to position 467, wherein the mutation is serine.

In embodiments, the polymerase further includes a mutation at amino acid position 469 or the amino acid position corresponding to position 469, wherein the mutation is threonine, serine, cysteine, or methionine. In embodiments, the polymerase further includes a mutation at amino acid position 469, wherein the mutation is threonine, serine, cysteine, or methionine. In embodiments, the polymerase further includes a mutation at amino acid position 469 or the amino acid position corresponding to position 469, wherein the mutation is threonine. In embodiments, the polymerase further includes a mutation at amino acid position 469, wherein the mutation is threonine. In embodiments, the polymerase further includes a mutation at amino acid position 469 or the amino acid position corresponding to position 469, wherein the mutation is serine. In embodiments, the polymerase further includes a mutation at amino acid position 469, wherein the mutation is serine. In embodiments, the polymerase further includes a mutation at amino acid position 469 or the amino acid position corresponding to position 469, wherein the mutation is cysteine. In embodiments, the polymerase further includes a mutation at amino acid position 469, wherein the mutation is cysteine. In embodiments, the polymerase further includes a mutation at amino acid position 469 or the amino acid position corresponding to position 469, wherein the mutation is methionine. In embodiments, the polymerase further includes a mutation at amino acid position 469, wherein the mutation is methionine.

In embodiments, the polymerase further includes a mutation at amino acid position 469 or the amino acid position corresponding to position 469, wherein the mutation is asparagine, aspartic acid, glutamic acid, or glutamine. In embodiments, the polymerase further includes a mutation at amino acid position 469, wherein the mutation is asparagine, aspartic acid, glutamic acid, or glutamine. In embodiments, the polymerase further includes a mutation at amino acid position 469 or the amino acid position corresponding to position 469, wherein the mutation is asparagine. In embodiments, the polymerase further includes a mutation at amino acid position 469, wherein the mutation is asparagine. In embodiments, the polymerase further includes a mutation at amino acid position 469 or the amino acid position corresponding to position 469, wherein the mutation is aspartic acid. In embodiments, the polymerase further includes a mutation at amino acid position 469, wherein the mutation is aspartic acid. In embodiments, the polymerase further includes a mutation at amino acid position 469 or the amino acid position corresponding to position 469, wherein the mutation is glutamic acid. In embodiments, the polymerase further includes a mutation at amino acid position 469, wherein the mutation is glutamic acid. In embodiments, the polymerase further includes a mutation at amino acid position 469 or the amino acid position corresponding to position 469, wherein the mutation is glutamine. In embodiments, the polymerase further includes a mutation at amino acid position 469, wherein the mutation is glutamine.

In embodiments, the polymerase further includes a mutation at amino acid position 472 or the amino acid position corresponding to position 472, wherein the mutation is asparagine, aspartic acid, glutamic acid, or glutamine. In embodiments, the polymerase further includes a mutation at amino acid position 472 wherein the mutation is asparagine, aspartic acid, glutamic acid, or glutamine. In embodiments, the polymerase further includes a mutation at amino acid position 472 or the amino acid position corresponding to position 472, wherein the mutation is asparagine. In embodiments, the polymerase further includes a mutation at amino acid position 472 wherein the mutation is asparagine. In embodiments, the polymerase further includes a mutation at amino acid position 472 or the amino acid position corresponding to position 472, wherein the mutation is aspartic acid. In embodiments, the polymerase further includes a mutation at amino acid position 472 wherein the mutation is aspartic acid. In embodiments, the polymerase further includes a mutation at amino acid position 472 or the amino acid position corresponding to position 472, wherein the mutation is glutamic acid. In embodiments, the polymerase further includes a mutation at amino acid position 472 wherein the mutation is glutamic acid. In embodiments, the polymerase further includes a mutation at amino acid position 472 or the amino acid position corresponding to position 472, wherein the mutation is glutamine. In embodiments, the polymerase further includes a mutation at amino acid position 472 wherein the mutation is glutamine.

In embodiments, the mutation at amino acid position 472 or the amino acid position corresponding to position 472, wherein the mutation is asparagine or glutamic acid. In embodiments, the mutation at amino acid position 472 is asparagine or glutamic acid. In embodiments, the mutation at amino acid position 472 or the amino acid position corresponding to position 472, wherein the mutation is asparagine. In embodiments, the mutation at amino acid position 472 is asparagine. In embodiments, the mutation at amino acid position 472 or the amino acid position corresponding to position 472, wherein the mutation is glutamic acid. In embodiments, the mutation at amino acid position 472 is glutamic acid.

In embodiments, the polymerase includes a mutation at amino acid position 477 or an amino acid position corresponding to position 477, wherein the mutation is isoleucine, leucine, tryptophan, phenylalanine, alanine, or glycine. In embodiments, the mutation at amino acid position 477 is isoleucine. In embodiments, the mutation at amino acid position 477 is leucine. In embodiments, the mutation at amino acid position 477 is alanine. In embodiments, the mutation at amino acid position 477 is glycine. In embodiments, the mutation at amino acid position 477 is tryptophan. In embodiments, the mutation at amino acid position 477 is phenylalanine.

In embodiments, the polymerase includes a mutation at amino acid position 478 or an amino acid position corresponding to position 478, wherein the mutation is glycine, alanine, valine, leucine, or isoleucine. In embodiments, the polymerase includes a mutation at amino acid position 478 or an amino acid position corresponding to position 478, wherein the mutation is glycine. In embodiments, the polymerase includes a mutation at amino acid position 478 or an amino acid position corresponding to position 478, wherein the mutation is valine. In embodiments, the polymerase includes a mutation at amino acid position 478 or an amino acid position corresponding to position 478, wherein the mutation is leucine. In embodiments, the polymerase includes a mutation at amino acid position 478 or an amino acid position corresponding to position 478, wherein the mutation is isoleucine. In embodiments, the polymerase includes a mutation at amino acid position 478 or an amino acid position corresponding to position 478, wherein the mutation is alanine.

In embodiments, the polymerase includes a mutation at amino acid position 491 or an amino acid position corresponding to position 491, wherein the mutation is valine, leucine, or glycine. In embodiments, the mutation at amino acid position 491 is valine. In embodiments, the mutation at amino acid position 491 is leucine. In embodiments, the mutation at amino acid position 491 is glycine.

In embodiments, the polymerase includes a mutation at amino acid position 520 or an amino acid position corresponding to position 520, wherein the mutation is histidine, lysine, or arginine. In embodiments, the polymerase includes a mutation at amino acid position 520 or an amino acid position corresponding to position 520, wherein the mutation is histidine. In embodiments, the polymerase includes a mutation at amino acid position 520 or an amino acid position corresponding to position 520, wherein the mutation is lysine. In embodiments, the polymerase includes a mutation at amino acid position 520 or an amino acid position corresponding to position 520, wherein the mutation is arginine.

In embodiments, the polymerase includes a mutation at amino acid position 563 or an amino acid position corresponding to amino acid position 563, wherein the mutation is aspartic acid, glycine, asparagine, or glutamine. In embodiments, the mutation at amino acid position 563 is aspartic acid. In embodiments, the mutation at amino acid position 563 is glycine. In embodiments, the mutation at amino acid position 563 is asparagine. In embodiments, the mutation at amino acid position 563 is glutamine.

In embodiments, the mutation at amino acid position 579 or the amino acid position corresponding to position 579 is leucine, isoleucine, valine, alanine, or glycine. In embodiments, the mutation at amino acid position 579 or the amino acid position corresponding to position 579 is leucine, isoleucine, valine, alanine, or glycine. In embodiments, the mutation at amino acid position or the amino acid position corresponding to position 579 is leucine. In embodiments, the mutation at amino acid position is leucine. In embodiments, the mutation at amino acid position or the amino acid position corresponding to position 579 is isoleucine. In embodiments, the mutation at amino acid position is isoleucine. In embodiments, the mutation at amino acid position or the amino acid position corresponding to position 579 is valine. In embodiments, the mutation at amino acid position is valine. In embodiments, the mutation at amino acid position or the amino acid position corresponding to position 579 is alanine. In embodiments, the mutation at amino acid position is alanine. In embodiments, the mutation at amino acid position or the amino acid position corresponding to position 579 is glycine. In embodiments, the mutation at amino acid position 579 is glycine.

In embodiments, the mutation at amino acid position 579 or the amino acid position corresponding to position 579 is glycine, arginine, histidine, or lysine. In embodiments, the mutation at amino acid position or the amino acid position corresponding to position 579 is glycine. In embodiments, the mutation at amino acid position 579 is glycine. In embodiments, the mutation at amino acid position or the amino acid position corresponding to position 579 is arginine. In embodiments, the mutation at amino acid position is arginine.

In embodiments, the mutation at amino acid position or the amino acid position corresponding to position 579 is histidine. In embodiments, the mutation at amino acid position is histidine. In embodiments, the mutation at amino acid position or the amino acid position corresponding to position 579 is lysine. In embodiments, the mutation at amino acid position is lysine. In embodiments, the polymerase includes a glycine at amino acid position 597 and a mutation at amino acid position 588 (e.g., a lysine or valine at amino acid position 588).

In embodiments, the mutation at amino acid position 581 or the amino acid position corresponding to position 581 is leucine, isoleucine, valine, alanine, or glycine. In embodiments, the mutation at amino acid position 581 or the amino acid position corresponding to position 581 is leucine, isoleucine, valine, alanine, or glycine. In embodiments, the mutation at amino acid position or the amino acid position corresponding to position 581 is leucine. In embodiments, the mutation at amino acid position is leucine. In embodiments, the mutation at amino acid position or the amino acid position corresponding to position 581 is isoleucine. In embodiments, the mutation at amino acid position is isoleucine. In embodiments, the mutation at amino acid position or the amino acid position corresponding to position 581 is valine. In embodiments, the mutation at amino acid position is valine. In embodiments, the mutation at amino acid position or the amino acid position corresponding to position 581 is alanine. In embodiments, the mutation at amino acid position is alanine. In embodiments, the mutation at amino acid position or the amino acid position corresponding to position 581 is glycine. In embodiments, the mutation at amino acid position 581 is glycine.

In embodiments, the polymerase includes a mutation at amino acid position 588 or an amino acid position corresponding to position 588; wherein the second mutation is leucine, isoleucine, valine, alanine, or glycine. In embodiments, the mutation at amino acid position 588 or an amino acid position corresponding to position 588 is leucine. In embodiments, the mutation at amino acid position 588 or an amino acid position corresponding to position 588 is isoleucine. In embodiments, the mutation at amino acid position 588 or an amino acid position corresponding to position 588 is valine. In embodiments, the mutation at amino acid position 588 or an amino acid position corresponding to position 588 is alanine. In embodiments, the mutation at amino acid position 588 or an amino acid position corresponding to position 588 is glycine. In embodiments, the mutation at amino acid position 588 or an amino acid position corresponding to position 588 is leucine, isoleucine, valine, or alanine. In embodiments, the mutation at amino acid position 588 or an amino acid position corresponding to position 588 is leucine or valine.

In embodiments, the polymerase includes a mutation at amino acid position 591 or an amino acid position corresponding to position 591, wherein the mutation is isoleucine, leucine, valine, phenylalanine, alanine, or glycine. In embodiments, the mutation at amino acid position 591 is isoleucine. In embodiments, the mutation at amino acid position 591 is leucine. In embodiments, the mutation at amino acid position 591 is alanine. In embodiments, the mutation at amino acid position 591 is glycine. In embodiments, the mutation at amino acid position 591 is valine.

In embodiments, the polymerase includes a mutation at amino acid position 606 or an amino acid position corresponding to position 606, wherein the mutation is isoleucine, leucine, valine, phenylalanine, alanine, or glycine. In embodiments, the mutation at amino acid position 606 is isoleucine. In embodiments, the mutation at amino acid position 606 is leucine. In embodiments, the mutation at amino acid position 606 is alanine. In embodiments, the mutation at amino acid position 606 is glycine. In embodiments, the mutation at amino acid position 606 is valine.

In embodiments, the polymerase includes a mutation at amino acid position 637 or an amino acid position corresponding to amino acid position 637, wherein the mutation is aspartic acid, lysine, glutamic acid, asparagine, or glutamine. In embodiments, the mutation at amino acid position 637 is aspartic acid. In embodiments, the mutation at amino acid position 637 is glutamic acid. In embodiments, the mutation at amino acid position 637 is asparagine. In embodiments, the mutation at amino acid position 637 is glutamine. In embodiments, the mutation at amino acid position 637 is lysine.

In embodiments, the polymerase includes a mutation at amino acid position 634 or an amino acid position corresponding to amino acid position 634, wherein the mutation is tyrosine, phenylalanine, methionine, tryptophan, or leucine. In embodiments, the mutation at amino acid position 634 is tyrosine. In embodiments, the mutation at amino acid position 634 is phenylalanine. In embodiments, the mutation at amino acid position 634 is methionine. In embodiments, the mutation at amino acid position 634 is tryptophan. In embodiments, the mutation at amino acid position 634 is leucine.

In embodiments, the polymerase includes a mutation at amino acid position 635 or an amino acid position corresponding to amino acid position 635, wherein the mutation is aspartic acid, lysine, glutamic acid, asparagine, or glutamine. In embodiments, the mutation at amino acid position 635 is aspartic acid. In embodiments, the mutation at amino acid position 635 is glutamic acid. In embodiments, the mutation at amino acid position 635 is asparagine. In embodiments, the mutation at amino acid position 635 is glutamine. In embodiments, the mutation at amino acid position 635 is lysine.

In embodiments, the polymerase includes a mutation at amino acid position 646 or an amino acid position corresponding to amino acid position 646, wherein the mutation is glutamic acid, glycine, asparagine, or glutamine. In embodiments, the mutation at amino acid position 646 is glutamic acid. In embodiments, the mutation at amino acid position 646 is glycine. In embodiments, the mutation at amino acid position 646 is asparagine. In embodiments, the mutation at amino acid position 646 is glutamine.

In embodiments, the mutation at amino acid position 726 or the amino acid position corresponding to position 726 is aspartic acid, glutamic acid, asparagine, or glutamine. In embodiments, the mutation at amino acid position 726 is aspartic acid, glutamic acid, asparagine, or glutamine. In embodiments, the mutation at amino acid position 726 or the amino acid position corresponding to position 726 is aspartic acid. In embodiments, the mutation at amino acid position 726 is aspartic acid. In embodiments, the mutation at amino acid position 726 or the amino acid position corresponding to position 726 is glutamic acid. In embodiments, the mutation at amino acid position 726 is glutamic acid. In embodiments, the mutation at amino acid position 726 or the amino acid position corresponding to position 726 is asparagine. In embodiments, the mutation at amino acid position 726 is asparagine. In embodiments, the mutation at amino acid position 726 or the amino acid position corresponding to position 726 is glutamine. In embodiments, the mutation at amino acid position 726 is glutamine.

In embodiments, the polymerase includes a mutation at amino acid position 730 or an amino acid position corresponding to amino acid position 730, wherein the mutation is aspartic acid, glutamic acid, glycine, asparagine, or glutamine. In embodiments, the mutation at amino acid position 730 is aspartic acid. In embodiments, the mutation at amino acid position 730 is glutamic acid. In embodiments, the mutation at amino acid position 730 is glycine. In embodiments, the mutation at amino acid position 730 is asparagine. In embodiments, the mutation at amino acid position 730 is glutamine.

In embodiments, the polymerase includes a mutation at amino acid position 742 or an amino acid position corresponding to position 742, wherein the mutation is leucine, isoleucine, alanine, or glycine. In embodiments, the mutation at amino acid position 742 is leucine. In embodiments, the mutation at amino acid position 742 is isoleucine. In embodiments, the mutation at amino acid position 742 is alanine. In embodiments, the mutation at amino acid position 742 is glycine.

In embodiments, the polymerase includes a mutation at amino acid position 766 or an amino acid position corresponding to position 766, wherein the mutation is proline or glycine. In embodiments, the mutation at amino acid position 766 is proline. In embodiments, the mutation at amino acid position 766 is glycine.

In embodiments, the polymerase includes a mutation at amino acid position 769 or an amino acid position corresponding to position 769, wherein the mutation is leucine, isoleucine, arginine, valine, alanine, or glycine. In embodiments, the polymerase includes a mutation at amino acid position 769 or an amino acid position corresponding to position 769, wherein the mutation is leucine. In embodiments, the polymerase includes a mutation at amino acid position 769 or an amino acid position corresponding to position 769, wherein the mutation is isoleucine. In embodiments, the polymerase includes a mutation at amino acid position 769 or an amino acid position corresponding to position 769, wherein the mutation is valine. In embodiments, the polymerase includes a mutation at amino acid position 769 or an amino acid position corresponding to position 769, wherein the mutation is alanine. In embodiments, the polymerase includes a mutation at amino acid position 769 or an amino acid position corresponding to position 769, wherein the mutation is glycine. In embodiments, the polymerase includes a mutation at amino acid position 769 or an amino acid position corresponding to position 769, wherein the mutation is arginine.

In embodiments, the polymerase further includes an amino acid position 520 or the amino acid position corresponding to position 520, wherein the mutation is histidine, lysine, or arginine; a mutation at amino acid position 465 or the amino acid position corresponding to position 465, wherein the mutation is asparagine, aspartic acid, glutamic acid, or glutamine; and/or a mutation at amino acid position 491 or the amino acid position corresponding to position 491, wherein the mutation is glycine, valine, leucine, or isoleucine. In embodiments, the polymerase further includes an amino acid position 520, wherein the mutation is histidine, lysine, or arginine; a mutation at amino acid position 465, wherein the mutation is asparagine, aspartic acid, glutamic acid, or glutamine; and/or a mutation at amino acid position 491, wherein the mutation is glycine, valine, leucine, or isoleucine.

In embodiments, the polymerase further includes a mutation at amino acid position 215 or the amino acid position corresponding to position 215, wherein the mutation is glycine, asparagine, aspartic acid, glutamic acid, or glutamine. In embodiments, the mutation at amino acid position 215 or the amino acid position corresponding to position 215, wherein the mutation is asparagine or glutamic acid. In embodiments, the mutation at amino acid position 215 is asparagine or glutamic acid. In embodiments, the mutation at amino acid position 215 or the amino acid position corresponding to position 215, wherein the mutation is asparagine. In embodiments, the mutation at amino acid position 215 is asparagine. In embodiments, the mutation at amino acid position 215 or the amino acid position corresponding to position 215, wherein the mutation is glutamic acid. In embodiments, the mutation at amino acid position 215 is glutamic acid. In embodiments, the mutation at amino acid position 215 is glycine.

In embodiments, the polymerase further includes a mutation at amino acid position 581 or the amino acid position corresponding to position 581, wherein the mutation is glycine, asparagine, aspartic acid, glutamic acid, or glutamine. In embodiments, the mutation at amino acid position 581 or the amino acid position corresponding to position 581, wherein the mutation is asparagine or glutamic acid. In embodiments, the mutation at amino acid position 581 is asparagine or glutamic acid. In embodiments, the mutation at amino acid position 581 or the amino acid position corresponding to position 581, wherein the mutation is asparagine. In embodiments, the mutation at amino acid position 581 is asparagine. In embodiments, the mutation at amino acid position 581 or the amino acid position corresponding to position 581, wherein the mutation is glutamic acid. In embodiments, the mutation at amino acid position 581 is glutamic acid. In embodiments, the mutation at amino acid position 581 is glycine. In embodiments, the polymerase includes a glycine at amino acid position 581 and a leucine or valine at amino acid position 588. In embodiments, the polymerase includes a glycine at amino acid position 581 and a leucine or valine at amino acid position 588, and a valine, alanine, or leucine at amino acid position 591.

In embodiments, the polymerase further includes a mutation at amino acid position 261 or the amino acid position corresponding to position 261. In embodiments, the polymerase further includes a mutation at amino acid position 261 or the amino acid position corresponding to position 261, wherein the mutation is tyrosine. In embodiments, the polymerase further includes a mutation at amino acid position 26 or the amino acid position corresponding to position 26, wherein the mutation is serine, threonine, asparagine, or glutamine. In embodiments, the polymerase further includes a mutation at amino acid position 245 or the amino acid position corresponding to position 245, wherein the mutation is serine, threonine, asparagine, or glutamine. In embodiments, the polymerase further includes a mutation at amino acid position 634 or the amino acid position corresponding to position 634, wherein the mutation is tyrosine, tryptophan, arginine, or lysine. In embodiments, the polymerase further includes a mutation at amino acid position 726 or the amino acid position corresponding to position 726, wherein the mutation is glycine, asparagine, aspartic acid, glutamic acid, or glutamine. In embodiments, the polymerase further includes a mutation at amino acid position 13 or the amino acid position corresponding to position 13, wherein the mutation is asparagine, aspartic acid, glutamic acid, or glutamine. In embodiments, the polymerase further includes a mutation at amino acid position 23 or the amino acid position corresponding to position 23, wherein the mutation is tyrosine, tryptophan, arginine, or lysine. In embodiments, the polymerase further includes a mutation at amino acid position 520 or the amino acid position corresponding to position 520, wherein the mutation is histidine, arginine, or lysine. In embodiments, the polymerase further includes a mutation at amino acid position 91 or the amino acid position corresponding to position 91, wherein the mutation is histidine, arginine, or lysine. In embodiments, the polymerase further includes a mutation at amino acid position 467 or the amino acid position corresponding to position 467, wherein the mutation is cysteine, serine, or glycine. In embodiments, the polymerase further includes a mutation at amino acid position 469 or the amino acid position corresponding to position 469, wherein the mutation is serine, threonine, or glycine. In embodiments, the polymerase further includes a mutation at amino acid position 742 or the amino acid position corresponding to position 742, wherein the mutation is alanine, leucine, valine, or glycine. In embodiments, the polymerase further includes a mutation at amino acid position 769 or the amino acid position corresponding to position 769, wherein the mutation is arginine, serine, threonine, asparagine, glutamine, alanine, leucine, valine, or glycine.

In embodiments, the mutation at amino acid position 742 or the amino acid position corresponding to position 742 is leucine, isoleucine, alanine, or glycine. In embodiments, the mutation at amino acid position 742 is leucine, isoleucine, alanine, or glycine. In embodiments, the mutation at amino acid or the amino acid position corresponding to position 742 is leucine. In embodiments, the mutation at amino acid position 742 is leucine. In embodiments, the mutation at amino acid or the amino acid position corresponding to position 742 is isoleucine. In embodiments, the mutation at amino acid position 742 is isoleucine. In embodiments, the mutation at amino acid position or the amino acid position corresponding to position 742 is alanine. In embodiments, the mutation at amino acid position 742 is alanine. In embodiments, the mutation at amino acid position or the amino acid position corresponding to position 742 is glycine. In embodiments, the mutation at amino acid position 742 is glycine.

In embodiments, the mutation at amino acid position 742 or the amino acid position corresponding to position 742 is alanine or glycine. In embodiments, the mutation at amino acid position 742 is alanine or glycine. In embodiments, the mutation at amino acid position or the amino acid position corresponding to position 742 is alanine. In embodiments, the mutation at amino acid position 742 is alanine. In embodiments, the mutation at amino acid position or the amino acid position corresponding to position 742 is glycine. In embodiments, the mutation at amino acid position 742 is glycine.

In embodiments, the polymerase further includes a mutation at amino acid position 97 or an amino acid position corresponding to position 97; amino acid position 13 or an amino acid position corresponding to position 13; amino acid position 726 or an amino acid position corresponding to position 726; and/or amino acid position 241 or an amino acid position corresponding to position 241. In embodiments, the polymerase further includes a mutation at amino acid position 97; amino acid position 13; amino acid position 726; and/or amino acid position 241.

In embodiments, the polymerase includes a glutamine, valine, arginine, or alanine at amino acid position 93 or the amino acid position corresponding to position 93. In embodiments, the polymerase includes a glutamine, valine, arginine, or alanine at amino acid position 93. It is known that the presence of uracil in DNA results in a dramatic increase in the binding affinity of archaeal family B DNA polymerases, stalling further polymerase activity (Lasken R S et al. J. Biol. Chem. 1996, 271 (30):17692-6 and Fogg M J et al. Nature Structural Biology. 2002, 9: 922-7). A specific point mutation in the uracil-binding pocket of these polymerases disrupts uracil binding and allows extension in the presence of uracil without compromising polymerase activity (Norholm MH BMC Biotechnology. 2010, 10:21). Provided herein are novel DNA polymerase variants (e.g., V93Q, V93R, V93A) that disrupt the uracil binding pocket. In embodiments, the polymerase includes a V93Q, V93R, or V93A mutation. In embodiments, the polymerase includes a V93Q mutation. In embodiments, the polymerase includes a V931, V93L, V93N, V93D, or V93E mutation. In embodiments, the polymerase includes an amino acid substitution at position 93. In embodiments, the amino acid substitution at position 93 is a glutamine substitution. In embodiments, the amino acid substitution at position 93 is an arginine substitution. In embodiments, the amino acid substitution at position 93 is an alanine substitution. In embodiments, the amino acid substitution at position 93 is a leucine substitution. In embodiments, the amino acid substitution at position 93 is an isoleucine substitution.

In embodiments, the polymerase includes an alanine at amino acid position 141 or the amino acid position corresponding to position 141; and an alanine at amino acid position 143 or the amino acid position corresponding to position 143. In embodiments, the polymerase includes an alanine at amino acid position 141; and an alanine at amino acid position 143.

In embodiments, the polymerase includes an amino acid substitution at position 141. In embodiments, the amino acid substitution at position 141 is an alanine substitution. In embodiments, the amino acid substitution at position 141 is a glycine substitution.

In embodiments, the polymerase includes an amino acid substitution at position 143. In embodiments, the amino acid substitution at position 143 is an alanine substitution. In embodiments, the amino acid substitution at position 143 is a glycine, alanine, threonine, or serine substitution.

In embodiments, the polymerase includes a leucine or isoleucine at amino acid position 588, or the amino acid position corresponding to position 588; a histidine at amino acid position 520, or the amino acid position corresponding to position 520; a glycine at amino acid position 580, or the amino acid position corresponding to position 580; a glutamic acid at amino acid position 465, or the amino acid position corresponding to position 465; a valine at amino acid position 491, or the amino acid position corresponding to position 491; and/or a glutamic acid at amino acid position 472, or the amino acid position corresponding to position 472. In embodiments, the polymerase includes a leucine or isoleucine at amino acid position 588; a histidine at amino acid position 520; a glycine at amino acid position 580; a glutamic acid at amino acid position 465; a valine at amino acid position 491; and/or a glutamic acid at amino acid position 472.

In embodiments, the polymerase includes a histidine at amino acid position 7, or the amino acid position corresponding to position 7; a valine at amino acid position 491, or the amino acid position corresponding to position 491; an arginine at amino acid position 13, or the amino acid position corresponding to position 13; a histidine at amino acid position 526, or the amino acid position corresponding to position 526; a valine at amino acid position 40, or the amino acid position corresponding to position 40; a glycine at amino acid position 579, or the amino acid position corresponding to position 579; a leucine at amino acid position 75, or the amino acid position corresponding to position 75; a leucine at amino acid position 588, or the amino acid position corresponding to position 588; a cysteine or a histidine at amino acid position 97, or the amino acid position corresponding to position 97; an isoleucine at amino acid position 606, or the amino acid position corresponding to position 606; an aspartic acid at amino acid position 149, or the amino acid position corresponding to position 149; an aspartic acid at amino acid position 635, or the amino acid position corresponding to position 635; an arginine at amino acid position 192, or the amino acid position corresponding to position 192; an aspartic acid at amino acid position 637, or the amino acid position corresponding to position 637; a glutamic acid at amino acid position 199, or the amino acid position corresponding to position 199; an aspartic acid at amino acid position 672, or the amino acid position corresponding to position 672; an isoleucine at amino acid position 241, or the amino acid position corresponding to position 241; an aspartic acid at amino acid position 726, or the amino acid position corresponding to position 726; a leucine at amino acid position 275, or the amino acid position corresponding to position 275; an alanine at amino acid position 742, or the amino acid position corresponding to position 742; a serine at amino acid position 316, or the amino acid position corresponding to position 316; a tyrosine at amino acid position 749, or the amino acid position corresponding to position 749; a glutamine at amino acid position 395, or the amino acid position corresponding to position 395; an aspartic acid at amino acid position 765, or the amino acid position corresponding to position 765; a threonine at amino acid position 469, or the amino acid position corresponding to position 469; an arginine at amino acid position 769, or the amino acid position corresponding to position 769; and/or an asparagine at amino acid position 472, or the amino acid position corresponding to position 472. In embodiments, the polymerase includes a histidine at amino acid position 7; a valine at amino acid position 491; an arginine at amino acid position 13; a histidine at amino acid position 526; a valine at amino acid position 40; a glycine at amino acid position 579; a leucine at amino acid position 75; a leucine at amino acid position 588; a cysteine or a histidine at amino acid position 97; an isoleucine at amino acid position 606; an aspartic acid at amino acid position 149; an aspartic acid at amino acid position 635; an arginine at amino acid position 192; an aspartic acid at amino acid position 637; a glutamic acid at amino acid position 199; an aspartic acid at amino acid position 672; an isoleucine at amino acid position 241; an aspartic acid at amino acid position 726; a leucine at amino acid position 275; an alanine at amino acid position 742; a serine at amino acid position 316; a tyrosine at amino acid position 749; a glutamine at amino acid position 395; an aspartic acid at amino acid position 765; a threonine at amino acid position 469; an arginine at amino acid position 769; and/or an asparagine at amino acid position 472.

In embodiments, the polymerase includes an amino acid sequence that is at least 85%, at least 90%, or at least 95% identical to a continuous 500 amino acid sequence within SEQ ID NO: 1. In embodiments, the polymerase includes an amino acid sequence that is at least 80%, at least 85%, at least 90%, or at least 95% identical to a continuous 600 amino acid sequence within SEQ ID NO: 1. In embodiments, the polymerase includes an amino acid sequence that is at least 80%, at least 85%, at least 90%, or at least 95% identical to a continuous 700 amino acid sequence within SEQ ID NO: 1. In embodiments, the polymerase includes an amino acid sequence that is at least 85% identical to a continuous 500 amino acid sequence within SEQ ID NO: 1. In embodiments, the polymerase includes an amino acid sequence that is at least 90% identical to a continuous 500 amino acid sequence within SEQ ID NO: 1. In embodiments, the polymerase includes an amino acid sequence that is at least 95% identical to a continuous 500 amino acid sequence within SEQ ID NO: 1. In embodiments, the polymerase includes an amino acid sequence that is at least 85% identical to a continuous 600 amino acid sequence within SEQ ID NO: 1. In embodiments, the polymerase includes an amino acid sequence that is at least 90% identical to a continuous 600 amino acid sequence within SEQ ID NO: 1. In embodiments, the polymerase includes an amino acid sequence that is at least 95% identical to a continuous 600 amino acid sequence within SEQ ID NO: 1. In embodiments, the polymerase includes an amino acid sequence that is at least 85% identical to a continuous 700 amino acid sequence within SEQ ID NO: 1. In embodiments, the polymerase includes an amino acid sequence that is at least 90% identical to a continuous 700 amino acid sequence within SEQ ID NO: 1. In embodiments, the polymerase includes an amino acid sequence that is at least 95% identical to a continuous 700 amino acid sequence within SEQ ID NO: 1.

In embodiments, the polymerase does not comprise the following mutations: (L4095); (L409Q); (L409Y); or (L409F); (Y410G); (Y410A); or (Y4105); and (P411S); (P411I); (P411C); (P411A). In embodiments, the polymerase does not comprise L4095; Y410G; and P411I. In embodiments, the polymerase does not comprise L4095; Y410A; and P411I. In embodiments, the polymerase does not comprise L4095; Y410G; and P411S. In embodiments, the polymerase does not comprise L4095; Y410A; and P411S. In embodiments, the polymerase is not a wild type enzyme. In embodiments, the polymerase is a synthetic polymerase.

Functionally equivalent, positionally equivalent and homologous amino acids within the wild type amino acid sequences of two different polymerases do not necessarily have to be the same type of amino acid residue, although functionally equivalent, positionally equivalent and homologous amino acids are commonly conserved. By way of example, the motif A region of 9° N polymerase has the sequence LYP, the functionally homologous region of Vent™ polymerase also has sequence LYP. In the case of these two polymerases the homologous amino acid sequences are identical, however homologous regions in other polymerases may have different amino acid sequence. In embodiments, when describing an amino acid functionally equivalent to amino acid position 409, or describing an amino acid position functionally equivalent to amino acid position 409, positional equivalence and/or functional equivalence is referring to amino acid position 409 of SEQ ID NO:1 or an amino acid at a position in a polymerase at least 80% identical to a continuous 500 amino acid sequence within SEQ ID NO: 1 that is equivalent to position 409 of SEQ ID NO:1. A person having ordinary skill in the art would recognize a positional equivalent of amino acid position 409 by performing a sequence alignment given that the polymerase must be at least 80% identical to a continuous 500 amino acid sequence within SEQ ID NO: 1.

Modified nucleotides that contain a unique cleavably-linked fluorophore and a reversible-terminating moiety capping the 3′-OH group, for example, those described in U.S. 2017/0130051, WO 2017/058953, WO 2019/164977, and U.S. Pat. No. 10,738,072, have shown sensitivity to cysteines present in sequencing polymerases. The cysteines normally form a disulfide bridge, however in the presence of sequencing solutions and conditions, the disulfide bridge may break to form two reactive thiols. These thiols may act to prematurely cleave the linker and/or reversible terminator, acting as a weak reducing agent, increasing asynchronous shifts in sequencing runs that are detrimental to sequencing accuracy. There is a need for a sequencing polymerase that has reduced interference with the modified nucleotides used in sequencing applications.

Disulfide bridges are highly conserved among thermophilic polymerases. Structural data has implied that disulfides do not play a direct role in catalysis or substrate binding, but rather, it has been suggested that they contribute to enzyme thermostability. Studies assessing the removal of disulfides from family B archaeal polymerases have shown that the disulfides make a contribution to thermostability (Killelea T. and Connolly BA. ChemBioChem. 2011, 12:1330-36). The applicants discovered the polymerases are capable of incorporating modified nucleotides at high temperatures, and advantageously do not degrade the nucleotides permitting longer sequencing read lengths and better accuracy. Provided herein are novel family B DNA polymerases wherein the conserved cysteines are mutated. In embodiments, the polymerase includes mutations at positions 429, 443, 507, and 510 to serine amino acids. While serine was chosen as an initial mutation, any amino acid that eliminates the ability to form free thiols and does not perturb the stability nor function of the polymerase is envisioned (e.g., glycine, threonine, selenocysteine or alanine). Each of the variants lacking a cysteine were capable of incorporating modified nucleotides, and advantageously, the modified nucleotides exhibited greater stability (i.e., did not prematurely deblock or lose the detectable moiety) relative to a polymerase that contained one or more cysteines.

In embodiments, the polymerase includes an amino acid substitution at position 429. The amino acid substitution at position 429 may be a serine, glycine, threonine, asparagine, or alanine substitution. The amino acid substitution at position 429 may be a serine substitution. In embodiments, the substitution at position 429 includes a polar amino acid (e.g., threonine, asparagine, or glutamine). In embodiments, the amino acid substitution at position 429 is a selenocysteine.

In embodiments, the polymerase includes an amino acid substitution at position 443. The amino acid substitution at position 443 may be a serine, glycine, threonine, asparagine, or alanine substitution. The amino acid substitution at position 443 may be a serine substitution. In embodiments, the substitution at position 443 includes a polar amino acid (e.g., threonine, asparagine, or glutamine). In embodiments, the amino acid substitution at position 443 is a selenocysteine.

In embodiments, the polymerase further includes an amino acid substitution mutation at positions 429 and 443. The amino acid substitutions at positions 429 and 443 may be serine substitutions.

In embodiments, the polymerase includes V93Q, R97H, M129A, D141A, E143A, T144A, L409A, Y410G, A486V, T515S, and A640L. In embodiments, the polymerase includes V93Q, M129A, D141A, E143A, T144A, L409A, Y410G, A486V, T515S, F588L, and A640L. In embodiments, the polymerase includes V93Q, M129A, D141A, E143A, T144A, L409A, Y410G, A486V, T515S, Q520H, E581G, F588V, and A640L. In embodiments, the polymerase includes V93Q, M129A, D141A, E143A, T144A, L409A, Y410G, K465E, A486V, T515S, and A640L. In embodiments, the polymerase includes N23Y, V93Q, M129A, D141A, E143A, T144A, L409A, Y410G, A486V, T515S, E579G, F588L, and A640L. In embodiments, the polymerase includes F75L, V93Q, M129A, D141A, E143A, T144A, L409A, Y410G, A486V, T515S, F588L, and A640L. In embodiments, the polymerase includes V93Q, M129A, D141A, E143A, T144A, L409A, Y410G, A486V, T515S, F588L, T6061, and A640L. In embodiments, the polymerase includes V93Q, M129A, D141A, E143A, T144A, L409A, Y410G, A486V, T515S, R526H, F588L, and A640L. In embodiments, the polymerase includes V93Q, M129A, D141A, E143A, T144A, K199E, L409A, Y410G, A486V, T515S, F588L, and A640L. In embodiments, the polymerase includes V93Q, M129A, D141A, E143A, T144A, L409A, Y410G, A486V, T515S, F588L, A640L, and G765D. In embodiments, the polymerase includes V93Q, M129A, D141A, E143A, T144A, L409A, Y410G, A486V, T515S, Q520H, F588L, and A640L. In embodiments, the polymerase includes V93Q, M129A, D141A, E143A, T144A, T349L, L409A, Y410G, A486V, T515S, and A640L.

In embodiments, the polymerase includes V93Q, R97H, M129A, D141A, E143A, T144A, L409A, Y410G, C429S, C443S, A486V, C507S, C510S, T515S, and A640L. In embodiments, the polymerase includes V93Q, M129A, D141A, E143A, T144A, L409A, Y410G, C429S, C443S, A486V, C507S, C510S, T515S, F588L, and A640L. In embodiments, the polymerase includes V93Q, M129A, D141A, E143A, T144A, L409A, Y410G, C429S, C443S, A486V, C507S, C510S, T515S, Q520H, E581G, F588V, and A640L. In embodiments, the polymerase includes V93Q, M129A, D141A, E143A, T144A, L409A, Y410G, C429S, C443S, K465E, A486V, C507S, C510S, T515S, and A640L. In embodiments, the polymerase includes N23Y, V93Q, M129A, D141A, E143A, T144A, L409A, Y410G, C429S, C443S, A486V, C507S, C510S, T515S, E579G, F588L, and A640L. In embodiments, the polymerase includes F75L, V93Q, M129A, D141A, E143A, T144A, L409A, Y410G, C429S, C443S, A486V, C507S, C510S, T515S, F588L, and A640L. In embodiments, the polymerase includes V93Q, M129A, D141A, E143A, T144A, L409A, Y410G, C429S, C443S, A486V, C507S, C510S, T515S, F588L, T6061, and A640L. In embodiments, the polymerase includes V93Q, M129A, D141A, E143A, T144A, L409A, Y410G, C429S, C443S, A486V, C507S, C510S, T515S, R526H, F588L, and A640L. In embodiments, the polymerase includes V93Q, M129A, D141A, E143A, T144A, K199E, L409A, Y410G, C429S, C443S, A486V, C507S, C510S, T515S, F588L, and A640L. In embodiments, the polymerase includes V93Q, M129A, D141A, E143A, T144A, L409A, Y410G, C429S, C443S, A486V, C507S, C510S, T515S, F588L, A640L, and G765D. In embodiments, the polymerase includes V93Q, M129A, D141A, E143A, T144A, L409A, Y410G, C429S, C443S, A486V, C507S, C510S, T515S, Q520H, F588L, and A640L. In embodiments, the polymerase includes V93Q, M129A, D141A, E143A, T144A, T349L, L409A, Y410G, C429S, C443S, A486V, C507S, C510S, T515S, and A640L.

In embodiments, the polymerase (e.g., a polymerase as described herein) is truncated at the C-terminus. In embodiments, the polymerase (e.g., a polymerase as described herein) is truncated at the C-terminus and retains the ability to incorporate a modified nucleotide. In embodiments, the polymerase (e.g., a polymerase as described herein) is truncated at the C-terminus, wherein the polymerase is truncated to remove at least 20 amino acids from the C-terminus. In embodiments, the polymerase (e.g., a polymerase as described herein) is truncated at the C-terminus, wherein the polymerase is truncated to remove at least 10 amino acids from the C-terminus. In embodiments, the polymerase (e.g., a polymerase as described herein) is truncated at the C-terminus, wherein the polymerase is truncated to remove at least 5 amino acids from the C-terminus. In embodiments, the polymerase (e.g., a polymerase as described herein) is truncated at the C-terminus, wherein the truncation removes 5 to 16 amino acids from the C-terminus. In embodiments, the polymerase (e.g., a polymerase as described herein) is truncated at the C-terminus, wherein the truncation removes 5 amino acids from the C-terminus. In embodiments, the polymerase (e.g., a polymerase as described herein) is truncated at the C-terminus, wherein the truncation removes 10 amino acids from the C-terminus. In embodiments, the polymerase (e.g., a polymerase as described herein) is truncated at the C-terminus, wherein the truncation removes 13 amino acids from the C-terminus. In embodiments, the polymerase (e.g., a polymerase as described herein) is truncated at the C-terminus, wherein the truncation removes 16 amino acids from the C-terminus.

In embodiments, the polymerase includes a polycationic sequence (e.g., a polyhistidine tag, such as a His-6 tag). To facilitate synthesis and/or purification, in embodiments a His6 tag (i.e., six consecutive histidine amino acids) are ligated to the C or N terminus of the polypeptide chain. It is understood that the presence of a His6 tag enables the isolation of peptide or protein products directly from ligation reaction mixtures by Ni-NTA affinity column purification. For example, common polyhistidine tags are formed of six histidine (6xHis tag) residues which are added at the N-terminus preceded by methionine or C-terminus before a stop codon. Alternative polycationic sequences include alternating histidine and glutamine (e.g., three sets of HQ, referred to as an HQ tag) or alternating histidine and asparagine (e.g., six sets of HN, referred to as an HN tag).

In another aspect is provided a nucleic acid encoding a mutant or improved DNA polymerase as described herein, a vector comprising the recombinant nucleic acid, and/or a host cell transformed with the vector. In certain embodiments, the vector is an expression vector. Host cells comprising such expression vectors are useful in methods of the invention for producing the mutant or improved polymerase by culturing the host cells under conditions suitable for expression of the recombinant nucleic acid. The polymerases of the invention may be contained in reaction mixtures and/or kits. The embodiments of the recombinant nucleic acids, host cells, vectors, expression vectors, reaction mixtures and kits are as described herein. The full plasmid nucleic acid sequence used to generate a polymerase is provided in SEQ ID NO: 2.

In an aspect is provided a kit, wherein the kit includes a polymerase as described herein. Generally, the kit includes one or more containers providing a composition, and one or more additional reagents (e.g., a buffer suitable for polynucleotide extension). The kit may also include a template nucleic acid (DNA and/or RNA), one or more primer polynucleotides, nucleotides (including, e.g., deoxyribonucleotides, ribonucleotides, labeled nucleotides, and/or modified nucleotides), buffers, salts, and/or labels (e.g., fluorophores). In embodiments, the kit further includes instructions. In embodiments the kit includes one or more enclosures (e.g., boxes, bottles, or cartridges) containing the relevant reaction reagents and/or supporting materials.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid.

The kit typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

III. Methods

In an aspect, a method of incorporating, and optionally detecting, a modified nucleotide into a nucleic acid sequence is provided. The method includes allowing the following components to interact: (i) a nucleic acid template, (ii) a primer that has an extendible 3′ end, (iii) a nucleotide solution, and (iv) a polymerase (e.g., a DNA polymerase or a thermophilic nucleic acid polymerase as described herein). The polymerase used in the method includes an amino acid sequence that is at least 80% identical to a continuous 500 amino acid sequence within SEQ ID NO: 1 and includes one or more of the mutations described herein. In embodiments, the polymerase includes substitution mutations at positions 141 and 143 of SEQ ID NO: 1. In embodiments, the polymerase further includes at least one amino acid substitution mutation at a position selected from positions 409, 410, and 411 of SEQ ID NO: 1. In embodiments, the polymerase includes a mutation as described herein.

In an aspect is provided a method of incorporating a modified nucleotide into a nucleic acid sequence including combining in a reaction vessel: (i) a nucleic acid template, (ii) a nucleotide solution, and (iii) a polymerase, wherein the polymerase is a polymerase as described herein. In embodiments, the modified nucleotide includes a label (e.g., a label linked to the nucleobase via an optionally cleavable linker). In embodiments, the modified nucleotide includes a reversible terminator moiety (e.g., a polymerase-compatible cleavable moiety bonded to the 3′ oxygen of a nucleotide). In embodiments, the method includes combining the components in a reaction vessel under conditions for incorporating and/or polymerization. Such conditions are known in the art and described herein.

In another aspect is provided a method of sequencing a nucleic acid sequence including: a. hybridizing a nucleic acid template with a primer to form a primer-template hybridization complex; b. contacting the primer-template hybridization complex with a DNA polymerase and modified nucleotides, wherein the DNA polymerase is the polymerase as described herein, wherein the modified nucleotide includes a detectable label; c. subjecting the primer-template hybridization complex to conditions which enable the polymerase to incorporate a modified nucleotide into the primer-template hybridization complex to form a modified primer-template hybridization complex; and d. detecting the detectable label; thereby sequencing a nucleic acid sequence.

In embodiments, the nucleic acid template is DNA, RNA, or analogs thereof. In embodiments, the nucleic acid template includes a primer hybridized to the template. In embodiments, the nucleic acid template is a primer. Primers are usually single-stranded for maximum efficiency in amplification, but may alternatively be double-stranded. If double-stranded, the primer is usually first treated to separate its strands before being used to prepare extension products. This denaturation step is typically affected by heat, but may alternatively be carried out using alkali, followed by neutralization. Thus, a “primer” is complementary to a nucleic acid template, and complexes by hydrogen bonding or hybridization with the template to give a primer/template complex for initiation of synthesis by a polymerase, which is extended by the addition of covalently bonded bases linked at their 3′ end complementary to the template in the process of DNA synthesis. The DNA template for a sequencing reaction will typically comprise a double-stranded region having a free 3′ hydroxyl group which serves as a primer or initiation point for the addition of further nucleotides in the sequencing reaction. The region of the DNA template to be sequenced will overhang this free 3′ hydroxyl group on the complementary strand. The primer bearing the free 3′ hydroxyl group may be added as a separate component (e.g. a short oligonucleotide), which hybridizes to a region of the template to be sequenced. Alternatively, the primer and the template strand to be sequenced may each form part of a partially self-complementary nucleic acid strand capable of forming an intramolecular duplex, such as for example a hairpin loop structure. Nucleotides are added successively to the free 3′ hydroxyl group, resulting in synthesis of a polynucleotide chain in the 5′ to 3′ direction. After each nucleotide addition the nature of the base which has been added will be determined, thus providing sequence information for the DNA template.

In embodiments, the method includes exponential rolling circle amplification (eRCA). Exponential RCA is similar to the linear process except that it uses a second primer having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)).

In embodiments, the method includes hyperbranched rolling circle amplification (HRCA). Hyperbranched RCA uses a second primer complementary to the first amplification product. This allows products to be replicated by a strand-displacement mechanism, which can yield a drastic amplification within an isothermal reaction (Lage et al., Genome Research 13:294-307 (2003), which is incorporated herein by reference in its entirety).

In embodiments, the method includes amplifying a template nucleic acid by extending an amplification primer with a strand-displacing polymerase for about 10 seconds to about 30 minutes. In embodiments, the method includes amplifying a template nucleic acid by extending an amplification primer with a strand-displacing polymerase for about 30 seconds to about 16 minutes. In embodiments, the method includes amplifying a template nucleic acid by extending an amplification primer with a strand-displacing polymerase for about 30 seconds to about 10 minutes. In embodiments, the method includes amplifying a template nucleic acid by extending an amplification primer with a strand-displacing polymerase for about 30 seconds to about 5 minutes. In embodiments, the method includes amplifying a template nucleic acid by extending an amplification primer with a strand-displacing polymerase for about 1 second to about 5 minutes. In embodiments, the method includes amplifying a template nucleic acid by extending an amplification primer with a strand-displacing polymerase for about 1 second to about 2 minutes.

In embodiments, the method includes amplifying a template nucleic acid by extending an amplification primer with a strand-displacing polymerase at a temperature of about 20° C. to about 50° C. In embodiments, the method includes amplifying a template nucleic acid by extending an amplification primer with a strand-displacing polymerase at a temperature of about 30° C. to about 50° C. In embodiments, the method includes amplifying a template nucleic acid by extending an amplification primer with a strand-displacing polymerase at a temperature of about 25° C. to about 45° C. In embodiments, the method includes amplifying a template nucleic acid by extending an amplification primer with a strand-displacing polymerase at a temperature of about 35° C. to about 45° C. In embodiments, the method includes amplifying a template nucleic acid by extending an amplification primer with a strand-displacing polymerase at a temperature of about 35° C. to about 42° C. In embodiments, the method includes amplifying a template nucleic acid by extending an amplification primer with a strand-displacing polymerase at a temperature of about 37° C. to about 40° C.

In embodiments, the nucleotide solution includes modified nucleotides. It is understood that a modified nucleotide and a nucleotide analogue are interchangeable terminology in this context. In embodiments, the nucleotide solution includes labelled nucleotides. In embodiments, the nucleotides include synthetic nucleotides. In embodiments, the nucleotide solution includes modified nucleotides that independently have different reversible terminating moieties (e.g., nucleotide A has an A-term reversible terminator, nucleotide G has an S-term reversible terminator, nucleotide C has an S-term reversible terminator, and nucleotide T has an i-term1 reversible terminator). In embodiments the nucleotide solution contains native nucleotides. In embodiments the nucleotide solution contains labelled nucleotides.

In embodiments, the modified nucleotide has a removable group, for example a label, a blocking group, or protecting group. The removable group includes a chemical group that can be removed from a dNTP analogue such that a DNA polymerase can extend the nucleic acid (e.g., a primer or extension product) by the incorporation of at least one additional nucleotide. In embodiments, the removal group is a reversible terminator.

In embodiments, the modified nucleotide includes a blocking moiety and/or a label moiety. The blocking moiety on a nucleotide can be reversible, whereby the blocking moiety can be removed or modified to allow the 3′ hydroxyl to form a covalent bond with the 5′ phosphate of another nucleotide. The blocking moiety can be effectively irreversible under particular conditions used in a method set forth herein. A label moiety of a nucleotide can be any moiety that allows the nucleotide to be detected, for example, using a spectroscopic method. In embodiments, one or more of the above moieties can be absent from a nucleotide used in the methods and compositions set forth herein. For example, a nucleotide can lack a label moiety or a blocking moiety or both.

In embodiments, the blocking moiety can be located, for example, at the 3′ position of the nucleotide and may be a chemically cleavable moiety such as an allyl group, an azidomethyl group or a methoxymethyl group, or may be an enzymatically cleavable group such as a phosphate. Suitable nucleotide blocking moieties are described in applications WO 2004/018497, U.S. Pat. Nos. 10,738,072, 7,057,026, 7,541,444, WO 96/07669, U.S. Pat. Nos. 5,763,594, 5,808,045, 5,872,244 and 6,232,465, the contents of which are incorporated herein by reference in their entirety. The nucleotides may be labelled or unlabeled. In embodiments, the modified nucleotides with reversible terminators useful in methods provided herein may be 3′-O-blocked reversible or 3′-unblocked reversible terminators. The 3′-O-blocked reversible terminators are known in the art, and may be, for instance, a 3′-ONH₂ reversible terminator, a 3′-O-allyl reversible terminator, or a 3′-O-azidomethyl reversible terminator.

In embodiments, the modified nucleotides useful in methods provided herein can include 3′-unblocked reversible terminators. The 3′-unblocked reversible terminators are known in the art and include for example, the “virtual terminator” as described in U.S. Pat. No. 8,114,973 and the “lightening terminator” as described in U.S. Pat. No. 10,041,115, the contents of which are incorporated herein by reference in their entirety.

In embodiments, the modified nucleotide (also referred to herein as a nucleotide analogue) has the formula:

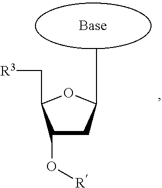

wherein Base is an optionally substituted nucleobase as described herein, R³ is —OH, monophosphate, or polyphosphate or a nucleic acid, and R′ is a reversible terminator. In embodiments, R′ has the formula:

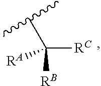

wherein R^{A} and R^{B} are hydrogen or alkyl and R^{C} is the remainder of the reversible terminator (e.g., an azido or SS-C1-C6 alkyl). In embodiments, the nucleotide is

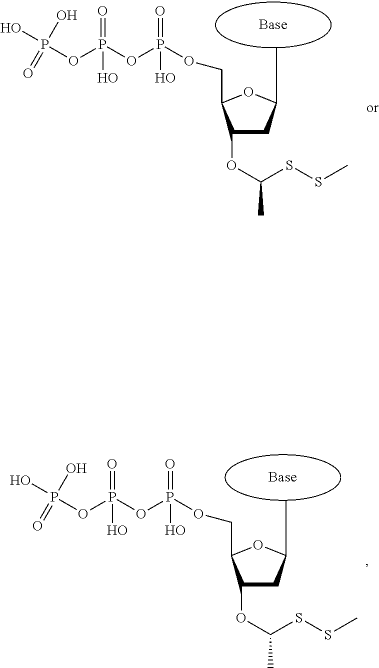 [SMILES: CSS[C@@H](C)O[C@@H]1C[C@H](C)O[C@@H]1COP(=O)(O)OP(=O)(O)OP(=O)(O)O ; CSS[C@H](C)O[C@@H]1C[C@H](C)O[C@@H]1COP(=O)(O)OP(=O)(O)OP(=O)(O)O]

wherein the Base is cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), guanosine or a derivative thereof (e.g., 7-methylguanosine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the base is thymine, cytosine, uracil, adenine, guanine, hypoxanthine, xanthine, theobromine, caffeine, uric acid, or isoguanine.

In embodiments, mutations may include substitution of the amino acid in the parent amino acid sequences with an amino acid, which is not the parent amino acid. In embodiments, the mutations may result in conservative amino acid changes. In embodiments, non-polar amino acids may be converted into polar amino acids (threonine, asparagine, glutamine, cysteine, tyrosine, aspartic acid, glutamic acid or histidine) or the parent amino acid may be changed to an alanine.

In embodiments, the method includes maintaining the temperature at about 55° C. In embodiments, the method includes maintaining the temperature at about 55° C. to about 80° C. In embodiments, the method includes maintaining the temperature at about 60° C. to about 70° C. In embodiments, the method includes maintaining the temperature at about 65° C. to about 75° C. In embodiments, the method includes maintaining the temperature at about 65° C. In embodiments, the method includes maintaining the temperature at about 60° C. In embodiments, the method includes maintaining the temperature at a pH of 8.0 to 11.0. In embodiments, the pH is 9.0 to 11.0. In embodiments, the pH is 9.5. In embodiments, the pH is 10.0. In embodiments, the pH is 8.0, 8.5, 9.0, 9.5, 10.0, 10.5, or 11.0. In embodiments, the pH is from 9.0 to 11.0, and the temperature is about 60° C. to about 70° C. In embodiments, the pH is from 8.5 to 9.5, and the temperature is about 58° C. to about 62° C.

In embodiments, the polymerases described herein have improved polymerase activity (i.e., improved relative to a control). Polymerase activity, in some instances, includes the measurable quantity k_{cat}, k_{cat}/Kₘ, or yields of incorporated nucleotides for a given time period. In embodiments, the polymerases described herein have increased extension activity (i.e., increased relative to a control). Increased extension activity variously refers to an increase in reaction kinetics (increased k_{cat}), increased K_{D}, decreased Kₘ, increased k_{cat}/Kₘ ratio, faster turnover rate, higher turnover number, or other metric that is beneficial to the use of the polypeptide for nucleic acid extension with nucleotides. The polypeptides described herein often incorporate at least 30% more nucleotides than the wild-type polymerase in total or in a given duration of time.

In embodiments, the polymerases described herein often incorporate at least 10%, 20%, 30%, 50%, 75%, 100%, 125%, 150%, 200%, 500%, more nucleotides than a control (e.g., the wild-type polymerase) for a fixed amount of time and same nucleotide concentration. In embodiments, the polymerases described herein incorporate nucleotides at least 1.5, 2, 2.5, 5, 10, 15, 20, 25, or at least 50 times faster than a control (e.g., the wild-type polymerase) for a fixed amount of time. Such measurements are often measured under conditions such as a set period of time, such as at least, at most, or exactly 1, 2, 3, 5, 8, 10, 15, 20, or more than 20 minutes. Such measurements are often measured under conditions such as a set nucleotide concentration, such as less than 10 uM, 10 uM, 20 uM, 50 uM, 100 uM, 200 uM, 300 uM, 500 uM, or more than 500 uM, or any concentration within the range identified herein.

In an aspect is provided a method of sequencing a circular polynucleotide. In embodiments, the method includes circularizing a linear nucleic acid molecule to form a circular polynucleotide. In embodiments, the circularizing includes intramolecular joining of the 5′ and 3′ ends of a linear nucleic acid molecule. In embodiments, the circularizing includes a ligation reaction. In embodiments, the two ends of the linear nucleic acid molecule are ligated directly together. In embodiments, the two ends of the linear nucleic acid molecule are ligated together with the aid of a bridging oligonucleotide (sometimes referred to as a splint oligonucleotide) that is complementary with the two ends of the linear nucleic acid molecule. Methods for forming circular DNA templates are known in the art, for example, linear polynucleotides are circularized in a non-template driven reaction with circularizing ligase, such as CircLigase™, CircLigase™ II, Taq DNA Ligase, HiFi Taq DNA Ligase, T4 DNA ligase, or Ampligase® DNA Ligase. In some embodiments, circularization is facilitated by denaturing double-stranded linear nucleic acids prior to circularization. Residual linear DNA molecules may be optionally digested. In some embodiments, circularization is facilitated by chemical ligation (e.g., click chemistry, e.g., a copper-catalyzed reaction of an alkyne (e.g., a 3′ alkyne) and an azide (e.g., a 5′ azide)). In embodiments, prior to circularization, the linear DNA fragments are A-tailed (e.g., A-tailed using Taq DNA polymerase).

In embodiments, circularization of the linear nucleic acid molecule is performed with CircLigase™ enzyme. In embodiments, circularization of the linear nucleic acid molecule is performed with a thermostable RNA ligase, or mutant thereof. In embodiments, circularization of the linear nucleic acid molecule is performed with an RNA ligase enzyme from bacteriophage TS2126, or mutant thereof. For example, the RNA ligase may be TS2126 RNA ligase, as described in U.S. Pat. Pub. 2005/0266439, which is incorporated herein by reference in its entirety.

In embodiments, circularizing includes ligating a first hairpin and a second hairpin adapter to a linear nucleic acid molecule, thereby forming a circular polynucleotide.

In embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75-500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, the second adapter includes a sample barcode sequence, a molecular identifier sequence, or both a sample barcode sequence and a molecular identifier sequence. In some embodiments, the second adapter includes a sample barcode sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15-250 nucleotides, 15 to 200 nucleotides, 15 to 150 nucleotides, 20 to 100 nucleotides or 20 to 50 nucleotides.

In some embodiments, the loop of a hairpin adapter includes one or more of a primer binding site, a capture nucleic acid binding site (e.g., a nucleic acid sequence complementary to a capture nucleic acid), a UMI, a sample barcode, a sequencing adapter, a label, the like or combinations thereof. In certain embodiments, a loop of a hairpin adapter includes a primer binding site. In certain embodiments, a loop of a hairpin adapter includes a primer binding site and a UMI. In certain embodiments, a loop of a hairpin adapter includes a binding motif.

In some embodiments, the loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogues and/or modified nucleotides bonds.

In some embodiments, the loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loop has a GC content of about or more than about 40%. In embodiments, the loop has a GC content of about or more than about 50%. In embodiments, the loop has a GC content of about or more than about 60%. Non-base modifiers can also be incorporated into a loop of a hairpin adapter to increase Tm, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15-250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In embodiments, circularization includes contacting a double-stranded polynucleotide with at least one protelomerase enzyme. The embodiments, the double-stranded polynucleotide includes complementary protelomerase target sequences at both ends (e.g., the 5′ and 3′ end of each strand includes a protelomerase recognition sequence, or complement thereof). For example, both ends of the target double-stranded DNA molecule are inserted with the double-stranded enzyme recognition DNA molecule (e.g., the double-stranded protelomerase recognition sequence, for example a TeIN protelomerase recognition sequence, has been ligated to each end of the dsDNA molecule). Then, for example, the Escherichia coli phage N15 protelomerase (TelN) catalyzes the double-stranded enzyme recognition DNA molecule on both ends of the target double-stranded DNA molecule to produce a circularized DNA molecule with the target double-stranded DNA molecule circularized.

In embodiments, prior to circularizing one or more linear nucleic acid molecules, the polynucleotide is fragmented to an average length of approximately 150, approximately 250, or approximately 350 base pairs. Fragmentation may be accomplished via methods known in the art (e.g., enzymatic fragmentation, acoustic fragmentation). In embodiments, the polynucleotide is fragmented to generate linear nucleic acid molecules using enzymatic fragmentation or acoustic fragmentation. In embodiments, the input polynucleotide is derived from a fresh or fresh frozen sample and is minimally degraded prior to fragmentation. Next, ssDNA fragments are circularized via CircLigase™ or a method described herein. In some embodiments, circularization is facilitated by denaturing nucleic acids prior to circularization. Residual linear DNA molecules may be optionally digested. This may be accomplished via methods known in the art (e.g., treating with Exo I and/or Exo III enzymes).

In one embodiment, an enzyme is used to ligate the two ends of the linear nucleic acid molecule. For example, linear polynucleotides are circularized in a non-template driven reaction with a circularizing ligase, such as CircLigase™ enzyme, Taq DNA Ligase, HiFi Taq DNA Ligase, T4 DNA ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof. In embodiments, the two ends of the template polynucleotide are ligated together with the aid of a splint primer that is complementary with the two ends of the template polynucleotide. For example, a T4 DNA ligase reaction may be carried out by combining a linear polynucleotide, ligation buffer, ATP, T4 DNA ligase, water, and incubating the mixture at between about 20° C. to about 45° C., for between about 5 minutes to about 30 minutes. In some embodiments, the T4 ligation reaction is incubated at 37° C. for 30 minutes. In some embodiments, the T4 ligation reaction is incubated at 45° C. for 30 minutes. In embodiments, the ligase reaction is stopped by adding Tris buffer with high EDTA and incubating for 1 minute.

In embodiments, a linear nucleic acid molecule may undergo intramolecular circularization (via ligation or annealing) without joining to a circularization adapter (e.g., self-circularization). Circularization (without a circularization adaptor) can be achieved with a ligase at about 4°−35° C. In embodiments, a linear nucleic acid molecule interest can be joined to a loxP adapter and circularization can be mediated by a Cre recombinase enzyme reaction at about 4°−35° C., see for example U.S. Pat. No. 6,465,254, which is incorporated herein by reference.

In embodiments, the circular polynucleotide that is about 100 to about 1000 nucleotides in length, about 100 to about 300 nucleotides in length, about 300 to about 500 nucleotides in length, or about 500 to about 1000 nucleotides in length. In embodiments, the circular polynucleotide is about 300 to about 600 nucleotides in length. In embodiments, the circular polynucleotide is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides in length. In embodiments, the circular polynucleotide molecule is about 100-1000 nucleotides in length. In embodiments, the circular polynucleotide molecule is about 100-300 nucleotides in length. In embodiments, the circular polynucleotide molecule is about 300-500 nucleotides in length. In embodiments, the circular polynucleotide molecule is about 500-1000 nucleotides in length. In embodiments, the circular polynucleotide molecule is about 100 nucleotides. In embodiments, the circular polynucleotide molecule is about 300 nucleotides. In embodiments, the circular polynucleotide molecule is about 500 nucleotides. In embodiments, the circular polynucleotide molecule is about 1000 nucleotides. Circular polynucleotides may be conveniently isolated by a conventional purification column, digestion of non-circular DNA by one or more appropriate exonucleases, or both.

In embodiments, the sequencing includes sequencing by synthesis, sequencing-by-binding, sequencing by hybridization, sequencing by ligation, or pyrosequencing. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444, 7,057,026, and 10,738,072. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Sequencing can be carried out using any suitable sequencing-by-synthesis (SBS) technique, wherein modified nucleotides are added successively to a free 3′ hydroxyl group, typically initially provided by a sequencing primer, resulting in synthesis of a polynucleotide chain in the 5′ to 3′ direction. In embodiments, sequencing includes detecting a sequence of signals. In embodiments, sequencing includes extension of a sequencing primer with labeled nucleotides. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

In embodiments, generating a first sequencing read or a second sequencing read includes sequencing-by-binding (see, e.g., U.S. Pat. Pubs. US2017/0022553 and US2019/0048404, each of which is incorporated herein by reference in its entirety). As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, the sequencing includes a plurality of sequencing cycles. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove label(s) from each incorporated base. Reagents, enzymes and other substances can be removed between steps by washing. Cycles may include repeating these steps, and the sequence of each cluster is read over the multiple repetitions. In embodiments, the sequencing yields reads of greater than 25 bp read length. In embodiments, the sequencing yields reads of greater than 50 bp read length. In embodiments, the sequencing yields reads of greater than 75 bp read length. In embodiments, the sequencing yields reads of greater than 100 bp read length. In embodiments, the sequencing yields reads of greater than 150 bp read length. In embodiments, generating a sequencing read includes determining the identity of the nucleotides in the template polynucleotide.

In embodiments, the sequencing method relies on the use of modified nucleotides that can act as reversible terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide (e.g., a modified, labeled nucleotide). In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof, and (d) a guanine nucleotide, or analog thereof.

In embodiments, the sequencing includes extending a sequencing primer by incorporating a labeled nucleotide, or labeled nucleotide analogue, and detecting the label to generate a signal for each incorporated nucleotide or nucleotide analogue.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1. Focused Mutational Analysis

DNA amplification has many applications in molecular biology research and medical diagnostics. There are two main strategies for amplifying a defined sequence of nucleic acid: polymerase chain reaction (PCR) and isothermal amplification. The polymerase chain reaction relies upon thermal cycling to denature dsDNA templates, followed by annealing primers at specific sites in the denatured template, and extension of the primers by a thermostable DNA polymerase. Isothermal amplification of DNA, as the name implies, typically includes amplification of the dsDNA at a defined temperature. The lack of thermal cycling in isothermal amplification technologies reduces equipment needs and improves the time to answer, especially for point-of-care applications.

A variety of isothermal amplification methods have been developed, for example, strand displacement amplification (SDA) (Walker, G. T. et al. Nucleic Acids Res 20, 1691-6 (1992); and Walker, G. T., Little, M. C, Nadeau, J. G. & Shank, D. D. PNAS 89, 392-6 (1992)), rolling circle amplification (RCA) (Fire, A. & Xu, S. Q. PNAS 92, 4641-5 (1995)), cross priming amplification (CPA) (Xu, G. et al. Sci. Rep. 2, 246; (2012)) and loop mediated amplification (LAMP) (Notomi, T. et al. Nucleic Acids Res 28, e63 (2000)). While some isothermal amplification mechanisms depend upon multiple enzymes, e.g., nickases, recombinases, and ligases, to achieve continuous replication, RCA and LAMP require only polymerases and primers. These methods, like many other isothermal amplification methods, require the use of a DNA polymerase with a strong strand displacement activity to displace downstream DNA, thereby enabling continuous replication without thermal cycling. Additionally, efficient amplification requires elevated temperatures (e.g., 60° C. or greater) to enable the annealing of primers at specific locations on the dsDNA. Thus, a DNA polymerase suitable for these methods must be a thermostable DNA polymerase with a strong strand displacement activity.

Few thermostable, strand displacing enzymes exist. For example, SD DNA polymerase (a mutant Taq DNA polymerase) and the large fragment of Bst DNA polymerase possess favorable characteristics for isothermal amplification. Bacillus stearothermophilus (Bst) DNA Polymerase I is a member of polymerase family A and is one of the most popular enzymes with strand displacement activity because its optimum is about 63° C. Unfortunately, Bst polymerase is limited to temperatures up to 68-70° C.; at temperature 68° C. or higher it is inactivated (Xu, G. et al. Sci. Rep. 2, 246; (2012)). This complicates the workflow, since many isothermal amplification approaches depend on an initial heating step (e.g., to about 95° C.) to denature dsDNA templates.

Despite ongoing research, current modifications to the DNA polymerase still do not show sufficiently high incorporation rates of modified nucleotides. In nucleic acid sequencing applications, the modified nucleotide typically has a reversible terminator at the 3′ position and a modified base (e.g., a base linked to a fluorophore via a cleavable linker). In the case of cleavable linkers attached to the base, there is usually a residual spacer arm left after the cleavage. This residual modification may interfere with incorporation of subsequent nucleotides by polymerase. Therefore, it is highly desirable to have polymerases for carrying out sequencing by synthesis process (SBS) that are tolerable of these scars. In addition to rapid incorporation, the enzyme needs to be stable and have high incorporation fidelity. Balancing incorporation kinetics and fidelity is a challenge. If the mutations in the polymerase result in a rapid average incorporation half-time but are too promiscuous such that the inappropriate nucleotide is incorporated into the primer, this will result in a large source of error in sequencing applications. It is also desirable to design a polymerase capable to incorporating a variety of reversible terminators. Discovering a polymerase that has suitable kinetics and low misincorporation error remains a challenge. In addition to nucleic acid amplification, polymerase mediated nucleic acid sequencing methods (e.g., sequencing-by-synthesis) benefit by using a thermostable, strand-displacing polymerase. For example, it is not uncommon for the template DNA molecules to form secondary structures. This is common in polynucleotides containing elevated amounts of GC nucleotides relative to AT nucleotides. It is widely known that sequencing GC-rich DNA is challenging owing to its inefficient amplification by PCR (Aird D, et al. Genome Biol. 2011; 12(2):R18; and Jakobsen T H, Hansen M A, Jensen P O, et al. PLoS One. 2013;8(7):e68484) and sequencing. Commercially available DNA polymerases capable of incorporating modified nucleotides typically have no strand-displacing activity and are unable able to sequence through this double-stranded formation. Moreover, there are applications involving hairpins or self-priming oligos that require strand-displacing activity, necessitating a first enzyme for strand-displacement activity and a second enzyme for sequencing. Chemical additives may be used, for example betaine, to keep a GC-rich template single-stranded, but it may also cause premature dissociation of the newly synthesized strand from an AT-rich template. It is therefore significantly advantageous to use one single enzyme with the capacity to sequence and possess strand-displacing activity.

An aim of the general experimental plan was to produce a robust, optimized polymerase for nucleic acid sequencing methods. DNA polymerases of the Pyrococcus genus share similar anerobic features as other thermophilic genera (e.g., Archaeoglobus, Thermoautotrophican, Methanococcus), however, Pyrococcus species thrive in higher temperatures, ca 100° C., and tolerate extreme pressures. For example, the area around undersea hot vents, where P. abyssi has been found, there is no sunlight, the temperature is around 98° C.-100° C. and the pressure is about 200 atm. These Pyrococcus polymerases possess inherent properties that are beneficial for sequencing applications.

Directed evolution of enzymes is a process that mimics natural selection in vitro. Compartmentalized self-replication (CSR) is a method of directed evolution where a library containing mutated variants of the enzyme of interest goes through rounds of selective pressure, and over time, the most active or best performing variants are enriched in the library, compared to less active variants, as described in Abil, Z., & Ellington, A. D. (2018). Current Protocols in Chemical Biology, 10, 1-17. During CSR, the enzyme variants and its own encoding genes are compartmentalized in oil emulsions, together with dNTPs and primers. During the emulsion PCR, each enzyme that can surpass the selective pressure is able to replicate its own encoding gene and pass to the next round of selection. Over time, the best performers are enriched in the library.

DNA polymerases carry out crucial functions in many DNA metabolic processes, and due to their ability to catalyze the replication of DNA by incorporating nucleotides into the 3′ end of a primer annealed to a template, DNA polymerases are frequently used in genomic research (e.g., next-generation sequencing, or NGS, technologies). The human genome encodes at least 14 DNA-dependent DNA polymerases, each serving a particular function. The general classification includes five different classes according to their function: DNA polymerase (Pol α) catalyzes DNA replication at Okazaki fragments on the lagging strand; Pol β participates in base-excision repair; Pol γ is involved in mitochondrial DNA synthetic processes; Pol δ participates in lagging-strand synthesis; and Pol c catalyzes the synthesis of the leading strand of chromosomal DNA.

In the context of nucleic acid sequencing, the use of nucleotides bearing a 3′ reversible terminator allows successive nucleotides to be incorporated into a polynucleotide chain in a controlled manner. The DNA template for a sequencing reaction will typically comprise a double-stranded region having a free 3′ hydroxyl group which serves as a primer or initiation point for the addition of further nucleotides in the sequencing reaction. The region of the DNA template to be sequenced will overhang this free 3′ hydroxyl group on the complementary strand. The primer bearing the free 3′ hydroxyl group may be added as a separate component (e.g., a short oligonucleotide) which hybridizes to a region of the template to be sequenced. Following the addition of a single nucleotide to the DNA template, the presence of the 3′ reversible terminator prevents incorporation of a further nucleotide into the polynucleotide chain. While the addition of subsequent nucleotides is prevented, the identity of the incorporated is detected (e.g., exciting a unique detectable label that is linked to the incorporated nucleotide). The reversible terminator is then removed, leaving a free 3′ hydroxyl group for addition of the next nucleotide. The sequencing cycle can then continue with the incorporation of the next blocked, labelled nucleotide.

Sequencing by synthesis of nucleic acids ideally requires the controlled (i.e., one at a time), yet rapid, incorporation of the correct complementary nucleotide opposite the oligonucleotide being sequenced. This allows for accurate sequencing by adding nucleotides in multiple cycles as each nucleotide residue is sequenced one at a time, thus preventing an uncontrolled series of incorporations occurring.

As described herein wild-type Pyrococcus enzymes (e.g., P. horikoshii and P. abyssi) have difficulty incorporating modified nucleotides (e.g., nucleotides including a reversible terminator and/or a cleavable linked base). Relative to a non-modified nucleotide, an incoming modified nucleotide bearing a 3′ reversible terminator increases the activation energy required to orient the phosphate for phosphoryl transfer. To efficiently incorporate modified nucleotides, the DNA polymerase active site needs to be engineered to accommodate a variety of nucleotide structural variants. DNA polymerases evolved mechanisms to ensure selection of the correct nucleotide in order to maintain the integrity and fidelity of the nucleic acid sequence. One such mechanism is the highly conserved region in family B DNA polymerases active site, which includes the amino acids LYP at positions 408-410 of 9° N polymerases. The modifications at amino acid positions D141 and E143 (relative to wild-type) are known to affect exonuclease activity (designated exo-) (see, for example, U.S. Pat. No. 5,756,334 and Southworth et al, 1996 Proc. Natl Acad. Sci USA 93:5281). This 3′-5′ exonuclease activity is absent in some DNA polymerases (e.g., Taq DNA). It is typically beneficial to remove this exonuclease proof-reading activity when using modified nucleotides to prevent the exonuclease removing the unnatural nucleotide after incorporation.

Additional mutations to wild type DNA polymerase enzymes are useful for DNA sequencing applications involving 3′ modified nucleotides. Such changes have previously been made for the Vent and Deep Vent DNA polymerases. As described in WO 2005/024010, modifications to the so-called motif A region, amino acid positions 408-410 of 9° N polymerases, exhibit improved incorporation of nucleotide analogues bearing substituents at the 3′ position of the sugar. Of note, amino acids at positions 408, 409, 410 in a 9° N polymerase are functionally equivalent to amino acids at positions 409, 410, and 411 in wild type P. abyssi and P. horikoshii. This trio of amino acids are in close proximity to the nucleotide that is being incorporated and is strictly conserved across the different types of Family B polymerases; see for example US 2017/0298327 A1; Gueguen, Y., et al (2001), European Journal of Biochemistry, 268: 5961-5969; and Bergen, K., et al. (2013), ChemBioChem, 14: 1058-1062, which are incorporated herein in its entirety for all purposes. Because these three amino acids are in close proximity to the nucleotide being incorporated, a change in the sequence or structure of this motif alters the incorporation kinetics. The amino acids at positions 408, 409, 410 in a 9° N polymerase and Vent™ polymerase are positionally equivalent (i.e., the amino acids at positions 408, 409, 410 in a 9° N polymerase correspond) to amino acids 409,410, and 411 in wild type P. abyssi, and play an important role in incorporating a modified nucleotide into a primer.

Structural analyses of DNA polymerases portray the enzyme as analogous to a human right hand, with three domains: a ‘fingers’ domain that interacts with the incoming dNTP and paired template base, and that closes at each nucleotide addition step; a ‘palm’ domain that catalyzes the phosphoryl-transfer reaction; and a ‘thumb’ domain that interacts with duplex DNA. The finger and palm subdomains of DNA polymerases (e.g., amino acids positions 448-603 of SEQ ID NO:1) are in close proximity to the nucleotide incorporation region. We initially limited our mutational analysis to mutations within the finger and palm subdomains (i.e., examining mutations in amino acids positions 448-603 of SEQ ID NO:1) before broadening out mutational analysis to the entire enzyme sequence (see, Example 2).

For brevity, amino acid mutation nomenclature is used throughout this application. One having skill in the art would understand the amino acid mutation nomenclature, such that D141A refers to aspartic acid (single letter code is D), at position 141, is replaced with alanine (single letter code A). Likewise, it is understood that when an amino acid mutation nomenclature is used and the terminal amino acid code is missing, e.g., P411, it is understood that no mutation was made relative to the wild type. Additionally, for amino acid positions that are frequently mutated herein the wild type amino acid may be recited to emphasize that it is not mutated, for example P411P.

Prior to performing CSR, an error prone PCR library was generated with a target error rate of 6 to 8 mutations per clone using a mutant sequencing enzyme having the sequence SEQ ID NO:2. The mutations were restricted to the approximately 155 amino acids within the finger and palm subdomain, which corresponds to about 465 nucleotides in the gene vector. The library was cloned using a commercial vector pET21b+ via Gibson Assembly, using the Gibson Assembly® Master Mix (NEB Catalog Number E2611S), using the standard protocol described in Gibson, D. G. et. al. (2009) Nature Methods, 343-345 and Gibson, D. G. et al. (2010) Nature Methods, 901-903. Gibson Assembly was developed by Dr. Daniel Gibson and colleagues at the J. Craig Venter Institute and licensed to NEB by Synthetic Genomics, Inc. It allows for successful assembly of multiple DNA fragments, regardless of fragment length or end compatibility. Gibson Assembly efficiently joins multiple overlapping DNA fragments in a single-tube isothermal reaction. The end result is a double-stranded fully sealed DNA molecule that can serve as template for PCR, RCA or a variety of other molecular biology applications. The assembled plasmids containing the diverse inserts were then purified utilizing the Monarch® PCR & DNA Cleanup Kit (NEB Catalog Number T1030L), and then transformed into T7 Express Electrocompetent E. coli cells (NEB Catalog Number C3026J). After plating the transformed cells, the library size as determined by the colony forming units (CFU) was estimated to be about 3.8×10⁶.

The transformed cells were cultured in LB media overnight, and on the next day, protein expression was induced with Isopropyl-beta-D-thiogalactoside (IPDG). On the following day, the emulsion PCR was performed using the oil/surfactant mixtures containing Mineral oil, non-ionic emulsifier (e.g., polaxamer 124, polaxamer 181, or ABIL® EM 90), and a surfactant (e.g., Triton X-100). This mixture was added to the ePCR master mixes, containing a buffer, CSR primer oligonucleotides, BSA, dNTPs, and 1×10⁸ cells. The emulsion was made using a TissueLyser (Qiagen®).

The emulsion PCR program was designed according to the selective pressure used, modulating the number and type of primers for each round. The product from the emulsion PCR was extracted from emulsion using the method known in the art, for example described by Williams et al. Nat Methods. 2006 July;3(7):545-50. A recovery PCR reaction is performed using the product from the ePCR as a template. New primers and PCR programs are designed for this purpose. After that, a third PCR reaction is performed using the Recovery PCR's product as a template. The product from the Re-Amp PCR is then cloned into a commercial vector (pET21b+) via Gibson Assembly, and an additional selection round occurred.

To promote strand displacement, the selective pressure applied included i) progressively increasing the amount of double stranded regions within a nucleic acid template (i.e., increasing the amount of blocking oligos), and ii) decreasing the temperature to promote dsDNA formation. CSR primers would anneal to the template and after a few base pairs, the enzyme encountered one or more blocking primer(s), i.e., an oligonucleotide complementary to a region downstream of the CSR primers. Only the enzyme capable of displacing the double-stranded region would be able to replicate its own encoding gene, and therefore would be enriched in the library. A total of 6 rounds of selective pressure were performed, as described in Table 1 below.

TABLE 1
Selective pressure on the finger and palm subdomains. The conditions
were progressively modulated to decrease the reaction temperature and
progressively increase the amount of double stranded regions within
a nucleic acid template (i.e., the quantity of blocking oligos).

| Round | |
|---|---|
| Number | Selective Pressure |
| Round 1 | Amplification at 72° C. |
| Round 2 | Amplification at 68° C. |
| Round 3 | Amplification at 66° C. and 2× concentration of |
| | blocking oligos |
| Round 4 | Amplification at 63° C. and 2× concentration blocking oligos |
| Round 5 | Amplification at 60° C. and 2× concentration of |
| | blocking oligos |
| Round 6 | Amplification at 60° C. and 4× concentration of |
| | blocking oligos |

The primers were designed to have a sequence that is the complement of a region of template/target DNA to which the primer hybridizes. A number of primer design tools exist, for example PrimerSelect (Plasterer TN. PRIMERSELECT. Primer and probe design, Methods Mol. Biol., 1997, vol. 70 (pg. 291-302)), Primer Express (Applied BiosystemsPrimer Express® Software Version 3.0 Getting Started Guide, 2004), OLIGO 7 (Rychlik W. OLIGO 7 primer analysis software, Methods Mol. Biol., 2007, vol. 402 (pg. 35-60)) and Primer3 (Untergasser, A. et al. Primer3-new capabilities and interfaces. Nucleic Acids Res 40, el15 (2012)). CSR primers contain a non-complimentary region at the 5′ end (“tag”) so that the product can be extracted and enriched via PCR. The tags are used to prevent the carry-over and accumulation of amplifications resulting from background amplifications (amplification of DNA polymerase sequences that were not selected for in the ePCR step but were carried over as parental plasmid DNA to recovery PCR).

Blocking oligos were designed to bind downstream of the CSR primer binding region, and had the 3′ end blocked with a C3 spacer to prevent extension from the 3′ end. Primers were designed taking into consideration the necessary Melting Temperatures, since one of the selective pressures was reduced extension temperature. Calculating the melting temperature and performing thermodynamic modelling for estimating the propensity of primers to hybridize with other primers or to hybridize at unintended sites in the template offer an accurate approach for predicting the energetic stability of DNA structures. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium).

The sequencing data from each mutant was analyzed to identify mutations in the nucleotide level, which were then translated to amino acids. The amino acid mutations calculated frequency of each mutation per round was obtained. The CSR Library was narrowed over the rounds of selection and shows many enriched mutations that are involved in strand-displacement. After each round of selection, the sequence of the enzyme was obtained to elucidate which mutations are responsible for the strand-displacement activity. Table 2 provides an overview of some of the mutations within the finger and palm subdomain responsible for increased strand displacing activity. Using the CSR techniques, novel mutations in the finger/palm subdomains in a DNA polymerase were found. For example, the mutations identified in over 30% of the mutant enzymes are identified in Table 2.

TABLE 2
Summary of the high frequency point mutations identified in strand-
displacing mutants; the point mutations are relative to SEQ ID NO: 1.
Point mutation

F588L
F588I
Q520H
E580G
K465E
A491V
K472E

### Example 2. Broader Mutational Analysis

Simultaneously, we extended the mutational analysis to the entire enzyme and not limiting it to a particular subdomain (e.g., the finger or palm subdomain). Prior to performing CSR, an error prone PCR library was generated with a target error rate of 8 to 15 mutations per clone using a mutant sequencing enzyme having the sequence of SEQ ID NO:1. The library was cloned using pET21b+ following standard restriction enzyme digestion and ligation as well as a commercial vector pD431-SR from ATUM via Gibson Assembly, using the Gibson Assembly® Master Mix (NEB Catalog Number E2611S), as described above.

Both libraries were subjected to 10 rounds of selective pressure, described in Table 3, wherein each round decreased the reaction temperature and/or increased the concentration of blocking oligos.

TABLE 3
Description of the selective pressure dimensions
utilized to develop strand-displacing variants.

| | Selection Pressure | | | | |
|---|---|---|---|---|---|
| Round | Denaturation | Extension | Extension | | Excess |
| # | Tr | temperature | time | | blocking oligos |
| Round 1 | 91° C. | 72° C. | 3 | min | 0 |
| Round 2 | 91° C. | 72° C. | 3 | min | 0 |
| Round 3 | 91° C. | 68° C. | 3 | min | 0 |
| Round 4 | 91° C. | 64° C. | 3 | min | 2× |
| Round 5 | 91° C. | 60° C. | 3 | min | 2× |
| Round 6 | 91° C. | 60° C. | 3 | min | 4× |
| Round 7 | 91° C. | 57° C. | 3 | min | 4× |
| Round 8 | 91° C. | 55° C. | 2 | min | 4× |
| Round 9 | 96° C. | 55° C. | 1 | min | 4× |
| Round 10 | 98° C. | 55° C. | 30 | seconds | 4× |

An additional selective pressure was applied in later rounds, decreasing the reaction time from 3 minutes to a total extension time of 30 seconds, to provide enzymes with strand-displacing activity and accelerated processivity. A total of 195 unique point mutations were observed, however, the mutations occurring at the highest frequency are provided in Table 4.

TABLE 4
Summary of some of the high frequency point
mutations identified in strand-displacing mutants;
the point mutations are relative to SEQ ID NO: 1.
Point mutations

| | |
|---|---|
| Y7H | A491V |
| K13R | R526H |
| A40V | E579G |
| F75L | F588L |
| R97C, H | T606I |
| G149D | G635D |
| K192R | V637D |
| K199E | N672D |
| F241I | H726D |
| M275L | V742A |
| A316S | F749Y |
| K395E | G765D |
| K469T | W769R |
| K472N | |

### Example 3. Strand Displacing Assay

Random clones from the library including one or more of the high-frequency mutations, as well as new variants based on the enriched mutations, were screened for strand-displacement activity. The polymerase chain displacement reaction (PCDR) assay was developed to measure the strand-displacing activity of the mutants, as described in Harris et al. BioTechniques 54:93-97 (February 2013), such that when extension occurs from the outer primer, it displaces the extension strand produced from the inner primer by utilizing a polymerase that has strand displacement activity. Briefly, the assay includes annealing two outer primers, PrimA and PrimD, to a template nucleic acid. Two or more inner primers are annealed to the template, Prim B and PrimC, wherein the primers hybridize to complementary regions between the outer primers. No strand-displacement activity is required to amplify from the inner primers (e.g., generating small fragments from PrimB to PrimC). Variants possessing strand-displacing activity are capable of generating amplification products from the outer primers and the inner primers at 50° C. and/or 61° C. The results of the strand-displacement assay are provided in Table 4. The variants examined included point mutations in SEQ ID NO:1 and/or SDS-0.

Further strand-displacing examination using a hairpin-challenge sequencing assay occurred for variants passing the initial PCDR assay to ensure the enzymes are capable of sequencing nucleic acid templates. Briefly, the hairpin-challenge strand-displacing assay utilizes streptavidin beads conjugated to a dual hairpin challenge template. A total of 9 different templates were used, varying the GC content within each of the templates. A sequencing primer anneals to the template, and in the presence of modified nucleotides (e.g., SBS nucleotides, wherein the nucleotides include a label and a reversible terminator), the hairpin templates were sequenced. A summary of the results are provided in Table 5.

TABLE 5
Results of the strand displacing assay. A ‘+’
is indicative of strand-displacing activity detection,
whereas a ‘−’ indicates no strand-displacing
activity was detected. The point mutations provided
below are relative to SEQ ID NO: 1.

| Mutation | SD products detect |
|---|---|
| Y7H | + |
| K13R | + |
| Q91H | + |
| R97H | + |
| R97C | + |
| G245S | + |
| R467C & K469T | − |
| E579G | + |
| F588L | + |
| E579G & F588L | + |
| H634Y | + |
| H726D | + |
| N23Y | + |
| F26S | + |
| V742A & F588L | + |
| Y7H & R97H | + |
| Y7H & R97C | + |
| D215G | + |
| D215E | + |
| W769R | + |
| W769L | + |
| F261Y | + |
| E581G | + |
| E581G & F588L | + |
| E581G & F588L & T591V | + |
| F588V | + |
| E581G & F588V | + |
| Q520H & E581G & F588L & T591V | + |
| Q520H & E581G & F588V | + |

TABLE 6
List of mutations in variant polymerases. The mutations in this table are
mutations relative to the wild type P. horikoshii (SEQ ID NO: 1).

| Internal Ref # | Amino acids |
|---|---|
| SDS-1 | M129A; D141A; E143A; T144A; L409A; Y410G; A486V; T515S; T591I; |
| | K477W; K478A; A640L |
| SDS-2 | M129A; D141A; E143A; T144A; L409A; Y410G; A486V; T515S; T591I; |
| | K477W; K478A; A640L; V93Q |
| SDS-3 | M129A; D141A; E143A; T144A; L409A; Y410G; A486V; T515S; A640L |
| SDS-4 | V93Q; M129A; D141A; E143A; T144A; G153E; L409A; Y410G; A486V; |
| | T515S; K477W; K478A; A640L |
| SDS-5 | R97H; M129A; D141A; E143A; T144A; L409A; Y410G; A486V; T515S; |
| | A640L |
| SDS-6 | R97L; M129A; D141A; E143A; T144A; L409A; Y410G; A486V; T515S; |
| | A640L |
| SDS-7 | R97S; M129A; D141A; E143A; T144A; L409A; Y410G; A486V; T515S; |
| | A640L |
| SDS-8 | P36Q; M129A; D141A; E143A; T144A; L409A; Y410G; A486V; T515S; |
| | A640L |
| SDS-9 | V93Q; R97H; M129A; D141A; E143A; T144A; L409A; Y410G; A486V; |
| | T515S; T591I; K477W; K478A; A640L |
| SDS-10 | V93Q; R97L; M129A; D141A; E143A; T144A; L409A; Y410G; A486V; |
| | T515S; T591I; K477W; K478A; A640L |
| SDS-11 | V93Q; R97S; M129A; D141A; E143A; T144A; L409A; Y410G; A486V; |
| | T515S; T591I; K477W; K478A; A640L |
| SDS-12 | V93Q; P36Q; M129A; D141A; E143A; T144A; L409A; Y410G; A486V; |
| | T515S; T591I; K477W; K478A; A640L |
| SDS-13 | M129A; D141A; E143A; T144A; L409A; Y410G; A486V; T515S; A640L; |
| | L766H |
| SDS-14 | M129A; D141A; E143A; T144A; L409A; Y410G; A486V; T515S; A640L; |
| | L766I |
| SDS-15 | M129A; D141A; E143A; T144A; L409A; Y410G; A486V; T515S; A640L; |
| | L766P |
| SDS-16 | M129A; D141A; E143A; T144A; L409A; Y410G; A486V; T515S; A640L; |
| | L766F |
| SDS-17 | I80V; V93A; M129A; D141A; E143A; T144A; L409A; Y410G; A486V; |
| | T515S; A640L |
| SDS-18 | I80V; E581V; M129A; D141A; E143A; T144A; L409A; Y410G; A486V; |
| | T515S; A640L |
| SDS-19 | V93A; E581V; M129A; D141A; E143A; T144A; L409A; Y410G; A486V; |
| | T515S; A640L |
| SDS-20 | V93Q; M129A; D141A; E143A; T144A; L409A; Y410G; A486V; T515S; |
| | A640L; C429S; C443S; C507S; C510S |
| SDS-21 | Q91H; V93Q; M129A; D141A; E143A; T144A; L409A; Y410G; C429S; |
| | C443S; A486V; C507S; C510S; T515S; A640L |
| SDS-22 | V93Q; R97H; M129A; D141A; E143A; T144A; L409A; Y410G; C429S; |
| | C443S; A486V; C507S; C510S; T515S; A640L |
| SDS-23 | V93Q; M129A; D141A; E143A; T144A; L409A; Y410G; C429S; C443S; |
| | A486V; C507S; C510S; T515S; E579G; A640L |
| SDS-24 | V93Q; M129A; D141A; E143A; T144A; L409A; Y410G; C429S; C443S; |
| | A486V; C507S; C510S; T515S; F588L; A640L |
| SDS-25 | V93Q; M129A; D141A; E143A; T144A; L409A; Y410G; C429S; C443S; |
| | A486V; C507S; C510S; T515S; E579G; F588L; A640L |
| SDS-26 | V93Q; M129A; D141A; E143A; T144A; L409A; Y410G; C429S; C443S; |
| | A486V; C507S; C510S; T515S; A640L; H634Y |
| SDS-27 | V93Q; M129A; D141A; E143A; T144A; L409A; Y410G; C429S; C443S; |
| | A486V; C507S; C510S; T515S; A640L; H726D |
| SDS-28 | V93Q; M129A; D141A; E143A; T144A; L409A; Y410G; C429S; C443S; |
| | A486V; C507S; C510S; T515S; A640L; V742A |
| SDS-29 | Y7H; V93Q; M129A; D141A; E143A; T144A; L409A; Y410G; C429S; |
| | C443S; A486V; C507S; C510S; T515S; A640L |
| SDS-30 | K13R; V93Q; M129A; D141A; E143A; T144A; L409A; Y410G; C429S; |
| | C443S; A486V; C507S; C510S; T515S; A640L |
| SDS-31 | V93Q; M129A; D141A; E143A; T144A; L409A; Y410G; C429S; C443S; |
| | A486V; C507S; C510S; T515S; A640L; W769R |
| SDS-32 | V93Q; M129A; D141A; E143A; T144A; L409A; Y410G; C429S; C443S; |
| | A486V; C507S; C510S; T515S; A640L; W769L |
| SDS-33 | V93Q; M129A; D141A; E143A; T144A; D245G; L409A; Y410G; C429S; |
| | C443S; A486V; C507S; C510S; T515S; A640L |
| SDS-34 | V93Q; M129A; D141A; E143A; T144A; G245S; L409A; Y410G; C429S; |
| | C443S; A486V; C507S; C510S; T515S; A640L |
| SDS-35 | V93Q; M129A; D141A; E143A; T144A; D246E; L409A; Y410G; C429S; |
| | C443S; A486V; C507S; C510S; T515S; A640L |
| SDS-36 | V93Q; M129A; D141A; E143A; T144A; G245S; D246E; L409A; Y410G; |
| | C429S; C443S; A486V; C507S; C510S; T515S; A640L |
| SDS-37 | V93Q; M129A; D141A; E143A; T144A; G350S; L409A; Y410G; C429S; |
| | C443S; A486V; C507S; C510S; T515S; A640L |
| SDS-38 | V93Q; M129A; D141A; E143A; T144A; L409A; Y410G; C429S; C443S; |
| | A486V; R467S; C507S; C510S; T515S; A640L |
| SDS-39 | V93Q; M129A; D141A; E143A; T144A; L409A; Y410G; C429S; C443S; |
| | A486V; K469T; C507S; C510S; T515S; A640L |
| SDS-40 | V93Q; M129A; D141A; E143A; T144A; L409A; Y410G; C429S; C443S; |
| | A486V; C507S; C510S; T515S; A640L; L766P |
| SDS-41 | V93Q; M129A; D141A; E143A; T144A; L409A; Y410G; C429S; C443S; |
| | A486V; C507S; C510S; T515S; A640L; A730E |
| SDS-42 | Y7H; V93Q; M129A; D141A; E143A; T144A; L409A; Y410G; C429S; |
| | C443S; A486V; C507S; C510S; T515S; E579G; F588L; A640L |
| SDS-43 | V93Q; M129A; D141A; E143A; T144A; L409A; Y410G; C429S; C443S; |
| | A486V; C507S; C510S; T515S; E579G; F588L; A640L; V742A |
| SDS-44 | K13R; V93Q; M129A; D141A; E143A; T144A; L409A; Y410G; C429S; |
| | C443S; A486V; C507S; C510S; T515S; E579G; F588L; A640L |
| SDS-45 | V93Q; R97H; M129A; D141A; E143A; T144A; L409A; Y410G; C429S; |
| | C443S; A486V; C507S; C510S; T515S; E579G; F588L; A640L |
| SDS-46 | Q91H; V93Q; M129A; D141A; E143A; T144A; L409A; Y410G; C429S; |
| | C443S; A486V; C507S; C510S; T515S; E579G; F588L; A640L |
| SDS-47 | V93Q; M129A; D141A; E143A; T144A; L409A; Y410G; C429S; C443S; |
| | A486V; C507S; C510S; T515S; E579G; F588L; A640L; H726D |
| SDS-48 | V93Q; M129A; D141A; E143A; T144A; L409A; Y410G; C429S; C443S; |
| | A486V; C507S; C510S; T515S; E579G; F588L; H634Y; A640L |
| SDS-49 | V93Q; M129A; D141A; E143A; T144A; L409A; Y410G; C429S; C443S; |
| | A486V; C507S; C510S; T515S; E579G; F588L; A640L; W769R |
| SDS-50 | V93Q; M129A; D141A; E143A; T144A; G245S; L409A; Y410G; C429S; |
| | C443S; A486V; C507S; C510S; T515S; E579G; F588L; A640L |
| SDS-51 | V93Q; M129A; D141A; E143A; T144A; L409A; Y410G; C429S; C443S; |
| | A486V; C507S; C510S; T515S; E581G; A640L |
| SDS-52 | V93Q; M129A; D141A; E143A; T144A; L409A; Y410G; C429S; C443S; |
| | A486V; C507S; C510S; T515S; E581G; F588L; A640L |
| SDS-53 | V93Q; M129A; D141A; E143A; T144A; L409A; Y410G; C429S; C443S; |
| | A486V; C507S; C510S; T515S; Q520H; E581G; F588L; A640L |
| SDS-54 | V93Q; M129A; D141A; E143A; T144A; L409A; Y410G; C429S; C443S; |
| | A486V; C507S; C510S; T515S; E581G; F588L; T591V; A640L |
| SDS-55 | V93Q; M129A; D141A; E143A; T144A; L409A; Y410G; C429S; C443S; |
| | A486V; C507S; C510S; T515S; Q520H; E581G; F588L; T591V; A640L |
| SDS-56 | V93Q; M129A; D141A; E143A; T144A; L409A; Y410G; C429S; C443S; |
| | A486V; C507S; C510S; T515S; F588V; A640L |
| SDS-57 | V93Q; M129A; D141A; E143A; T144A; L409A; Y410G; C429S; C443S; |
| | A486V; C507S; C510S; T515S; E581G; F588V; A640L |
| SDS-58 | V93Q; M129A; D141A; E143A; T144A; L409A; Y410G; C429S; C443S; |
| | A486V; C507S; C510S; T515S; Q520H; E581G; F588V; A640L |
| SDS-59 | V93Q; M129A; D141A; E143A; T144A; L409A; Y410G; C429S; C443S; |
| | A486V; C507S; C510S; T515S; Q520H; A640L |
| SDS-60 | V93Q; M129A; D141A; E143A; T144A; L409A; Y410G; C429S; C443S; |
| | K465E; A486V; C507S; C510S; T515S; A640L |
| SDS-61 | V93Q; M129A; D141A; E143A; T144A; L409A; Y410G; C429S; C443S; |
| | A486V; A491V; C507S; C510S; T515S; A640L |
| SDS-62 | V93Q; M129A; D141A; E143A; T144A; L409A; Y410G; C429S; C443S; |
| | R467S; K469T; A486V; C507S; C510S; T515S; A640L |
| SDS-63 | N23Y; V93Q; M129A; D141A; E143A; T144A; L409A; Y410G; C429S; |
| | C443S; A486V; C507S; C510S; T515S; E579G; F588L; A640L |
| SDS-64 | V93Q; M129A; D141A; E143A; T144A; F214I; L409A; Y410G; C429S; |
| | C443S; A486V; C507S; C510S; T515S; F588L; A640L |
| SDS-65 | F75L; V93Q; M129A; D141A; E143A; T144A; L409A; Y410G; C429S; |
| | C443S; A486V; C507S; C510S; T515S; F588L; A640L |
| SDS-66 | V93Q; M129A; D141A; E143A; T144A; L409A; Y410G; C429S; C443S; |
| | A486V; C507S; C510S; T515S; F588L; T606I; A640L |
| SDS-67 | V93Q; M129A; D141A; E143A; T144A; L409A; Y410G; C429S; C443S; |
| | A486V; C507S; C510S; T515S; R526H; F588L; A640L |
| SDS-68 | V93Q; M129A; D141A; E143A; T144A; L275P; L409A; Y410G; C429S; |
| | C443S; A486V; C507S; C510S; T515S; F588L; A640L |
| SDS-69 | V93Q; M129A; D141A; E143A; T144A; K395E; L409A; Y410G; C429S; |
| | C443S; A486V; C507S; C510S; T515S; F588L; A640L |
| SDS-70 | V93Q; M129A; D141A; E143A; T144A; L409A; Y410G; C429S; C443S; |
| | A486V; C507S; C510S; T515S; F588L; A640L; F749Y |
| SDS-71 | V93Q; M129A; D141A; E143A; T144A; L409A; Y410G; C429S; C443S; |
| | A486V; C507S; C510S; T515S; F588L; A640L; N672D |
| SDS-72 | V93Q; M129A; D141A; E143A; T144A; L409A; Y410G; C429S; C443S; |
| | A486V; C507S; C510S; T515S; F588L; V637D; A640L |
| SDS-73 | V93Q; M129A; D141A; E143A; T144A; A316S; L409A; Y410G; C429S; |
| | C443S; A486V; C507S; C510S; T515S; F588L; A640L |
| SDS-74 | V93Q; M129A; D141A; E143A; T144A; K192R; L409A; Y410G; C429S; |
| | C443S; A486V; C507S; C510S; T515S; F588L; A640L |
| SDS-75 | V93Q; M129A; D141A; E143A; T144A; K199E; L409A; Y410G; C429S; |
| | C443S; A486V; C507S; C510S; T515S; F588L; A640L |
| SDS-76 | V93Q; M129A; D141A; E143A; T144A; L409A; Y410G; C429S; C443S; |
| | K472N; A486V; C507S; C510S; T515S; F588L; A640L |
| SDS-77 | V93Q; M129A; D141A; E143A; T144A; L409A; Y410G; C429S; C443S; |
| | A486V; K469T; C507S; C510S; T515S; F588L; A640L |
| SDS-78 | V93Q; M129A; D141A; E143A; T144A; L409A; Y410G; C429S; C443S; |
| | A486V; C507S; C510S; T515S; F588L; G635D; A640L |
| SDS-79 | V93Q; M129A; D141A; E143A; T144A; G149D; L409A; Y410G; C429S; |
| | C443S; A486V; C507S; C510S; T515S; F588L; A640L |
| SDS-80 | V93Q; M129A; D141A; E143A; T144A; L409A; Y410G; C429S; C443S; |
| | A486V; C507S; C510S; T515S; F588L; A640L; G765D |
| SDS-81 | A40V; V93Q; M129A; D141A; E143A; T144A; L409A; Y410G; C429S; |
| | C443S; A486V; C507S; C510S; T515S; F588L; A640L |
| SDS-82 | I18V; V93Q; M129A; D141A; E143A; T144A; L409A; Y410G; C429S; |
| | C443S; A486V; C507S; C510S; T515S; F588L; A640L |
| SDS-83 | V93Q; M129A; D141A; E143A; T144A; I268F; L409A; Y410G; C429S; |
| | C443S; A486V; C507S; C510S; T515S; F588L; A640L |
| SDS-84 | V93Q; F116I; M129A; D141A; E143A; T144A; L409A; Y410G; C429S; |
| | C443S; A486V; C507S; C510S; T515S; F588L; A640L |
| SDS-85 | V93Q; R97H; M129A; D141A; E143A; T144A; L409A; Y410G; C429S; |
| | C443S; A486V; C507S; C510S; T515S; F588L; A640L |
| SDS-86 | V93Q; M129A; D141A; E143A; T144A; L409A; Y410G; C429S; C443S; |
| | A486V; C507S; C510S; T515S; Q520H; F588L; A640L |
| SDS-87 | V93Q; M129A; D141A; E143A; T144A; L409A; Y410G; C429S; C443S; |
| | K465E; A486V; C507S; C510S; T515S; F588L; A640L |
| SDS-88 | V93Q; M129A; D141A; E143A; T144A; L409A; Y410G; C429S; C443S; |
| | A486V; A491V; C507S; C510S; T515S; F588L; A640L |
| SDS-89 | Q91H; V93Q; M129A; D141A; E143A; T144A; L409A; Y410G; C429S; |
| | C443S; A486V; C507S; C510S; T515S; F588L; A640L |
| SDS-90 | K13R; V93Q; M129A; D141A; E143A; T144A; L409A; Y410G; C429S; |
| | C443S; A486V; C507S; C510S; T515S; F588L; A640L |
| SDS-91 | V93Q; M129A; D141A; E143A; T144A; L409A; Y410G; C429S; C443S; |
| | A486V; C507S; C510S; T515S; F588L; A640L; W769R |
| SDS-92 | V93Q; M129A; D141A; E143A; T144A; F261Y; L409A; Y410G; C429S; |
| | C443S; A486V; C507S; C510S; T515S; F588L; A640L |
| SDS-93 | V93Q; M129A; D141A; E143A; T144A; T349D; L409A; Y410G; C429S; |
| | C443S; A486V; C507S; C510S; T515S; A640L |
| SDS-94 | V93Q; M129A; D141A; E143A; T144A; T349V; L409A; Y410G; C429S; |
| | C443S; A486V; C507S; C510S; T515S; A640L |
| SDS-95 | V93Q; M129A; D141A; E143A; T144A; T349L; L409A; Y410G; C429S; |
| | C443S; A486V; C507S; C510S; T515S; A640L |
| SDS-96 | V93Q; M129A; D141A; E143A; T144A; T349I; L409A; Y410G; C429S; |
| | C443S; A486V; C507S; C510S; T515S; A640L |
| SDS-97 | V93Q; M129A; D141A; E143A; T144A; T349F; L409A; Y410G; C429S; |
| | C443S; A486V; C507S; C510S; T515S; A640L |
| SDS-98 | V93Q; M129A; D141A; E143A; T144A; T349A; L409A; Y410G; C429S; |
| | C443S; A486V; C507S; C510S; T515S; A640L |
| SDS-99 | V93Q; M129A; D141A; E143A; T144A; L409A; Y410G; C429S; C443S; |
| | A486V; K502L; C507S; C510S; T515S; A640L |
| SDS-100 | V93Q; M129A; D141A; E143A; T144A; L409A; Y410G; C429S; C443S; |
| | A486V; K502F; C507S; C510S; T515S; A640L |
| SDS-101 | V93Q; M129A; D141A; E143A; T144A; L409A; Y410G; C429S; C443S; |
| | A486V; K502G; C507S; C510S; T515S; A640L |
| SDS-102 | V93Q; M129A; D141A; E143A; T144A; L409A; Y410G; C429S; C443S; |
| | A486V; K502R; C507S; C510S; T515S; A640L |
| SDS-103 | V93Q; M129A; D141A; E143A; T144A; L270M; L409A; Y410G; C429S; |
| | C443S; A486V; C507S; C510S; T515S; A640L |
| SDS-104 | V93Q; M129A; D141A; E143A; T144A; L409A; Y410G; C429S; C443S; |
| | A486V; C507S; C510S; T515S; A640L; D646E |
| SDS-105 | V93Q; M129A; D141A; E143A; T144A; L409A; Y410G; C429S; C443S; |
| | A486V; C507S; C510S; T515S; F588L; A640L; V742A |
| SDS-106 | V93Q; M129A; D141A; E143A; T144A; L409A; Y410G; C429S; C443S; |
| | A486V; C507S; C510S; T515S; F588L; H634Y; A640L |
| SDS-107 | V93Q; M129A; D141A; E143A; T144A; L409A; Y410G; C429S; C443S; |
| | A486V; C507S; C510S; T515S; F588L; A640L; H726D |
| SDS-108 | Y7H; K13R; L76P; K192E; K289T; R364C; L397M; Q484H; E563G; |
| | F588L; V637D; R706H; V742A; L766P |
| SDS-109 | Y7H; K13R; L76P; PV93Q; M129A; D141A; E143A; T144A; K192E; |
| | K289T; L397M; L409A; Y410G; C429S; C443S; Q484H; A486V; C507S; |
| | C510S; T515S; E563G; F588L; V637D; A640L; R706H; V742A; L766P |

Though one parameter, the average half time of nucleotide incorporation is measured over all four nucleotides (A, T, C, and G), and serves as a useful indicator of the enzyme kinetics. Described in Table 7 is the percent change in the average halftime, t_{1/2} averaged over each of the four incorporated nucleotides (i.e., A, T, C, and G). An incorporation halftime was measured for a control, SDS-0, bearing the point mutations: V93Q, M129A, D141A, E143A, T144A, L409A, Y410G, A486V, T515S, A640L, C429S, C443S, C507S, and C510S. The percent change was calculated as the difference in average halftimes divided by the reference halftime, or T(variant)-T(SDS-0)/T(SDS-0), wherein T is the average halftime. Reactions were initiated in a house-developed buffer by the addition of 300 nM nucleotides and 133 nM DNA polymerase at a temperature of 65° C. A negative percent change indicates an improvement in the incorporation kinetics relative to SDS-0, whereas a positive perfect change implies slower incorporation kinetics under the observed experimental conditions. For example, some point mutations relative to SDS-0 are found in SDS-95 (T349L); SDS-24 (F588L); SDS-60 (K465E); SDS-86 (Q520L and F588L); SDS-65 (F75L and F588L); SDS-63 (N23Y and F588L); and SDS-66 (T6061 and F588L) and were found to significantly improve the incorporation kinetics while having measurable strand-displacing activity. Some of the variants displayed enhanced strand displacement, albeit with slower nucleotide incorporation. This tradeoff should be evaluated on a case-by-case basis.

TABLE 7
The percent change of the average half time of incorporation of labeled
nucleotides bearing a reversible terminator moiety sing a strand-
displacing variant as described herein relative to a control (i.e.,
SDS-0). The percent change was calculated as T(variant) − T(SDS-
0)/T(SDS-0), wherein T is the measured average halftime.

| Internal Ref # | % Change |
|---|---|
| SDS-95 | −55% |
| SDS-24 | −47% |
| SDS-60 | −43% |
| SDS-86 | −39% |
| SDS-65 | −32% |
| SDS-63 | −32% |
| SDS-66 | −28% |
| SDS-59 | −16% |
| SDS-22 | −16% |
| SDS-58 | −12% |
| SDS-67 | −9% |
| SDS-21 | −7% |
| SDS-4 | −7% |
| SDS-12 | 0% |
| SDS-20 | 0% |
| SDS-77 | 0% |
| SDS-56 | 2% |
| SDS-1 | 4% |
| SDS-61 | 6% |
| SDS-30 | 7% |
| SDS-36 | 7% |
| SDS-26 | 9% |
| SDS-99 | 10% |
| SDS-53 | 11% |
| SDS-31 | 12% |
| SDS-83 | 13% |
| SDS-91 | 13% |
| SDS-75 | 17% |
| SDS-32 | 17% |
| SDS-80 | 19% |
| SDS-104 | 23% |
| SDS-52 | 28% |
| SDS-40 | 29% |
| SDS-87 | 32% |
| SDS-44 | 35% |
| SDS-84 | 36% |
| SDS-15 | 37% |
| SDS-29 | 39% |
| SDS-94 | 43% |
| SDS-19 | 43% |
| SDS-2 | 45% |
| SDS-5 | 45% |
| SDS-88 | 47% |
| SDS-57 | 52% |
| SDS-38 | 54% |
| SDS-18 | 54% |
| SDS-54 | 57% |
| SDS-64 | 59% |
| SDS-92 | 59% |
| SDS-106 | 59% |
| SDS-28 | 61% |
| SDS-9 | 62% |
| SDS-100 | 64% |
| SDS-85 | 64% |
| SDS-11 | 67% |
| SDS-14 | 73% |
| SDS-10 | 75% |
| SDS-79 | 80% |
| SDS-62 | 81% |
| SDS-82 | 81% |
| SDS-107 | 81% |
| SDS-8 | 87% |
| SDS-51 | 89% |
| SDS-16 | 91% |
| SDS-73 | 104% |
| SDS-39 | 106% |
| SDS-45 | 109% |
| SDS-50 | 110% |
| SDS-49 | 122% |
| SDS-17 | 159% |
| SDS-96 | 193% |
| SDS-27 | 195% |
| SDS-47 | 200% |
| SDS-3 | 215% |
| SDS-101 | 230% |
| SDS-48 | 240% |
| SDS-70 | 240% |
| SDS-102 | 240% |
| SDS-55 | >250% |
| SDS-23 | >250% |
| SDS-25 | >250% |
| SDS-43 | >250% |
| SDS-103 | >250% |
| SDS-93 | >250% |
| SDS-71 | >250% |
| SDS-42 | >250% |
| SDS-46 | >250% |

SEQUENCES

Amino Acid Sequence of wild type P. horikoshii OT3 (SEQ ID NO: 1):
MILDADYITEDGKPIIRIFKKENGEFKVEYDRNFRPYIYALLRDDSAIDEIKKITAQRHGKVVRIVET
EKIQRKFLGRPIEVWKLYLEHPQDVPAIRDKIREHPAVVDIFEYDIPFAKRYLIDKGLTPMEGNEKLT
FLAVDIETLYHEGEEFGKGPVIMISYADEEGAKVITWKKIDLPYVEVVSSEREMIKRLIRVIKEKDPD
VIITYNGDNFDFPYLLKRAEKLGIKLLLGRDNSEPKMQKMGDSLAVEIKGRIHFDLFPVIRRTINLPT
YTLEAVYEAIFGKPKEKVYADEIAKAWETGEGLERVAKYSMEDAKVTYELGREFFPMEAQLARLVGQP
VWDVSRSSTGNLVEWFLLRKAYERNELAPNKPDEKEYERRLRESYEGGYVKEPEKGLWEGIVSLDFRS
LYPSIIITHNVSPDTLNREGCEEYDVAPKVGHRFCKDFPGFIPSLLGQLLEERQKIKKRMKESKDPVE
KKLLDYRQRAIKILANSYYGYYGYAKARWYCKECAESVTAWGRQYIDLVRRELEARGFKVLYIDTDGL
YATIPGVKDWEEVKRRALEFVDYINSKLPGVLELEYEGFYARGFFVTKKKYALIDEEGKIVTRGLEIV
RRDWSEIAKETQARVLEAILKHGNVEEAVKIVKDVTEKLTNYEVPPEKLVIYEQITRPINEYKAIGPH
VAVAKRLMARGIKVKPGMVIGYIVLRGDGPISKRAISIEEFDPRKHKYDAEYYIENQVLPAVERILKA
FGYKREDLRWQKTKQVGLGAWIKVKKS
DNA Sequence of wild type P. horikoshii OT3 (SEQ ID NO: 2):
Pyrococcus horikoshii DNA Polymerase gene
ATGATTCTGGACGCTGATTATATTACTGAAGATGGTAAACCGATTATTCGTATTTTTAAAAAAGAAAA
TGGCGAGTTCAAAGTTGAATATGACCGTAACTTTCGTCCGTACATCTACGCGCTGTTGCGCGACGATA
GCGCGATCGATGAGATTAAGAAAATTACCGCGCAGCGTCATGGTAAAGTTGTTCGCATCGTTGAAACC
GAGAAAATTCAACGTAAATTCCTGGGCCGCCCAATTGAAGTGTGGAAGCTGTACCTGGAGCATCCGCA
AGATGTCCCGGCGATCCGTGACAAGATTCGCGAGCACCCGGCCGTCGTCGACATTTTCGAATACGATA
TTCCGTTCGCAAAGCGTTACCTGATCGATAAGGGTCTGACCCCGATGGAGGGTAATGAAAAGCTGACG
TTCCTGGCTGTCGATATTGAAACGTTGTACCACGAGGGTGAAGAGTTTGGTAAGGGCCCGGTCATTAT
GATCAGCTACGCGGATGAAGAGGGCGCCAAAGTTATCACGTGGAAAAAAATTGATCTGCCGTACGTTG
AAGTTGTGTCCAGCGAGCGCGAGATGATTAAACGCTTGATTCGTGTGATTAAAGAAAAAGATCCAGAC
GTGATCATTACCTATAATGGTGACAACTTTGACTTTCCGTACTTGCTGAAACGTGCTGAGAAACTGGG
TATCAAGCTGTTGCTGGGTCGCGATAATAGCGAGCCGAAGATGCAAAAAATGGGCGATAGCCTGGCAG
TCGAGATCAAGGGTCGCATCCACTTTGATCTCTTTCCGGTGATTCGTCGCACGATCAATCTGCCGACC
TATACGCTGGAAGCTGTCTACGAGGCAATCTTTGGTAAGCCGAAAGAAAAAGTCTATGCGGACGAAAT
TGCGAAAGCGTGGGAAACCGGCGAGGGCCTGGAGCGTGTGGCAAAGTACTCTATGGAAGATGCCAAAG
TGACCTATGAACTGGGTCGTGAGTTCTTCCCAATGGAAGCCCAGTTGGCGCGCTTGGTGGGCCAACCG
GTTTGGGACGTTTCCCGTAGCAGCACCGGTAACCTGGTTGAGTGGTTTCTGTTGCGTAAAGCGTATGA
GCGTAATGAACTGGCACCGAACAAGCCTGACGAGAAAGAATATGAACGTCGCCTGCGTGAATCTTACG
AGGGTGGTTACGTCAAAGAACCGGAAAAGGGTCTGTGGGAAGGCATCGTGAGCCTGGATTTCCGTAGC
CTGTACCCTAGCATCATCATCACGCACAATGTTAGCCCGGACACCCTGAACCGCGAGGGCTGCGAAGA
GTACGACGTTGCGCCGAAAGTCGGCCATCGTTTTTGTAAAGACTTCCCTGGTTTCATCCCAAGCCTGC
TGGGTCAGCTGCTGGAAGAGAGACAGAAAATTAAAAAACGCATGAAAGAATCGAAAGATCCGGTTGAG
AAAAAGCTGCTGGATTACCGCCAGCGTGCCATCAAGATTCTGGCTAACTCATATTATGGCTACTACGG
TTATGCTAAAGCGCGTTGGTACTGTAAAGAGTGCGCGGAGTCCGTCACCGCGTGGGGTCGCCAGTATA
TCGATCTGGTGCGTCGCGAGCTGGAAGCGCGTGGTTTTAAGGTCCTGTACATCGATACTGACGGTCTG
TATGCAACCATCCCTGGTGTCAAAGACTGGGAAGAGGTTAAGCGTCGTGCACTGGAATTTGTGGACTA
TATCAATTCTAAGTTGCCGGGTGTGCTGGAGCTGGAGTACGAAGGCTTCTATGCACGCGGCTTTTTCG
TTACGAAAAAGAAATACGCACTGATCGACGAAGAGGGCAAGATTGTGACTCGTGGTCTGGAAATCGTT
CGTCGCGACTGGAGCGAGATTGCAAAAGAAACCCAAGCTCGCGTTCTGGAAGCAATCCTGAAACATGG
TAACGTCGAAGAAGCCGTCAAGATCGTGAAAGATGTCACCGAAAAGTTGACCAACTACGAAGTTCCAC
CGGAAAAACTGGTGATTTATGAGCAAATCACGCGTCCGATCAATGAATATAAGGCCATTGGCCCGCAC
GTCGCGGTGGCCAAGCGCCTGATGGCGCGTGGTATCAAAGTGAAACCGGGTATGGTTATTGGTTACAT
CGTGCTGCGTGGCGACGGCCCGATTAGCAAACGTGCGATCAGCATTGAAGAATTTGACCCGCGTAAGC
ACAAATATGACGCGGAATACTATATCGAGAATCAAGTGCTGCCGGCCGTGGAACGCATTCTGAAAGCT
TTCGGCTACAAGCGTGAAGATTTGCGCTGGCAGAAAACCAAACAGGTTGGTCTTGGTGCGTGGATCAA
GGTCAAAAAGTCCTAA
Pyrococcus abyssi (SEQ ID NO: 3)
MIIDADYITEDGKPIIRIFKKEKGEFKVEYDRTFRPYIYALLKDDSAIDEVKKITAERHGKIVRITEV
EKVQKKFLGRPIEVWKLYLEHPQDVPAIREKIREHPAVVDIFEYDIPFAKRYLIDKGLTPMEGNEELT
FLAVDIETLYHEGEEFGKGPIIMISYADEEGAKVITWKSIDLPYVEVVSSEREMIKRLVKVIREKDPD
VIITYNGDNFDFPYLLKRAEKLGIKLPLGRDNSEPKMQRMGDSLAVEIKGRIHFDLFPVIRRTINLPT
YTLEAVYEAIFGKSKEKVYAHEIAEAWETGKGLERVAKYSMEDAKVTFELGKEFFPMEAQLARLVGQP
VWDVSRSSTGNLVEWFLLRKAYERNELAPNKPDEREYERRLRESYEGGYVKEPEKGLWEGIVSLDFRS
LYPSIIITHNVSPDTLNRENCKEYDVAPQVGHRFCKDFPGFIPSLLGNLLEERQKIKKRMKESKDPVE
KKLLDYRQRAIKILANSYYGYYGYAKARWYCKECAESVTAWGRQYIDLVRRELESRGFKVLYIDTDGL
YATIPGAKHEEIKEKALKFVEYINSKLPGLLELEYEGFYARGFFVTKKKYALIDEEGKIVTRGLEIVR
RDWSEIAKETQAKVLEAILKHGNVDEAVKIVKEVTEKLSKYEIPPEKLVIYEQITRPLSEYKAIGPHV
AVAKRLAAKGVKVKPGMVIGYIVLRGDGPISKRAIAIEEFDPKKHKYDAEYYIENQVLPAVERILRAF
GYRKEDLKYQKTKQVGLGAWLKF
Pyrococcus woesei (SEQ ID NO: 4)
MILDVDYITEEGKPVIRLFKKENGKFKIEHDRTFRPYIYALLRDDSKIEEVKKITGERHGKIVRIVDV
EKVEKKFLGKPITVWKLYLEHPQDVPTIREKVREHPAVVDIFEYDIPFAKRYLIDKGLIPMEGEEELK
ILAFDIETLYHEGEEFGKGPIIMISYADENEAKVITWKNIDLPYVEVVSSEREMIKRFLRIIREKDPD
IIVTYNGDSFDFPYLAKRAEKLGIKLTIGRDGSEPKMQRIGDMTAVEVKGRIHFDLYHVITRTINLPT
YTLEAVYEAIFGKPKEKVYADEIAKAWESGENLERVAKYSMEDAKATYELGKEFLPMEIQLSRLVGQP
LWDVSRSSTGNLVEWFLLRKAYERNEVAPNKPSEEEYQRRLRESYTGGFVKEPEKGLWENIVYLDFRA
LYPSIIITHNVSPDTLNLEGCKNYDIAPQVGHKFCKDIPGFIPSLLGHLLEERQKIKTKMKETQDPIE
KILLDYRQKAIKLLANSFYGYYGYAKARWYCKECAESVTAWGRKYIELVWKELEEKFGFKVLYIDTDG
LYATIPGGESEEIKKKALEFVKYINSKLPGLLELEYEGFYKRGFFVTKKRYAVIDEEGKVITRGLEIV
RRDWSEIAKETQARVLETILKHGDVEEAVRIVKEVIQKLANYEIPPEKLAIYEQITRPLHEYKAIGPH
VAVAKKLAAKGVKIKPGMVIGYIVLRGDGPISNRAILAEEYDPKKHKYDAEYYIENQVLPAVLRILEG
FGYRKEDLRYQKTRQVGLTSWLNIKKS
Pyrococcus furiosus (SEQ ID NO: 5)
MILDVDYITEEGKPVIRLFKKENGKFKIEHDRTFRPYIYALLRDDSKIEEVKKITGERHGKIVRIVDV
EKVEKKFLGKPITVWKLYLEHPQDVPTIREKVREHPAVVDIFEYDIPFAKRYLIDKGLIPMEGEEELK
ILAFDIETLYHEGEEFGKGPIIMISYADENEAKVITWKNIDLPYVEVVSSEREMIKRFLRIIREKDPD
IIVTYNGDSFDFPYLAKRAEKLGIKLTIGRDGSEPKMQRIGDMTAVEVKGRIHFDLYHVITRTINLPT
YTLEAVYEAIFGKPKEKVYADEIAKAWESGENLERVAKYSMEDAKATYELGKEFLPMEIQLSRLVGQP
LWDVSRSSTGNLVEWFLLRKAYERNEVAPNKPSEEEYQRRLRESYTGGFVKEPEKGLWENIVYLDFRA
LYPSIIITHNVSPDTLNLEGCKNYDIAPQVGHKFCKDIPGFIPSLLGHLLEERQKIKTKMKETQDPIE
KILLDYRQKAIKLLANSFYGYYGYAKARWYCKECAESVTAWGRKYIELVWKELEEKFGFKVLYIDTDG
LYATIPGGESEEIKKKALEFVKYINSKLPGLLELEYEGFYKRGFFVTKKRYAVIDEEGKVITRGLEIV
RRDWSEIAKETQARVLETILKHGDVEEAVRIVKEVIQKLANYEIPPEKLAIYEQITRPLHEYKAIGPH
VAVAKKLAAKGVKIKPGMVIGYIVLRGDGPISNRAILAEEYDPKKHKYDAEYYIENQVLPAVLRILEG
FGYRKEDLRYQKTRQVGLTSWLNIKKS
Pyrococcus glycovorans (SEQ ID NO: 6)
MILDADYITEDGKPIIRIFKKENGEFKVEYDRNFRPYIYALLKDDSQIDEVKKITAERHGKIVRIVDV
EKVKKKFLGRPIEVWKLYFEHPQDVPAIRDKIREHPAVVDIFEYDIPFAKRYLIDKGLIPMEGDEELK
LLAFDIETLYHEGEEFAKGPIIMISYADEEGAKVITWKKVDLPYVEVVSSEREMIKRFLKVIREKDPD
VIITYNGDSFDLPYLVKRAEKLGIKLPLGRDGSEPKMQRLGDMTAVEIKGRIHFDLYHVIRRTINLPT
YTLEAVYEAIFGKPKEKVYAHEIAEAWETGKGLERVAKYSMEDAKVTYELGREFFPMEAQLSRLVGQP
LWDVSRSSTGNLVEWYLLRKAYERNELAPNKPDEREYERRLRESYAGGYVKEPEKGLWEGLVSLDFRS
LYPSIIITHNVSPDTLNREGCMEYDVAPEVKHKFCKDFPGFIPSLLKRLLDERQEIKRRMKASKDPIE
KKMLDYRQRAIKILANSYYGYYGYAKARWYCKECAESVTAWGREYIEFVRKELEEKFGFKVLYIDTDG
LYATIPGAKPEEIKRKALEFVEYINAKLPGLLELEYEGFYVRGFFVTKKKYALIDEEGKIITRGLEIV
RRDWSEIAKETQAKVLEAILKHGNVEEAVKIVKEVTEKLSKYEIPPEKLVIYEQITRPLHEYKAIGPH
VAVAKRLAARGVKVRPGMVIGYIVLRGDGPISKRAILAEEFDPRKHKYDAEYYIENQVLPAVLRILEA
FGYRKEDLRWQKTKQTGLTAWLNVKKK
Pyrococcus sp. NA2 (SEQ ID NO: 7)
MILDADYITEDGKPIIRLFKKENGRFKVEYDRNFRPYIYALLKDDSAIDDVRKITSERHGKVVRVIDV
EKVKKKFLGRPIEVWKLYFEHPQDVPAMRDKIREHPAVIDIFEYDIPFAKRYLIDKGLIPMEGNEELT
FLAVDIETLYHEGEEFGKGPIIMISYADEEGAKVITWKKIDLPYVEVVANEREMIKRLIKVIREKDPD
VIITYNGDNFDFPYLLKRAEKLGMKLPLGRDNSEPKMQRLGDSLAVEIKGRIHFDLFPVIRRTINLPT
YTLEAVYEAIFGKQKEKVYPHEIAEAWETGKGLERVAKYSMEDAKVTYELGKEFFPMEAQLARLVGQP
LWDVSRSSTGNLVEWYLLRKAYERNELAPNKPDEREYERRLRESYEGGYVKEPERGLWEGIVSLDFRS
LYPSIIITHNVSPDTLNKEGCGEYDEAPEVGHRFCKDFPGFIPSLLGSLLEERQKIKKRMKESKDPVE
RKLLDYRQRAIKILANSFYGYYGYAKARWYCKECAESVTAWGRQYIELVRRELEERGFKVLYIDTDGL
YATIPGEKNWEEIKRRALEFVNYINSKLPGILELEYEGFYTRGFFVTKKKYALIDEEGKIVTRGLEIV
RRDWSEIAKETQAKVLEAILKHGNVEEAVKIVKEVTEKLSNYEIPVEKLVIYEQITRPLNEYKAIGPH
VAVAKRLAAKGIKIKPGMVIGYVVLRGDGPISKRAIAIEEFDGKKHKYDAEYYIENQVLPAVERILKA
FGYKREDLRWQKTKQVGLGAWLKVKKS
Pyrococcus sp. ST700 (SEQ ID NO: 8)
MILDADYITENGKPIIRLFKKENGKFKVEYDRNFRPYIYALLKDDSAIDDVRKITSERHGKVVRVIDV
EKVSKKFLGRPIEVWKLYFEHPQDVPAIRDKIREHPAVIDIFEYDIPFAKRYLIDKGLIPMEGNEELS
FLAVDIETLYHEGEEFGKGPIIMISYADEEGAKVITWKKIDLPYVEVVANEREMIKRLVRIIREKDPD
IIITYNGDNFDFPYLLKRAEKLGIKLPLGRDNSEPKMQRLGESLAVEIKGRIHFDLFPVIRRTINLPT
YTLRTVYEAIFGKPKEKVYPHEIAEAWETGKGLERVAKYSMEDAKVTYELGKEFFPMEAQLARLVGQP
VWDVSRSSTGNLVEWFLLRKAYERNELAPNKPDEKEYEKRLRESYEGGYVKEPEKGLWEGIVSLDFRS
LYPSIIITHNVSPDTLNREGCGKYDEAPEVGHRFCKDFPGFIPSLLGDLLEERQKIKKRMKESKDPIE
KKLLDYRQRAIKILANSFYGYYGYAKARWYCKECAESVTAWGRQYIELVRRELEERGFKVLYIDTDGL
YATIPGEKNWEEIKRKALEFVNYINSKLPGILELEYEGFYTRGFFVTKKKYALIDEEGKIITRGLEIV
RRDWSEIAKETQAKVLEAILKHGNVEEAVKIVKEVTEKLSNYEIPVEKLVIYEQITRPLNEYKAIGPH
VAVAKRLAAKGIKIKPGMVIGYVLLRGDGPISKRAIAIEEFDGKKHKYDAEYYIENQVLPAVERILKA
FGYKKEDLRWQ
Pyrococcus kukulkanii (SEQ ID NO: 9)
MILDADYITEDGKPIIRIFKKENGEFKVEYDRNFRPYIYALLKDDSQIDEVRKITAERHGKIVRIIDA
EKVRKKFLGRPIEVWRLYFEHPQDVPAIRDKIREHSAVIDIFEYDIPFAKRYLIDKGLIPMEGDEELK
LLAFDIETLYHEGEEFAKGPIIMISYADEEEAKVITWKKIDLPYVEVVSSEREMIKRFLKVIREKDPD
VIITYNGDSFDLPYLVKRAEKLGIKLPLGRDGSEPKMQRLGDMTAVEIKGRIHFDLYHVIRRTINLPT
YTLEAVYEAIFGKPKEKVYAHEIAEAWETGKGLERVAKYSMEDAKVTYELGREFFPMEAQLSRLVGQP
LWDVSRSSTGNLVEWYLLRKAYERNELAPNKPDEREYERRLRESYAGGYVKEPEKGLWEGLVSLDFRS
LYPSIIITHNVSPDTLNREGCREYDVAPEVGHKFCKDFPGFIPSLLKRLLDERQEIKRKMKASKDPIE
KKMLDYRQRAIKILANSYYGYYGYAKARWYCKECAESVTAWGREYIEFVRKELEEKFGFKVLYIDTDG
LYATIPGAKPEEIKKKALEFVDYINAKLPGLLELEYEGFYVRGFFVTKKKYALIDEEGKIITRGLEIV
RRDWSEIAKETQAKVLEAILKHGNVEEAVKIVKEVTEKLSKYEIPPEKLVIYEQITRPLHEYKAIGPH
VAVAKRLAARGVKVRPGMVIGYIVLRGDGPISKRAILAEEFDLRKHKYDAEYYIENQVLPAVLRILEA
FGYRKEDLRWQKTKQTGLTAWLNIKKK
Pyrococcus yayanosii (SEQ ID NO: 10)
MILDADYITENGKPVVRIFKKENGEFKVEYDRSFRPYIYALLRDDSAIEDIKKITAERHGKVVRVVEA
EKVRKKFLGRPIEVWKLYFEHPQDVPAIREKIREHPAVIDIFEYDIPFAKRYLIDKGLIPMEGNEELK
LLAFDIETLYHEGDEFGSGPIIMISYADEKGAKVITWKGVDLPYVEVVSSEREMIKRFLRVIREKDPD
VIITYNGDNFDFPYLLKRAEKLGMKLPIGRDGSEPKMQRMGDGFAVEVKGRIHFDIYPVIRRTINLPT
YTLEAVYEAVFGRPKEKVYPNEIARAWENCKGLERVAKYSMEDAKVTYELGREFFPMEAQLARLVGQP
VWDVSRSSTGNLVEWFLLRKAYERNELAPNRPDEREYERRLRESYEGGYVKEPEKGLWEGIIYLDFRS
LYPSIIITHNISPDTLNKEGCNSYDVAPKVGHRFCKDFPGFIPSLLGQLLDERQKIKRKMKATIDPIE
RKLLDYRQRAIKILANSYYGYYGYAKARWYCKECAESVTAWGREYIELVSRELEKRGFKVLYIDTDGL
YATIPGSREWDKIKERALEFVKYINARLPGLLELEYEGFYKRGFFVTKKKYALIDEEGKIITRGLEIV
RRDWSEIAKETQARVLEAILKEGNLEKAVKIVKEVTEKLSKYEVPPEKLVIYEQITRDLKDYKAVGPH
VAVAKRLAARGIKVRPGMVIGYLVLRGDGPISRRAIPAEEFDPSRHKYDAEYYIENQVLPAVLRILEA
FGYRKEDLRYQKTRQAGLDAWLKRKASL
Pyrococcus sp. ST04 (SEQ ID NO: 11)
MILDADYITEDGKPVIRLFKKENGEFKIEYDRTFKPYIYALLKDDSAIDEVRKVTAERHGKIVRIIDV
EKVKKKYLGRPIEVWKLYFEHPQDVPAIREKIREHPAVVEIFEYDIPFAKRYLIDKGIVPMDGDEELK
LLAFDIETLYHEGEEFGKGPILMISYADEEGAKVITWKRINLPYVEVVSSEREMIKRFLKVIREKDPD
VIITYNGDSFDFPYLVKRAEKLGIKLPLGRDGSPPKMQRLGDMNAVEIKGRIHFDLYHVVRRTINLPT
YTLEAVYEAIFGKPKEKVYAHEIAEAWETGKGLERVAKYSMEDAQVTYELGKEFFPMEVQLTRLVGQP
LWDVSRSSTGNLVEWYLLRKAYERNELAPNKPDEREYERRLRESYAGGYVKEPERGLWENIVYLDFRS
LYPSIIITHNVSPDTLNREGCRKYDIAPEVGHKFCKDVEGFIPSLLGHLLEERQKIKRKMKATINPVE
KKLLDYRQKAIKILANSYYGYYGYAKARWYCKECAESVTAWGREYIELVRKELEGKFGFKVLYIDTDG
LYATIPRGDPAEIKKKALEFVRYINEKLPGLLELEYEGFYRRGFFVTKKKYALIDEEDKIITRGLEIV
RRDWSEIAKETQAKVLEAILKEGNVEKAVKIVKEVTEKLMKYEVPPEKLVIYEQITRPLNEYKAIGPH
VAVAKRLAAKGVKVRPGMVIGYIVLRGDGPISKRAILAEEYDPRKNKYDAEYYIENQVLPAVLRILEA
FGYKKEDLKYQKSRQVGLGAWIKVKK
Pyrococcus sp. GB-D (SEQ ID NO: 12)
MILDADYITEDGKPIIRIFKKENGEFKVEYDRNFRPYIYALLKDDSQIDEVRKITAERHGKIVRIIDA
EKVRKKFLGRPIEVWRLYFEHPQDVPAIRDKIREHSAVIDIFEYDIPFAKRYLIDKGLIPMEGDEELK
LLAFDIETLYHEGEEFAKGPIIMISYADEEEAKVITWKKIDLPYVEVVSSEREMIKRFLKVIREKDPD
VIITYNGDSFDLPYLVKRAEKLGIKLPLGRDGSEPKMQRLGDMTAVEIKGRIHFDLYHVIRRTINLPT
YTLEAVYEAIFGKPKEKVYAHEIAEAWETGKGLERVAKYSMEDAKVTYELGREFFPMEAQLSRLVGQP
LWDVSRSSTGNLVEWYLLRKAYERNELAPNKPDEREYERRLRESYAGGYVKEPEKGLWEGLVSLDFRS
LYPSIIITHNVSPDTLNREGCREYDVAPEVGHKFCKDFPGFIPSLLKRLLDERQEIKRKMKASKDPIE
KKMLDYRQRAIKILANSYYGYYGYAKARWYCKECAESVTAWGREYIEFVRKELEEKFGFKVLYIDTDG
LYATIPGAKPEEIKKKALEFVDYINAKLPGLLELEYEGFYVRGFFVTKKKYALIDEEGKIITRGLEIV
RRDWSEIAKETQAKVLEAILKHGNVEEAVKIVKEVTEKLSKYEIPPEKLVIYEQITRPLHEYKAIGPH
VAVAKRLAARGVKVRPGMVIGYIVLRGDGPISKRAILAEEFDLRKHKYDAEYYIENQVLPAVLRILEA
FGYRKEDLRWQKTKQTGLTAWLNIKKK
SDS-0 (SEQ ID NO: 13)
MILDADYITEDGKPIIRIFKKENGEFKVEYDRNFRPYIYALLRDDSAIDEIKKITAQRHGKVVRIVET
EKIQRKELGRPIEVWKLYLEHPQDQPAIRDKIREHPAVVDIFEYDIPFAKRYLIDKGLTPAEGNEKLT
FLAVAIAALYHEGEEFGKGPVIMISYADEEGAKVITWKKIDLPYVEVVSSEREMIKRLIRVIKEKDPD
VIITYNGDNFDFPYLLKRAEKLGIKLLLGRDNSEPKMQKMGDSLAVEIKGRIHFDLFPVIRRTINLPT
YTLEAVYEAIFGKPKEKVYADEIAKAWETGEGLERVAKYSMEDAKVTYELGREFFPMEAQLARLVGQP
VWDVSRSSTGNLVEWFLLRKAYERNELAPNKPDEKEYERRLRESYEGGYVKEPEKGLWEGIVSLDFRS
AGPSIIITHNVSPDTLNREGSEEYDVAPKVGHRFSKDFPGFIPSLLGQLLEERQKIKKRMKESKDPVE
KKLLDYRQRVIKILANSYYGYYGYAKARWYSKESAESVSAWGRQYIDLVRRELEARGFKVLYIDTDGL
YATIPGVKDWEEVKRRALEFVDYINSKLPGVLELEYEGFYARGFFVTKKKYALIDEEGKIVTRGLEIV
RRDWSEIAKETQARVLEAILKHGNVEELVKIVKDVTEKLTNYEVPPEKLVIYEQITRPINEYKAIGPH
VAVAKRLMARGIKVKPGMVIGYIVLRGDGPISKRAISIEEFDPRKHKYDAEYYIENQVLPAVERILKA
FGYKREDLRWQKTKQVGLGAWIKVKKS

### EMBODIMENTS

Embodiment 1. A polymerase comprising an amino acid sequence that is at least 80% identical to a continuous 500 amino acid sequence within SEQ ID NO: 1; 45 comprising a first mutation at amino acid position 409 or an amino acid position corresponding to position 409; and at least one mutation at amino acid position 7 or an amino acid position corresponding to position 7, wherein the mutation at amino acid position 7 comprises histidine, lysine, or arginine; amino acid position 579 or an amino acid position corresponding to position 579, wherein the mutation at amino acid position 579 comprises leucine, isoleucine, valine, alanine, or glycine; amino acid position 588 or an amino acid position corresponding to position 588, wherein the mutation at amino acid position 588 comprises leucine, isoleucine, valine, alanine, or glycine; or amino acid position 742 or an amino acid position corresponding to position 742, wherein the mutation at amino acid position 742 comprises leucine, isoleucine, alanine, or glycine.

Embodiment 2. The polymerase of Embodiment 1, wherein the first mutation at amino acid position 409 or the amino acid position corresponding to position 409 is alanine, glutamine, tyrosine, phenylalanine, isoleucine, valine, cysteine, serine, or histidine.

Embodiment 3. The polymerase of Embodiments 1 or 2, comprising a glycine or alanine at amino acid position 410 or an amino acid position corresponding to position 410.

Embodiment 4. The polymerase of any one of Embodiments 1 to 3, comprising a proline, serine, alanine, glycine, valine, or isoleucine at amino acid position 411 or an amino acid position corresponding to position 411.

Embodiment 5. The polymerase of Embodiment 1, comprising: an alanine or serine at amino acid position 409 or the amino acid position corresponding to position 409; a glycine at amino acid position 410 or an amino acid position corresponding to position 410; and a proline, valine, glycine, isoleucine, or serine at amino acid position 411 or an amino acid position corresponding to position 411.

Embodiment 6. The polymerase of any one of Embodiments 1 to 5, wherein the mutation at amino acid position 588 or the amino acid corresponding to position 588 is leucine, isoleucine, valine, alanine, or glycine.

Embodiment 7. The polymerase of any one of Embodiments 1 to 5, wherein the mutation at amino acid position 588 or the amino acid position corresponding to position 588 is leucine, isoleucine, or valine.

Embodiment 8. The polymerase of any one of Embodiments 1 to 7, wherein the mutation at amino acid position 7 or the amino acid position corresponding to position 7 is histidine, lysine, or arginine.

Embodiment 9. The polymerase of any one of Embodiments 1 to 7, wherein the mutation at amino acid position 7 or the amino acid position corresponding to position 7 is histidine.

Embodiment 10. The polymerase of any one of Embodiments 1 to 9, wherein the mutation at amino acid position 579 or the amino acid position corresponding to position 579 is leucine, isoleucine, valine, alanine, or glycine.

Embodiment 11. The polymerase of any one of Embodiments 1 to 9, wherein the mutation at amino acid position 579 or the amino acid position corresponding to position 579 is glycine, arginine, histidine, or lysine.

Embodiment 12. The polymerase of any one of Embodiments 1 to 11, wherein the mutation at amino acid position 742 or the amino acid position corresponding to position 742 is leucine, isoleucine, alanine, or glycine.

Embodiment 13. The polymerase of any one of Embodiments 1 to 11, wherein the mutation at amino acid position 742 or the amino acid position corresponding to position 742 is alanine or glycine.

Embodiment 14. The polymerase of any one of Embodiments 1 to 13, further comprising a mutation at amino acid position 97 or an amino acid position corresponding to position 97; amino acid position 13 or an amino acid position corresponding to position 13; amino acid position 726 or an amino acid position corresponding to position 726; and/or amino acid position 241 or an amino acid position corresponding to position 241.

Embodiment 15. The polymerase of Embodiment 14, wherein the mutation at amino acid position 97 or the amino acid position corresponding to position 97 is cysteine, histidine, lysine, serine, threonine, or methionine.

Embodiment 16. The polymerase of Embodiment 14, wherein the mutation at amino acid position 97 or the amino acid position corresponding to position 97 is cysteine, histidine, or serine.

Embodiment 17. The polymerase of any one of Embodiments 14 to 16, wherein the mutation at amino acid position 13 or the amino acid position corresponding to position 13 is arginine or histidine.

Embodiment 18. The polymerase of any one of Embodiments 14 to 16, wherein the mutation at amino acid position 726 or the amino acid position corresponding to position 726 is aspartic acid, glutamic acid, asparagine, or glutamine.

Embodiment 19. The polymerase of any one of Embodiments 14 to 18, wherein the mutation at amino acid position 241 or the amino acid position corresponding to position 241 is leucine, isoleucine, alanine, valine, or glycine.

Embodiment 20. The polymerase of any one of Embodiments 1 to 19, further comprising a mutation at amino acid position 472 or the amino acid position corresponding to position 472, wherein the mutation is asparagine, aspartic acid, glutamic acid, or glutamine.

Embodiment 21. The polymerase of any one of Embodiments 1 to 19, further comprising a mutation at amino acid position 472 or the amino acid position corresponding to position 472, wherein the mutation is asparagine or glutamic acid.

Embodiment 22. The polymerase of any one of Embodiments 1 to 21, further comprising a mutation at amino acid position 469 or the amino acid position corresponding to position 469, wherein the mutation is threonine, serine, cysteine, or methionine.

Embodiment 23. The polymerase of any one of Embodiments 1 to 21, further comprising a mutation at amino acid position 469 or the amino acid position corresponding to position 469, wherein the mutation is asparagine, aspartic acid, glutamic acid, or glutamine.

Embodiment 24. The polymerase of any one of Embodiments 1 to 23, comprising a mutation at: amino acid position 520 or the amino acid position corresponding to position 520, wherein the mutation is histidine, lysine, or arginine; a mutation at amino acid position 465 or the amino acid position corresponding to position 465, wherein the mutation is asparagine, aspartic acid, glutamic acid, or glutamine; and/or a mutation at amino acid position 491 or the amino acid position corresponding to position 491, wherein the mutation is glycine, valine, leucine, or isoleucine. 25. The polymerase of any one of Embodiments 1 to 24, comprising a glutamine, valine, arginine, or alanine at amino acid position 93 or the amino acid position corresponding to position 93.

Embodiment 26. The polymerase of any one of Embodiments 1 to 25, comprising an alanine at amino acid position 141 or the amino acid position corresponding to position 141; and an alanine at amino acid position 143 or the amino acid position corresponding to position 143.

Embodiment 27. The polymerase of Embodiment 1, comprising: a leucine or isoleucine at amino acid position 588, or the amino acid position corresponding to position 588; a histidine at amino acid position 520, or the amino acid position corresponding to position 520; a glycine at amino acid position 580, or the amino acid position corresponding to position 580; a glutamic acid at amino acid position 465, or the amino acid position corresponding to position 465; a valine at amino acid position 491, or the amino acid position corresponding to position 491; or a glutamic acid at amino acid position 472, or the amino acid position corresponding to position 472.

Embodiment 28. The polymerase of Embodiment 1, comprising: a histidine at amino acid position 7, or the amino acid position corresponding to position 7; a valine at amino acid position 491, or the amino acid position corresponding to position 491; an arginine at amino acid position 13, or the amino acid position corresponding to position 13; a histidine at amino acid position 526, or the amino acid position corresponding to position 526; a valine at amino acid position 40, or the amino acid position corresponding to position 40; a glycine at amino acid position 579, or the amino acid position corresponding to position 579; a leucine at amino acid position 75, or the amino acid position corresponding to position 75; a leucine at amino acid position 588, or the amino acid position corresponding to position 588; a cysteine or a histidine at amino acid position 97, or the amino acid position corresponding to position 97; an isoleucine at amino acid position 606, or the amino acid position corresponding to position 606; an aspartic acid at amino acid position 149, or the amino acid position corresponding to position 149; an aspartic acid at amino acid position 635, or the amino acid position corresponding to position 635; an arginine at amino acid position 192, or the amino acid position corresponding to position 192; an aspartic acid at amino acid position 637, or the amino acid position corresponding to position 637; a glutamic acid at amino acid position 199, or the amino acid position corresponding to position 199; an aspartic acid at amino acid position 672, or the amino acid position corresponding to position 672; an isoleucine at amino acid position 241, or the amino acid position corresponding to position 241; an aspartic acid at amino acid position 726, or the amino acid position corresponding to position 726; a leucine at amino acid position 275, or the amino acid position corresponding to position 275; an alanine at amino acid position 742, or the amino acid position corresponding to position 742; a serine at amino acid position 316, or the amino acid position corresponding to position 316; a tyrosine at amino acid position 749, or the amino acid position corresponding to position 749; a glutamine at amino acid position 395, or the amino acid position corresponding to position 395; an aspartic acid at amino acid position 765, or the amino acid position corresponding to position 765; a threonine at amino acid position 469, or the amino acid position corresponding to position 469; an arginine at amino acid position 769, or the amino acid position corresponding to position 769; or an asparagine at amino acid position 472, or the amino acid position corresponding to position 472.

Embodiment 29. A method of incorporating a modified nucleotide into a nucleic acid sequence comprising combining in a reaction vessel: (i) a nucleic acid template, (ii) a nucleotide solution, and (iii) a polymerase, wherein the polymerase is a polymerase of any one of Embodiments 1 to 28.

Embodiment 30. A method of sequencing a nucleic acid sequence comprising: a. hybridizing a nucleic acid template with a primer to form a primer-template hybridization complex; b. contacting the primer-template hybridization complex with a DNA polymerase and modified nucleotides, wherein the DNA polymerase is the polymerase of any one of Embodiments 1 to 28, wherein the modified nucleotide comprises a detectable label; c. subjecting the primer-template hybridization complex to conditions which enable the polymerase to incorporate a modified nucleotide into the primer-template hybridization complex to form a modified primer-template hybridization complex; d. detecting the detectable label; thereby sequencing a nucleic acid sequence.

What is claimed is:

1. A polymerase comprising an amino acid sequence that is at least 80% identical to a continuous 500 amino acid sequence within SEQ ID NO: 1; comprising a first mutation at amino acid position 409 or an amino acid position corresponding to position 409; and at least one mutation at
amino acid position 7 or an amino acid position corresponding to position 7, wherein the mutation at amino acid position 7 comprises histidine, lysine, or arginine;
amino acid position 579 or an amino acid position corresponding to position 579, wherein the mutation at amino acid position 579 comprises leucine, isoleucine, valine, alanine, or glycine;
amino acid position 588 or an amino acid position corresponding to position 588, wherein the mutation at amino acid position 588 comprises leucine, isoleucine, valine, alanine, or glycine; or
amino acid position 742 or an amino acid position corresponding to position 742, wherein the mutation at amino acid position 742 comprises leucine, isoleucine, alanine, or glycine.

2. The polymerase of claim 1, wherein the first mutation at amino acid position 409 or the amino acid position corresponding to position 409 is alanine, glutamine, tyrosine, phenylalanine, isoleucine, valine, cysteine, serine, or histidine.

3. The polymerase of claim 1, comprising:
an alanine or serine at amino acid position 409 or the amino acid position corresponding to position 409;
a glycine at amino acid position 410 or an amino acid position corresponding to position 410; and
a proline, valine, glycine, isoleucine, or serine at amino acid position 411 or an amino acid position corresponding to position 411.

4. The polymerase of claim 1, wherein the mutation at amino acid position 588 or the amino acid corresponding to position 588 is leucine, isoleucine, valine, alanine, or glycine.

5. The polymerase of claim 1, wherein the mutation at amino acid position 7 or the amino acid position corresponding to position 7 is histidine, lysine, or arginine.

6. The polymerase of claim 1, wherein the mutation at amino acid position 579 or the amino acid position corresponding to position 579 is leucine, isoleucine, valine, alanine, or glycine.

7. The polymerase of claim 1, wherein the mutation at amino acid position 742 or the amino acid position corresponding to position 742 is leucine, isoleucine, alanine, or glycine.

8. The polymerase of claim 1, further comprising a mutation at amino acid position 97 or an amino acid position corresponding to position 97;
amino acid position 13 or an amino acid position corresponding to position 13;
amino acid position 726 or an amino acid position corresponding to position 726; and/or
amino acid position 241 or an amino acid position corresponding to position 241.

9. The polymerase of claim 14, wherein the mutation at amino acid position 97 or the amino acid position corresponding to position 97 is cysteine, histidine, lysine, serine, threonine, or methionine.

10. The polymerase of claim 14, wherein the mutation at amino acid position 13 or the amino acid position corresponding to position 13 is arginine or histidine.

11. The polymerase of claim 14, wherein the mutation at amino acid position 726 or the amino acid position corresponding to position 726 is aspartic acid, glutamic acid, asparagine, or glutamine.

12. The polymerase of claim 14, wherein the mutation at amino acid position 241 or the amino acid position corresponding to position 241 is leucine, isoleucine, alanine, valine, or glycine.

13. The polymerase of claim 1, further comprising a mutation at amino acid position 472 or the amino acid position corresponding to position 472, wherein the mutation is asparagine, aspartic acid, glutamic acid, or glutamine.

14. The polymerase of claim 1, further comprising a mutation at amino acid position 469 or the amino acid position corresponding to position 469, wherein the mutation is threonine, serine, cysteine, or methionine.

15. The polymerase of claim 1, comprising
a mutation at amino acid position 520 or the amino acid position corresponding to position 520, wherein the mutation is histidine, lysine, or arginine;
a mutation at amino acid position 465 or the amino acid position corresponding to position 465, wherein the mutation is asparagine, aspartic acid, glutamic acid, or glutamine; and/or
a mutation at amino acid position 491 or the amino acid position corresponding to position 491, wherein the mutation is glycine, valine, leucine, or isoleucine.

16. The polymerase of claim 1, comprising a glutamine, valine, arginine, or alanine at amino acid position 93 or the amino acid position corresponding to position 93.

17. The polymerase of claim 1, comprising:
a leucine or isoleucine at amino acid position 588, or the amino acid position corresponding to position 588;
a histidine at amino acid position 520, or the amino acid position corresponding to position 520;
a glycine at amino acid position 580, or the amino acid position corresponding to position 580;
a glutamic acid at amino acid position 465, or the amino acid position corresponding to position 465;
a valine at amino acid position 491, or the amino acid position corresponding to position 491; or
a glutamic acid at amino acid position 472, or the amino acid position corresponding to position 472.

18. The polymerase of claim 1, comprising:
a histidine at amino acid position 7, or the amino acid position corresponding to position 7;
a valine at amino acid position 491, or the amino acid position corresponding to position 491;
an arginine at amino acid position 13, or the amino acid position corresponding to position 13;
a histidine at amino acid position 526, or the amino acid position corresponding to position 526;
a valine at amino acid position 40, or the amino acid position corresponding to position 40;
a glycine at amino acid position 579, or the amino acid position corresponding to position 579;
a leucine at amino acid position 75, or the amino acid position corresponding to position 75;
a leucine at amino acid position 588, or the amino acid position corresponding to position 588,
a cysteine or a histidine at amino acid position 97, or the amino acid position corresponding to position 97;
an isoleucine at amino acid position 606, or the amino acid position corresponding to position 606,
an aspartic acid at amino acid position 149, or the amino acid position corresponding to position 149;
an aspartic acid at amino acid position 635, or the amino acid position corresponding to position 635;
an arginine at amino acid position 192, or the amino acid position corresponding to position 192;
an aspartic acid at amino acid position 637, or the amino acid position corresponding to position 637;
a glutamic acid at amino acid position 199, or the amino acid position corresponding to position 199;
an aspartic acid at amino acid position 672, or the amino acid position corresponding to position 672;
an isoleucine at amino acid position 241, or the amino acid position corresponding to position 241;
an aspartic acid at amino acid position 726, or the amino acid position corresponding to position 726;
a leucine at amino acid position 275, or the amino acid position corresponding to position 275;
an alanine at amino acid position 742, or the amino acid position corresponding to position 742;
a serine at amino acid position 316, or the amino acid position corresponding to position 316;
a tyrosine at amino acid position 749, or the amino acid position corresponding to position 749;
a glutamine at amino acid position 395, or the amino acid position corresponding to position 395;
an aspartic acid at amino acid position 765, or the amino acid position corresponding to position 765;
a threonine at amino acid position 469, or the amino acid position corresponding to position 469;
an arginine at amino acid position 769, or the amino acid position corresponding to position 769; or
an asparagine at amino acid position 472, or the amino acid position corresponding to position 472.

19. A method of incorporating a modified nucleotide into a nucleic acid sequence comprising combining in a reaction vessel: (i) a nucleic acid template, (ii) a nucleotide solution, and (iii) a polymerase, wherein the polymerase is a polymerase of any one of claim 1.

20. A method of sequencing a nucleic acid sequence comprising:
a. hybridizing a nucleic acid template with a primer to form a primer-template hybridization complex;
b. contacting the primer-template hybridization complex with a DNA polymerase and modified nucleotides, wherein the DNA polymerase is the polymerase of claim 1, wherein the modified nucleotide comprises a detectable label;
c. incorporating a modified nucleotide into the primer-template hybridization complex with the DNA polymerase to form a modified primer-template hybridization complex;
d. detecting the detectable label; thereby sequencing a nucleic acid sequence.
